(19) United States
(12) Patent Application Publication
(10) Pub. No.: US 2012/0058523 A1
(43) Pub. Date: Mar. 8, 2012

(54) TEMPERING OF CELLULOSIC BIOMASS

(75) Inventors: Kirk Pappan, Abilene, KS (US); Deisy Corridor, Manhattan, KS (US); Ramesh Nair, Manhattan, KS (US); Michael Blaylock, Purcellville, VA (US); Forrest Chumley, Manhattan, KS (US); David Lee, Washington, DC (US); Bruce Ferguson, Great Falls, VA (US)

(73) Assignee: EDENSPACE SYSTEMS CORPORATION, Manhattan, KS (US)

(21) Appl. No.: 13/201,846

(22) PCT Filed: Feb. 17, 2010
(86) PCT No.: PCT/US10/24505

§ 371 Date: Nov. 2, 2011

Related U.S. Application Data
(60) Provisional application No. 61/153,266, filed on Feb. 17, 2009.

(51) Int. Cl. C12P 19/14; C12P 19/02; C12P 19/00

(52) U.S. Cl. 435/99; 435/72; 435/105

(57) ABSTRACT

The present invention is directed to improved systems and methods for reducing costs and increasing yields of cellulosic ethanol. In particular, the present invention provides plants genetically transformed for increased biomass, expression of lignocellulolytic enzyme polypeptides, and/or simplification of harvesting and downstream processing. Also provided are methods for processing biomass from these transgenic plants that involve less severe and/or less expensive pre-treatment protocols that are typically employed. Such methods allow, among other things, reduced costs associated with externally applied lignocellulolytic enzyme polypeptides.

### RELATED APPLICATION INFORMATION

The present application claims benefit of, and priority to, U.S. provisional application Ser. No. 61/153,266, filed on Feb. 17, 2009, the contents of which are herein incorporated by reference in their entirety.

### GOVERNMENT SUPPORT

This invention was made with U.S. government support under the United States Department of Agriculture and Department of Energy Biomass Grant No. DE-PS36-06GO96002F. The government of the United States of America has certain rights in the invention.

### SEQUENCE LISTING

The present specification makes reference to a Sequence Listing (submitted electronically as a .txt file named “SequenceListing.txt” concurrently with other documents associated with this application on Feb. 16, 2010). The .txt file was generated on Feb. 16, 2010 and is 39 kb in size. The entire contents of the Sequence Listing are herein incorporated by reference.

### BACKGROUND

Rising oil prices have increased the cost-competitiveness of ethanol as a fuel, which has captured a substantial share of the U.S. fuel market. Federal agencies such as the U.S. Department of Agriculture (USDA) have begun to implement programs of preferred procurement of biofuels such as ethanol as a fuel additive. The potential market for the biofuels in industries such as transportation fuel is one of the largest in the U.S. economy.

The ability to produce ethanol from low-cost biomass has been called the “key” to making ethanol competitive as a gasoline additive (J. DiPardo, “Outlook for biomass ethanol production and demand”, EIA Forecasts, 2002). Joint USDA/DOE studies forecast the ability to produce up to approximately 100 billion gallons per year from forestland and agricultural land combined (R. D. Perlack et al., “Biomass as feedstock for a bioenergy and bioproducts industry: The technical feasibility of a billion-ton annual supply”, 2005, ORNL/TM-2005/66). Lignocellulosic production would also help to increase the net energy balance of corn ethanol. The conversion of lignocellulosic feedstocks into ethanol has advantages including ready availability of large amounts of feedstock, avoidance of burning or land filling the materials, and relatively easy conversion of glucose (produced by hydrolysis of cellulose) into ethanol. Most studies show substantial energy advantages of using cellulosic feedstocks (e.g., Farrell et al., Science, 2006, 311: 506-508).

Today most fuel ethanol is produced from corn (maize) grain, which is milled, treated with amylase enzymes to hydrolyze starch to sugars, fermented, and distilled. While substantial progress has been made in reducing costs of ethanol production, substantial challenges remain. One such challenge is to reduce the costs of pretreating the biomass to remove lignin and hemicellulose. Another challenge is to reduce the cost of microbially-produced enzymes that are often used in processing lignocellulosic biomass.

Improved techniques are still needed to reduce the cost of biofuel feedstocks for ethanol production.

### SUMMARY

Standard industrial processing of lignocellulosic biomass for fuel production typically involves a pretreatment phase (during which lignin and hemicellullose is removed to promote accessibility of cellulose to enzyme polypeptides for hydrolysis) and a treatment phase (during which cellulose is hydrolyzed by cellulases into fermentable sugar monomers such as glucose). The inventors had previously developed systems for processing lignocellulosic biomass using transgenic plants expressing lignocellulolytic enzyme polypeptides. Using such systems, it was possible to reduce the severity of pretreatment conditions and/or amount of external cellulase enzyme added during the treatment phase in order to achieve a given level of hydrolysis. Such improvements translate to reduced overall costs.

The present invention encompasses the recognition that even mild pretreatment conditions may cause denaturation of lignocellulolytic enzyme polypeptides, thus limiting the efficacy of the pretreatment and treatment phases. The inventors postulated that yield and cost reduction could be further enhanced if the biomass were conditioned for pretreatment and treatment during an additional processing phase prior to pretreatment. The additional processing phase, which the inventors have called ‘tempering,’ may achieve one or more results such as activation of endoplant enzyme polypeptides, increased susceptibility of lignin and hemicellulose to traditional pretreatment, facilitating reduced severity of pretreatment to achieve acceptable glucan conversion yields, improved hydrolysis and conversion after pretreatment, and increased accessibility of polysaccharides (e.g., cellulose).

The inventors have developed methods for cost-effective processing of lignocellulosic biomass comprising steps of: tempering a sample of plant biomass under conditions to promote activation of lignocellulolytic enzyme polypeptides present in the sample of plant biomass; pretreating the sample under conditions to promote accessibility of celluloses within the lignocellulosic biomass; and treating the pretreated sample under conditions that promote hydrolysis of cellulose to fermentable sugars. In such methods, the sample of plant biomass is obtained from at least one trangenic plant, the genome of which is augmented with a recombinant polynucleotide encoding at least one lignocellulolytic enzyme polypeptide operably linked to a promoter sequence, wherein the polynucleotide is optimized for expression in the plant.

In some embodiments, tempering comprises a process selected from the group consisting of ensilement, grinding, pelleting, microwaving, sonication, incubation at a particular temperature or at particular temperatures, incubation at a particular pH, and combinations thereof. The sample of biomass may be, for example, in solid form and/or in a liquid slurry during the tempering step.

These and other objects, advantages and features of the present invention will become apparent to those of ordinary skill in the art having read the following detailed description.

### BRIEF DESCRIPTION OF THE DRAWING

FIG. 1 is a map of the pBI121 vector used in the transformation of tobacco as reported in Example 1. The following sequences are abbreviated; NOS promoter (N-p), neomycin phospho-transferase II (NPTII), NOS terminator (N-t), cauliflower mosaic virus 35S promoter (35S), β-glucuronidase (GUS), agrobacteria right border sequence (RB), left border (LB).

FIG. 2 is a map of the pBI121-E1 vector in between the right and left border sequences used in the transformation of tobacco as reported in Example 1. The E1 construct contains the VSPβ signal peptide fused to the N-terminus of the E1 catalytic domain. The following sequences are abbreviated; NOS promoter (N-p), neomycin phospho-transferase II (NPTII), NOS terminator (N-t), cauliflower mosaic virus 35S promoter (35S), β-glucuronidase (GUS), agrobacteria right border sequence (RB), left border (LB).

FIG. 3 is a graph showing glucose production from dried E1-FLC and W38 (wild-type) tobacco biomass equilibrated in citrate buffer (pH 4.5) for 24 hours at 50° C. with or without added Cellulase AN, glucoamylase, and hemicellulase. Error bars indicate ±1 standard deviation (n=3).

FIG. 4 shows the results of Southern blot analysis of genomic DNA from corn plants, probed with the E1-cat. Lane 1:10 pg of Sac 1 E1 fragment from pMZ766; Lanes 2-3: untransformed corn control (lane 2: DNA undigested and lane 3: DNA digested); Lanes 4-13: five independent pMZ766 transformants; (lanes 4, 6, 8, 10, and 12: DNA not digested; lanes 5, 7, 9, 11, and 13: DNA digested with Sac I). Size of bands is 1 kb.

FIG. 5 shows Western blots of transgenic corn as compared to transgenic rice and transgenic tobacco. Upper panel: 1 μg total soluble protein from transgenic maize plants expressing E1 was assayed in each lane. Lanes: +, positive tobacco control (Austin-Phillips); −C, negative maize control (untransformed); 1-10, transgenic maize lines representing at least 5 different transformation eventws; 11, transgenic rice. 1-5: transformed with pMZ766E1cat and pBY520; 6: transformed with pMZ766E1cat and pDM302; 7: transformed with E1 binary vector construct 2 and pBY520; 8-9: transformed with pMZ766E1cat and pBY520; 10: transformed with pMZ766E1cat and pDME302. 1, 2, 5, and 6 are expressing E1. The others do not have detectable E1. Lower panel: 2 μg total soluble protein from transgenic maize plants expressing E1 was assayed in each lane. Lanes: +, positive tobacco control (Austin-Phillips); −C negative maize control (untransformed); 1-6, transgenic maize lines representing at least 4 different transformation events; 7, transgenic rice. 1-3: transformed with pMZ766E1cat and BY520; 4: transformed with pMZ766E1cat and pDM302; 5: transformed with E1 binary vector construct 2 and pBY520; 6: transformed with pMZ766E1cat and pDM302. In this blot, only 3 is expressing a faint amount of E1. The others do not have detectable E1.

FIG. 6(A) is a picture of E1 transgenic maize leaf tissue obtained by immunofluorescent confocal laser microscopy image microscopy using the E1 primary antibody and the FITC anti-mouse secondary antibody. This picture shows that E1 transgenic leaf tissue exhibits apparent storage of E1 in the plant apoplast. FIG. 6(B) is a confocal microscopy image of leaf tissue from an untransformed control maize leaf showing no expression of E1 enzyme.

FIG. 7 shows the cellulase activity of crude extract from transgenic tobacco expressing E1. The red carboxy-methyl-cellulose in the Petri dish has been hydrolyzed by the application of E1 extract or commercially available cellulase enzymes (Cellulase AN, BIO-CAT) to form clear areas. No cellulase activity was observed from wild type tobacco extract.

FIG. 8 shows a graph illustrating the increased production of glucose from hydrolysis of transgenic tobacco biomass expressing the E1 endoglucanase compared to wild-type biomass, especially following pretreatment. Glucose levels were measured from E1, wild-type (W38), and Avicel samples (see Example 4) that were digested with ACCELLERASE™ 1000 enzyme cocktail either after pretreatment (E1-1 and W38) or without pretreatment (E1-Orig and W38 Orig). Avicel is a commercially available cellulose substrate that was used as a control to measure effectiveness of pretreatment and hydrolysis reactions.

FIG. 9 shows two pictures allowing visualization of E1 hydrolysis of CMC (carboxyl-methyl-cellulose) using Congo Red staining Leaf tips of untransformed (left) and transgenic E1 (right) tobacco (A) before and (B) after incubation at 65° C. for thirty minutes and the staining with Congo Red.

FIG. 10 presents data showing glucan conversion rates of E1 transgenic and wildtype corn stover. Transgenic E1 biomass is more readily hydrolyzable to glucose than wild type biomass is. E1 corn under low (15 mg enzyme/g biomass) and high (100 mg/g) external enzyme loading conditions consistently provided higher levels of glucan conversion than untransformed (WT) corn across a wide range of pretreatment temperatures. Samples were pretreated for 10 minutes, neutralized, and hydrolyzed for 24 hours with external enzymes.

FIG. 11 shows results from tempering experiments. Samples from wild type (WT), E1 (a glucanase) transgenic, Xyn Z (a xylanase) transgenic, and E1 and Xyn Z double transgenic tobacco were tempered by incubation at 85° C. before digestion with commercial enzyme cocktail (Novozymes Celluclast 1.5 L). Digestibility was assayed using a modified in vitro dry matter digestibility (IVDMD) assay. Tempering slurried tobacco before enzyme hydrolysis improved the digestibility of transgenic tobacco. Column labels: a, p<0.05 non-tempered vs. tempered in the same sample; b, not significant non-tempered vs. tempered in the same sample; c, p<0.05 versus WT 2 tempered sample; d, not significant versus WT 2 tempered sample.

FIG. 12 shows results from tempering experiments on double transgenic (E1 and Xyn Z) tobacco at a variety of timepoints. As in FIG. 11, biomass was incubated at 85° C. before digestion with commercial enzyme cocktail. Even with short tempering periods of 5 hours, tempering selectively enhanced digestibility of double transgenic biomass by Novozymes Celluclast 1.5 L.

FIG. 13 shows high performance liquid chromatography (HPLC) profiles of supernatants collected from tobacco biomass slurries after tempering (by incubation at 85° C. for 15 hours). Most sugars released during tempering were water soluble oligosaccharides (left panel). Hydrolysis of supernatant sugars with H₂SO₄ (E1-H and Xyl-E1-H; right panel) increases glucose, arabinose, and mannose monomer concentration.

FIG. 14 depicts sugar yields of ensiled tobacco after enzyme hydrolysis without pre-treatment. Data is shown for various timepoints of enzyme hydrolysis. Ensilement of E1 tobacco for 20 days at 37° C. increased efficiency of enzyme hydrolysis.

FIG. 15 depicts sugar yield of ensiled tobacco after pretreatment and enzyme hydrolysis. Pretreatment was performed at 121° C. for 60 minutes in 0.5% acid. Ensilement increased sugar yield of E1 tobacco compared to that of unensiled E1 tobacco.

FIG. 16 is a process flow diagram of a method to handle biomass through harvest of biomass to distillation of ethanol and other byproducts. Both field operations and plant operations are indicated in the diagram and the integrated process is designed to use endoplant enzymes such as Edenspace's endoplant enzymes to (1) simplify postharvest handling of biomass, (2) reduce severity and cost of biomass pretreatment and (3) increase efficiency and reduce cost of enzymatic hydrolysis and ethanolic fermentation.

FIG. 17 expands on the pretreatment step in FIG. 16 to illustrate how pentose and hexose sugars will be separated after pretreatment and processed independently to maximize yields of fermentable sugars.

FIG. 18 depicts glucan yield measurements at various pretreatment temperatures from E1 endoglucanase transgenic corn stover hydrolyzed with low (0.2 mL/g glucan) and high (0.5 mL/g glucan) doses of ACCELLERASE™ 1000 enzyme.

FIG. 19 depicts glucan yield measurements from CBHE cellobiohydrolase I transgenic corn stover hydrolyzed with low (0.2 mL/g glucan) and high (0.5 mL/g glucan) doses of ACCELLERASE™ 1000 enzyme.

FIG. 20 depicts glucan release measurements from CBHE transgenic corn stover that was tempered by acceleration.

FIG. 21 depicts E1 endoglucanase activity measurements in corn stover and grain after 90 days of ensilement in the upper panel and results from a Western blot using anti-E1 primary antibody in the lower panel.

FIG. 22 depicts measurements of reducing sugar released after enzymatic hydrolysis of WT and E1 endoglucanase transgenic tobacco biomass that had been ensiled as compared to unensiled biomass.

FIG. 23 depicts measurements of reducing sugar released after dilute acid pretreatment and enzymatic hydrolysis of WT and E1 endoglucanase transgenic tobacco biomass that had been ensiled as compared to unensiled biomass.

FIG. 24 depicts glucan yield measurements from E1 endoglucanase transgenic switchgrass that was tempered by ensilement and by acceleration, then hydrolyzed with ACCELLERASE™1000.

FIG. 25 depicts ethanol production measurements from E1 endoglucanase transgenic corn stover and switchgrass in gallons of ethanol per dry ton (left panel) and in gallons of ethanol per acre (right panel).

FIG. 26 is a diagram showing an integrated process that could be used to increase glucan conversion, increase ethanol yield, reduce inhibitory compound formation, decrease severity of pretreatment, and lower the loading of commercial enzymes during saccharification.

FIG. 27 depicts results from an assay for E1 endoglucanase activity in corn biomass that had been tempered in alkaline conditions.

FIG. 28 depicts glucose release measurements and digestibility calculations from XylE (an endoxylanase from Acidothermus cellulolyticus) transgenic corn stover that had been tempered in alkaline conditions with or without peroxide.

FIG. 29 depicts results from an assay of E1 endoglucanase activity in switchgrass that had been subject to (1) no acceleration or pretreatement; (2) acceleration at pH 5.0; or (3) dilted acid pretreatment (top panel). The lower panel presents results from Western blots using anti-E1 primary antibodies.

FIG. 30 depicts, in the upper panel, liquid recovery (as a percentage of starting volume) after extraction of E1 enzyme from E1 transgenic switchgrass biomass samples that were centrifuged or pressed following acceleration. The lower panel depicts results from a Western blot using anti-E1 primary antibody.

FIG. 31 depicts results from an assay of E1 endoglucanase activity in samples obtained by extraction from E1 endoglucanase transgenic poplar and concentration by a variety of methods.

FIG. 32 depicts digestibility calculations from CBHE transgenic poplar biomass that had been subject to tempering (i.e., acceleration in this case), pretreatment, both, or neither.

FIG. 33 depicts glucan conversion measurements (indicating degree of enzyme hydrolysis by ACCELLERASE™ 1000) of tobacco biomass after tempering at 80° C. for 15 hours. Samples include biomass from transgenic tobacco expressing E1 endoglucanase, xylanase (Xyn), or both. Some samples were pretreated (PT).

FIG. 34 depicts digestibility calculations from E1+XynZ double transgenic biomass that had been subject to tempering (i.e., accleration in this case), pretreatment, both, or neither.

FIG. 35 depicts E1 endoglucanase activity as measured by a 4-methylumbelliferyl cellobioside assay (upper panel) and E1 endoglucanase expression as assessed by Western blot (lower panel) in corn stover and cobs from E1 endoglucanase transgenic corn plants.

FIG. 36 depicts E1 endoglucanase activity as measured by a 4-methylumbelliferyl cellobioside assay (upper panel) and E1 endoglucanase expression as assessed by Western blot (lower panel) in milled grain (corn seed) from E1 endoglucanase transgenic corn.

FIG. 37 depicts E1 endoglucanase activity as measured by a 4-methylumbelliferyl cellobioside assay (upper panel) and E1 endoglucanase expression as assessed by Western blot (lower panel) in leaf, stem, grain, and hull from E1 endoglucanase transgenic Sorghum.

FIG. 38 depicts digestibility calculations from E1 endoglucanase transgenic corn stover and grain pericarp that had been subject to acceleration.

### DEFINITIONS

Throughout the specification, several terms are employed that are defined in the following paragraphs.

As used herein, the terms “about” and “approximately,” in reference to a number, is used herein to include numbers that fall within a range of 20%, 10%, 5%, or 1% in either direction (greater than or less than) the number unless otherwise stated or otherwise evident from the context (except where such number would exceed 100% of a possible value).

As used herein, the term “acceleration,” when used in reference to a tempering process, refers to a process of activating thermostable enzymes in biomass, e.g., transgenic biomass. In certain embodiments, acceleration comprises hydrating biomass (if it is not already hydrated) and incubating the biomass with heat to activate such thermostable enzymes. In some embodiments, the biomass is incubated at a temperature greater than 65° C., such as about 70° C., about 75° C., about 80° C., or greater than 80° C.

As used herein, the term “ensilement” refers to an anaerobic fermentation process used to preserve forages, immature grain crops, and other biomass crops for feed and biofuels. In some embodiments, the crop is chopped and packed while at about 60-80% moisture and put into containers (such as, for example, silos) to exclude air. In some such embodiments (for example, for biofuel production), the crop is chopped, moisture content is increased by adding water as needed, and the crop is packed for storage and/or shipping in a manner to exclude air. In some embodiments, ensilement comprises incubation at an increased temperature to activate thermophilic enzymes and promote autohydrolysis. In some embodiments, yeast and/or other preservatives is/are added during ensilement of biofuel crops to minimize loss of sugars and accumulation of lactic acid.

As used herein, the phrase “externally applied,” when used to describe enzyme polypeptides used in the processing of biomass, refers to enzyme polypeptides that are not produced by the organism whose biomass is being processed. “Externally applied” enzyme polypeptides as used herein does not include enzyme polypeptides that are expressed (whether endogenously or transgenically) by the organism (e.g., plant) from which the biomass is obtained.

As used herein, the term “extract,” when used as noun, refers to a preparation from a biological material (such as lignocellulosic biomass) in which a substantial portion of proteins are in solution. In some embodiments of the invention, the extract is a crude extract, e.g., an extract that is prepared by disrupting cells such that proteins are solubilized and optionally removing debris, but not performing further purification steps. In some embodiments of the invention, the extract is further purified in that certain substances, molecules, or combinations thereof are removed.

As used herein, the term “gene” refers to a discrete nucleic acid sequence responsible for a discrete cellular product and/or performing one or more intracellular or extracellular functions. More specifically, the term “gene” refers to a nucleic acid that includes a portion encoding a protein and optionally encompasses regulatory sequences, such as promoters, enhancers, terminators, and the like, which are involved in the regulation of expression of the protein encoded by the gene of interest. The gene and regulatory sequences may be derived from the same natural source, or may be heterologous to one another. The definition can also include nucleic acids that do not encode proteins but rather provide templates for transcription of functional RNA molecules such as tRNAs, rRNAs, etc. Alternatively, a gene may define a genomic location for a particular event/function, such as the binding of proteins and/or nucleic acids.

As used herein, the term “gene expression” refers to the conversion of the information, contained in a gene, into a gene product. A gene product can be the direct transcriptional product of a gene (e.g., mRNA, tRNA, rRNA, antisense RNA, ribozyme structural RNA or any other type of RNA) or a protein produced by translation of an mRNA. Gene products also include RNAs that are modified by processes such as capping, polyadenylation, methylation, and editing, proteins post-translationally modified, and proteins modified by, for example, methylation, acetylation, phosphorylation, ubiquitination, ADP-ribosylation, myristilation, and glycosylation.

The terms “genetically modified” and “transgenic” are used herein interchangeably. A transgenic or genetically modified organism is one that has a genetic background which is at least partially due to manipulation by the hand of man through the use of genetic engineering. For example, the term “transgenic cell”, as used herein, refers to a cell whose DNA contains an exogenous nucleic acid not originally present in the non-transgenic cell. A transgenic cell may be derived or regenerated from a transformed cell or derived from a transgenic cell. Exemplary transgenic cells in the context of the present invention include plant calli derived from a stably transformed plant cell and particular cells (such as leaf, root, stem, or reproductive cells) obtained from a transgenic plant. A “transgenic plant” is any plant in which one or more of the cells of the plant contain heterologous nucleic acid sequences introduced by way of human intervention. Transgenic plants typically express DNA sequences, which confer the plants with characters different from that of native, non-transgenic plants of the same strain. The progeny from such a plant or from crosses involving such a plant in the form of plants, seeds, tissue cultures and isolated tissue and cells, which carry at least part of the modification originally introduced by genetic engineering, are comprised by the definition.

As used herein, the term “hydrolysis” refers to a reaction performed by one or more cellulase enzymes to break down cellulose in plant biomass to fermentable sugar monomers such as glucose. In some embodiments, hydrolysis is performed by a suite of enzymes; in some such embodiments, the suite of enzymes comprise enzymes from more the one class of cellulase.

As used herein, the term “lignocellulolytic enzyme polypeptide” refers to a polypeptide that disrupts or degrades lignocellulose, which comprises cellulose, hemicellulose, and lignin. The term “lignocelluloytic enzyme polypeptide” encompasses, but is not limited to cellobiohydrolases, endoglucanases, β-D-glucosidases, xylanases, arabinofuranosidases, acetyl xylan esterases, glucuronidases, mannanases, galactanases, arabinases, lignin peroxidases, manganese-dependent peroxidases, hybrid peroxidases, laccases, ferulic acid esterases and related polypeptides. In some embodiments, disruption or degradation of lignocellulose by a lignocellulolytic enzyme polypeptide leads to the formation of substances including monosaccharides, disaccharides, polysaccharides, and phenols. In some embodiments, a lignocellulolytic enzyme polypeptide shares at least 50%, 60%, 70%, 80% or more overall identity with a polypeptide whose amino acid sequence as set forth in Table 1 (see page 16). Alternatively or additionally, in some embodiments, a lignocellulolytic enzyme polypeptide shows at least 90%, 95%, 96%, 97%, 98%, 99%, or greater identity with at least one sequence element found in a polypeptide whose amino acid sequence is set forth in Table 1, which sequence element is at least 4, 5, 6, 7, 8, 9, 10, 11, 12, 13, 14, 15, 16, 17, 18, 19, 20 or more amino acids long. It will be appreciated that the present invention describes use of lignocellulolytic enzyme polypeptides generally, but also of particular lignocellulolytic enzyme polypeptides (e.g., Acidothermus cellulolyticus E1 endo-1,4-β-glucanase polypeptide, Acidothermus cellulolyticus xylE polypeptide, Acidothermus cellulolyticus

gux 1 polypeptide, Acidothermus cellulolyticus avilIl polypeptide, Talaromyces emersonii cbhE polypeptide, and Pyrococcus furiosus faeE (ferulic acid esterase) polypeptide).

As used herein, the term “nucleic acid construct” refers to a polynucleotide or oligonucleotide comprising nucleic acid sequences not normally associated in nature. A nucleic acid construct of the present invention is prepared, isolated, or manipulated by the hand of man. The terms “nucleic acid”, “polynucleotide” and “oligonucleotide” are used herein interchangeably and refer to a deoxyribonucleotide (DNA) or ribonucleotide (RNA) polymer either in single- or double- stranded form. For the purposes of the present invention, these terms are not to be construed as limited with respect to the length of the polymer and should also be understood to encompass analogs of DNA or RNA polymers made from analogs of natural nucleotides and/or from nucleotides that are modified in the base, sugar and/or phosphate moieties.

As used herein, the term “operably linked” refers to a relationship between two nucleic acid sequences wherein the expression of one of the nucleic acid sequences is controlled by, regulated by or modulated by the other nucleic acid sequence. Preferably, a nucleic acid sequence that is operably linked to a second nucleic acid sequence is covalently linked, either directly or indirectly, to such second sequence, although any effective three-dimensional association is acceptable. A single nucleic acid sequence can be operably linked to multiple other sequences. For example, a single promoter can direct transcription of multiple RNA species.

As will be clear from the context, the term “plant”, as used herein, can refer to a whole plant, plant parts (e.g., cuttings, tubers, pollen), plant organs (e.g., leaves, stems, flowers, roots, fruits, branches, etc.), individual plant cells, groups of plant cells (e.g., cultured plant cells), protoplasts, plant extracts, seeds, and progeny thereof. The class of plants which can be used in the methods of the present invention is as broad as the class of higher plants amenable to transformation techniques, including both monocotyledonous and dicotyledonous plants, as well as certain lower plants such as algae. The term includes plants of a variety of a ploidy levels, including polyploid, diploid and haploid. In certain embodiments of the invention, plants are green field plants. In other embodiments, plants are grown specifically for “biomass energy”. For example, suitable plants include, but are not limited to, corn, switchgrass, sorghum, miscanthus, sugarcane, poplar, pine, wheat, rice, soy, cotton, barley, turf grass, tobacco, bamboo, rape, sugar beet, sunflower, willow, and eucalyptus. Using transformation methods, genetically modified plants, plant cells, plant tissue, seeds, and the like can be obtained.

The term “polypeptide”, as used herein, generally has its art-recognized meaning of a polymer of at least three amino acids. However, the term is also used to refer to specific functional classes of polypeptides, such as, for example, lignocellulolytic enzyme polypeptides (including, for example, Acidothermus cellulolyticus E1 endo-1,4-β-glucanase polypeptide, Acidothermus cellulolyticus xylE polypeptide, Acidothermus cellulolyticus gux1 polypeptide, Acidothermus cellulolyticus aviIII polypeptide, Talaromyces emersonii cbhE polypeptide, and Pyrococcus furiosus faeE (ferulic acid esterase) polypeptide). For each such class, the present specification provides specific examples of known sequences of such polypeptides. Those of ordinary skill in the art will appreciate, however, that the term “polypeptide” is intended to be sufficiently general as to encompass not only polypeptides having the complete sequence recited herein (or in a reference or database specifically mentioned herein), but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Other regions of similarity and/or identity can be determined by those of ordinary skill in the art by analysis of the sequences of various polypeptides presented herein.

As used herein, the term “pretreatment” refers to a thermo-chemical process to remove lignin and hemicellulose bound to cellulose in plant biomass, thereby increasing accessibility of the cellulose to cellulases for hydrolysis. Common methods of pretreatment involve using dilute acid (such as, for example, sulfuric acid), ammonia fiber expansion (AFEX), steam explosion, lime, and combinations thereof.

As used herein, the terms “promoter” and “promoter element” refer to a polynucleotide that regulates expression of a selected polynucleotide sequence operably linked to the promoter, and which effects expression of the selected polynucleotide sequence in cells. The term “plant promoter”, as used herein, refers to a promoter that functions in a plant. In some embodiments of the invention, the promoter is a constitutive promoter, i.e., an unregulated promoter that allows continual expression of a gene associated with it. A constitutive promoter may in some embodiments allow expression of an associated gene throughout the life of the plant. Examples of constitutive plant promoters include, but are not limited to, rice actl promoter, Cauliflower mosaic virus (CaMV) 35S promoter, and nopaline synthase promoter from Agrobacterium tumefaciens. In some embodiments of the invention, the promoter is a tissue-specific promoter that selectively functions in a part of a plant body, such as a flower. In some embodiments of the invention, the promoter is a developmentally specific promoter. In some embodiments of the invention, the promoter is an inducible promoter. In some embodiments of the invention, the promoter is a senescence promoter, i.e., a promoter that allows transcription to be initiated upon a certain event relating to the age of the organism.

As used herein, the term “protoplast” refers to an isolated plant cell without cell walls which has the potency for regeneration into cell culture or a whole plant.

As used herein, the term “regeneration” refers to the process of growing a plant from a plant cell (e.g., plant protoplast, plant callus or plant explant).

As used herein, the term “stably transformed”, when applied to a plant cell, callus or protoplast refers to a cell, callus or protoplast in which an inserted exogenous nucleic acid molecule is capable of replication either as an autonomously replicating plasmid or as part of the host chromosome. The stability is demonstrated by the ability of the transformed cells to establish cell lines or clones comprised of a population of daughter cells containing the exogenous nucleic acid molecule.

As used herein, the term “tempering” refers to a process to condition lignocellulosic biomass prior to pretreatment so as to favor improved yield from hydrolysis and/or allow use of less severe pretreatment conditions without sacrificing yield. In some embodiments, the lignocellulosic biomass transgenically expresses a lignocellulolytic enzyme polypeptide and tempering facilitates activation of the lignocellulolytic enzyme polypeptide. In some embodiments, tempering facilitates improved yield from subsequent hydrolysis as compared to yield obtained from processing without tempering. In some embodiments, tempering facilitates comparable or improved yield from subsequent hydrolysis using less severe pretreatment conditions than would be required without tempering. In some embodiments, tempering comprises a process selected from the group consisting of ensilement, grinding, pelleting, forming a warm water suspension and/or slurry, incubating at a specific temperature, incubating at a specific pH, and combinations thereof. In some embodiments, tempering comprises separating liquid from a slurry that contains soluble sugars and crude enzyme extracts and re-addition of the separated liquid back to the solid biomass after pretreatment. Specific conditions for tempering may depend on specific traits (such as, e.g., traits of the transgene) of the biomass.

As used herein, the term “transformation” refers to a process by which an exogenous nucleic acid molecule (e.g., a vector or recombinant DNA molecule) is introduced into a recipient cell, callus or protoplast. The exogenous nucleic acid molecule may or may not be integrated into (i.e., covalently linked to) chromosomal DNA making up the genome of the host cell, callus or protoplast. For example, the exogenous polynucleotide may be maintained on an episomal element, such as a plasmid. Alternatively, the exogenous polynucleotide may become integrated into a chromosome so that it is inherited by daughter cells through chromosome replication. Methods for transformation include, but are not limited to, electroporation, magnetoporation, Ca²⁺ treatment, injection, particle bombardment, retroviral infection, and lipofection.

The term “transgene”, as used herein, refers to an exogenous gene which, when introduced into a host cell through the hand of man, for example, using a process such as transformation, electroporation, particle bombardment, and the like, is expressed by the host cell and integrated into the cell's DNA such that the trait or traits produced by the expression of the transgene is inherited by the progeny of the transformed cell. A transgene may be partly or entirely heterologous (i.e., foreign to the cell into which it is introduced). Alternatively, a transgene may be homologous to an endogenous gene of the cell into which it is introduced, but is designed to be inserted (or is inserted) into the cell's genome in such a way as to alter the genome of the cell (e.g., it is inserted at a location which differs from that of the natural gene or its insertion results in a knockout). A transgene can also be present in a cell in the form of an episome. A transgene can include one or more transcriptional regulatory sequences and other nucleic acids, such as introns. Alternatively or additionally, a transgene is one that is not naturally associated with the vector sequences with which it is associated according to the present invention.

### DETAILED DESCRIPTION OF CERTAIN EMBODIMENTS

As mentioned above, the present invention relates to improved systems and strategies for reducing costs and increasing yields of ethanol production from lignocellulosic biomass.

### I. Lignocellulolytic Enzyme Polypeptides

In one aspect, the present invention provides plants engineered (i.e., genetically transformed) to produce one or more lignocellulolytic enzyme polypeptides. Suitable lignocellulolytic enzyme polypeptides include enzymes that are involved in the disruption and/or degradation of lignocellulose. Lignocellulosic biomass is a complex substrate in which crystalline cellulose is embedded within a matrix of hemicellulose and lignin. Lignocellulose represents approximately 90% of the dry weight of most plant material with cellulose making up between 30% to 50% of the dry weight of lignocellulose and hemicellulose making up between 20% and 50% of the dry weight of lignocellulose.

Disruption and degradation (e.g., hydrolysis) of lignocellulose by lignocellulolytic enzyme polypeptides leads to the formation of substances including monosaccharides, disaccharides, polysaccharides and phenols. Lignocellulolytic enzyme polypeptides include, but are not limited to, cellulases, hemicellulases and ligninases. Representative examples of lignocellulolytic enzyme polypeptides are presented in Table 1.

TABLE 1
Examples of lignocellulolytic enzyme polypeptides that
may be used in accordance with the invention

| | | | GenBank |
|---|---|---|---|
| | | | Accession |
| | | | Number/ |
| Gene | Microbial | Amino Acid Sequence of Exemplary | SEQ ID |
| name | species | Lignocellulolytic Enzyme Polypeptide | NO. |
| E1 | Acidothermus | AGGGYWHTSGREILDANNVPVRIAGINWFGFETCNYVVHGLWSRDYRS | U33212/ |
| | cellulolyticus | MLDQIKSLGYNTIRLPYSDDILKPGTMPNSINFYQMNQDLQGLTSLQV | SEQ ID |
| | | MDKIVAYAGQIGLRIILDRHRPDCSGQSALWYTSSVSEATWISDLQAL | NO. 1 |
| | | AQRYKGNPTVVGFDLHNEPHDPACWGCGDPSIDWRLAAERAGNAVLSV | |
| | | NPNLLIFVEGVQSYNGDSYWWGGNLQGAGQYPVVLNVPNRLVYSAHDY | |
| | | ATSVYPQTWFSDPTFPNNMPGIWNKNWGYLFNQNIAPVWLGEFGTTLQ | |
| | | STTDQTWLKTLVQYLRPTAQYGADSFQWTFWSWNPDSGDTGGILKDDW | |
| | | QTVDTVKDGYLAPIKSSIFDPVG | |
| gux1 | Acidothermus | MGAPGLRRRLRAGIVSAAALGSLVSGLVAVAPVAHAAVTLKAQYKNND | YP872376/ |
| | cellulolyticus | SAPSDNQIKPGLQLVNTGSSSVDLSTVTVRYWFTRDGGSSTLVYNCDW | SEQ ID |
| | | AAMGCGNIRASFGSVNPATPTADTYLQLSFTGGTLAAGGSTGEIQNRV | NO. 2 |
| | | NKSDWSNFDETNDYSYGTNTTFQDWTKVTVYVNGVLVWGTEPSGATAS | |
| | | PSASATPSPSSSPTTSPSSSPSPSSSPTPTPSSSSPPPSSNDPYIQRF | |
| | | LTMYNKIHDPANGYFSPQGIPYHSVETLIVEAPDYGHETTSEAYSFWL | |
| | | WLEATYGAVTGNWTPFNNAWTTMETYMIPQHADQPNNASYNPNSPASY | |
| | | APEEPLPSMYPVAIDSSVPVGHDPLAAELQSTYGTPDIYGMHWLADVD | |
| | | NIYGYGDSPGGGCELGPSAKGVSYINTFQRGSQESVWETVTQPTCDNG | |
| | | KYGGAHGYVDLFIQGSTPPQWKYTDAPDADARAVQAAYWAYTWASAQG | |
| | | KASAIAPTIAKAAKLGDYLRYSLFDKYFKQVGNCYPASSCPGATGRQS | |
| | | ETYLIGWYYAWGGSSQGWAWRIGDGAAHFGYQNPLAAWAMSNVTPLIP | |
| | | LSPTAKSDWAASLQRQLEFYQWLQSAEGAIAGGATNSWNGNYGTPPAG | |
| | | DSTFYGMAYDWEPVYHDPPSNNWFGFQAWSMERVAEYYYVTGDPKAKA | |
| | | LLDKWVAWVKPNVTTGASWSIPSNLSWSGQPDTWNPSNPGTNANLHVT | |
| | | ITSSGQDVGVAAALAKTLEYYAAKSGDTASRDLAKGLLDSIWNNDQDS | |
| | | LGVSTPETRTDYSRFTQVYDPTTGDGLYIPSGWTGTMPNGDQIKPGAT | |
| | | FLSIRSWYTKDPQWSKVQAYLNGGPAPTFNYHRFWAESDFAMANADFG | |
| | | MLFPSGSPSPTPSPTPTSSPSPTPSSSPTPSPSPSPTGDTTPPSVPTG | |
| | | LQVTGTTTSSVSLSWTASTDNVGVAHYNVYRNGTLVGQPTATSFTDTG | |
| | | LAAGTSYTYTVAAVDAAGNTSAQSSPVTATTASPSPSPSPSPTPTSSP | |
| | | SPTPSPTPSPTSTSGASCTATYVVNSDWGSGFTTTVTVTNTGTRATSG | |
| | | WTVTWSFAGNQTVTNYWNTALTQSGKSVTAKNLSYNNVIQPGQSTTFG | |
| | | FNGSYSGTNTAPTLSCTASZ | |
| XylE | Acidothermus | MGHHAMRRMVTSASVVGVATLAAATVLITGGIAHAASTLKQGAEANGR | YP871941/ |
| | cellulolyticus | YFGVSASVNTLNNSAAANLVATQFDMLTPENEMKWDTVESSRGSFNFG | SEQ ID |
| | | PGDQIVAFATAHNMRVRGHNLVWHSQLPGWVSSLPLSQVQSAMESHIT | NO. 3 |
| | | AEVTHYKGKIYAWDVVNEPFDDSGNLRTDVFYQAMGAGYIADALRTAH | |
| | | AADPNAKLYLNDYNIEGINAKSDAMYNLIKQLKSQGVPIDGVGFESHF | |
| | | IVGQVPSTLQQNMQRFADLGVDVAITELDDRMPTPPSQQNLNQQATDD | |
| | | ANVVKACLAVARCVGITQWDVSDADSWVPGTFSGQGAATMFDSNLQPK | |
| | | PAFTAVLNALSASASVSPSPSPSPSPSPSPSPSPSPSPSPSPSPSPSP | |
| | | SSSPVSGGVKVQYKNNDSAPGDNQIKPGLQVVNTGSSSVDLSTVTVRY | |
| | | WFTRDGGSSTLVYNCDWAVMGCGNIRASFGSVNPATPTADTYLQLSFT | |
| | | GGTLPAGGSTGEIQSRVNKSDWSNFTETNDYSYGTNTTFQDWSKVTVY | |
| | | VNGRLVWGTEPSGTSPSPTPSPSPTPSPSPSPSPSPSPSPSPSPSPSP | |
| | | SSSPSSGCVASMRVDSSWPGGFTATVTVSNTGGVSTSGWQVGWSWPSG | |
| | | DSLVNAWNAVVSVTGTSVRAVNASYNGVIPAGGSTTFGFQANGTPGTP | |
| | | TFTCTTSADLZ | |
| aviIII | Acidothermus | MAATTQPYTWSNVAIGGGGFVDGIVFNEGAPGILYVRTDIGGMYRWDA | YP872377/ |
| | cellulolyticus | ANGRWIPLLDWVGWNNWGYNGVVSIAADPINTNKVWAAVGMYTNSWDP | SEQ ID |
| | | NDGAILRSSDQGATWQITPLPFKLGGNMPGRGMGERLAVDPNNDNILY | NO. 4 |
| | | FGAPSGKGLWRSTDSGATWSQMTNFPDVGTYIANPTDTTGYQSDIQGV | |
| | | VWVAFDKSSSSLGQASKTIFVGVADPNNPVFWSRDGGATWQAVPGAPT | |
| | | GFIPHKGVFDPVNHVLYIATSNTGGPYDGSSGDVWKFSVTSGTWTRIS | |
| | | PVPSTDTANDYFGYSGLTIDRQHPNTIMVATQISWWPDTIIFRSTDGG | |
| | | ATWTRIWDWTSYPNRSLRYVLDISAEPWLTFGVQPNPPVPSPKLGWMD | |
| | | EAMAIDPFNSDRMLYGTGATLYATNDLTKWDSGGQIHIAPMVKGLEET | |
| | | AVNDLISPPSGAPLISALGDLGGFTHADVTAVPSTIFTSPVFTTGTSV | |
| | | DYAELNPSIIVRAGSFDPSSQPNDRHVAFSTDGGKNWFQGSEPGGVTT | |
| | | GGTVAASADGSRFVWAPGDPGQPVVYAVGFGNSWAASQGVPANAQIRS | |
| | | DRVNPKTFYALSNGTFYRSTDGGVTFQPVAAGLPSSGAVGVMFHAVPG | |
| | | KEGDLWLAASSGLYHSTNGGSSWSAITGVSSAVNVGFGKSAPGSSYPA | |
| | | VFVVGTIGGVTGAYRSDDGGTTWVRINDDQHQYGNWGQAITGDPRIYG | |
| | | RVYIGTNGRGIVYGDIAGAPSGSPSPSVSPSASPSLSPSPSPSSSPSP | |
| | | SPSPSSSPSSSPSPSPSPSPSPSRSPSPSASPSPSSSPSPSSSPSSSP | |
| | | SPTPSSSPVSGGVKVQYKNNDSAPGDNQIKPGLQVVNTGSSSVDLSTV | |
| | | TVRYWFTRDGGSSTLVYNCDWAAIGCGNIRASFGSVNPATPTADTYLQ | |
| | | LSFTGGTLAAGGSTGEIQNRVNKSDWSNFTETNDYSYGTNTVFQDWSK | |
| | | VTVYVNGRLVWGTEPSGTSPSPTPSPSPTPSPSPSPSPGGDVTPPSVP | |
| | | TGVVVTGVSGSSVSLAWNASTDNVGVAHYNVYRNGVLVGQPTVTSFTD | |
| | | TGLAAGTAYTYTVAAVDAAGNTSAPSTPVTATTTSPSPSPSPTPSPTP | |
| | | SPTPSPSPSPSLSPSPSPSPSPSPSPSLSPSPSTSPSPSPSPTPSPSS | |
| | | SGVGCRATYVVNSDWGSGFTATVTVTNTGSRATSGWTVAWSFGGNQTV | |
| | | TNYWNTLLTQSGASVTATNLSYNNVIQPGQSTTFGFNATYAGTNTPPT | |
| | | PTCTTNSD | |

### A—Cellulases

Cellulases are enzyme polypeptides involved in cellulose degradation. Cellulase enzyme polypeptides are classified on the basis of their mode of action. There are two basic kinds of cellulases: the endocellulases, which cleave the polymer chains internally; and the exocellulases, which cleave from the reducing and non-reducing ends of molecules generated by the action of endocellulases. Cellulases include cellobiohydrolases, endoglucanases, and β-D-glucosidases. Endoglucanases randomly attack the amorphous regions of cellulose substrate, yielding mainly higher oligomers. Cellulobiohydrolases are exocellulases which hydrolyze crystalline cellulose and release cellobiose (glucose dimer). Both types of enzymes hydrolyze β-1,4-glycosidic bonds. β-D-glucosidases or cellulobiase converts oligosaccharides and cellubiose to glucose.

According to the present invention, plants may be engineered to comprise a gene encoding a cellulase enzyme polypeptide. Alternatively, plants may be engineered to comprise more than one gene encoding a cellulase enzyme polypeptide. For example, plants may be engineered to comprise one or more genes encoding a cellulase of the cellubiohydrolase class, one or more genes encoding a cellulase of the endoglucanase class, and/or one or more genes encoding a cellulase of the β-D-glucosidase class.

Examples of endoglucanase genes that can be used in the present invention can be obtained from Aspergillus aculeatus (U.S. Pat. No. 6,623,949; WO 94/14953), Aspergillus kawachii (U.S. Pat. No. 6,623,949), Aspergillus oryzae (Kitamoto et al., Appl. Microbiol. Biotechnol., 1996, 46: 538-544; U.S. Pat. No. 6,635,465), Aspergillus nidulans (Lockington et al., Fungal Genet. Biol., 2002, 37: 190-196), Cellulomonas fimi (Wong et al., Gene, 1986, 44: 315-324), Bacillus subtilis (MacKay et al., Nucleic Acids Res., 1986, 14: 9159-9170), Cellulomonas pachnodae (Cazemier et al., Appl. Microbiol. Biotechnol., 1999, 52: 232-239), Fusarium equiseti (Goedegebuur et al., Curr. Genet., 2002, 41: 89-98), Fusarium oxysporum (Hagen et al., Gene, 1994, 150: 163-167; Sheppard et al., Gene, 1994, 150: 163-167), Humicola insolens (U.S. Pat. No. 5,912,157; Davies et al., Biochem J., 2000, 348: 201-207), Hypocrea jecorina (Penttila et al., Gene, 1986, 45: 253-263), Humicola grisea (Goedegebuur et al., Curr. Genet., 2002, 41: 89-98), Micromonospora cellulolyticum (Lin et al., J. Ind. Microbiol., 1994, 13: 344-350), Myceliophthora thermophila (U.S. Pat. No. 5,912,157), Rhizopus oryzae (Moriya et al., J. Bacteriol., 2003, 185: 1749-1756), Trichoderma reesei (Saloheimo et al., Mol. Microbiol., 1994, 13: 219-228), and Trichoderma viride (Kwon et al., Biosci. Biotechnol. Biochem., 1999, 63: 1714-1720; Goedegebuur et al., Curr. Genet., 2002, 41: 89-98).

In certain embodiments, plants are engineered to comprise the endo-1,4-β-glucanase E1 gene (GenBank Accession No. U33212, See Table 1). This gene was isolated from the thermophilic bacterium Acidothermus cellulolyticus. Acidothermus cellulolyticus has been characterized with the ability to hydrolyze and degrade plant cellulose. The cellulase complex produced by A. cellulolyticus is known to contain several different thermostable cellulase enzymes with maximal activities at temperatures of 75° C. to 83° C. These cellulases are resistant to inhibition from cellobiose, an end product of the reactions catalyzed by endo- and exo-cellulases.

The E1 endo-1,4-β-glucanase is described in detail in U.S. Pat. No. 5,275,944. This endoglucanase demonstrates a temperature optimum of 83° C. and a specific activity of 40 μmol glucose release from carboxymethylcellulose/min/mg protein. This E1 endoglucanase was further identified as having an isoelectric pH of 6.7 and a molecular weight of 81,000 Daltons by SDS polyacrylamide gel electrophoresis. It is synthesized as a precursor with a signal peptide that directs it to the export pathway in bacteria. The mature enzyme polypeptide is 521 amino acids (aa) in length. The crystal structure of the catalytic domain of about 40 kD (358 aa) has been described (J. Sakon et al., Biochem., 1996, 35: 10648-10660). Its pro/thr/ser-rich linker is 60 aa, and the cellulose binding domain (CBD) is 104 aa. The properties of the cellulose binding domain that confer its function are not well-characterized. Plant expression of the E1 gene has been reported (see for example, M. T. Ziegler et al., Mol. Breeding, 2000, 6: 37-46; Z. Dai et al., Mol. Breeding, 2000, 6: 277-285; Z. Dai et al., Transg. Res., 2000, 9: 43-54; and T. Ziegelhoffer et al., Mol. Breeding, 2001, 8: 147-158).

Examples of cellobiohydrolase genes that can be used in the present invention can be obtained from Acidothermus cellulolyticus, Acremonium cellulolyticus (U.S. Pat. No. 6,127,160), Agaricus bisporus (Chow et al., Appl. Environ. Microbiol., 1994, 60: 2779-2785), Aspergillus aculeatus (Takada et al., J. Ferment. Bioeng., 1998, 85: 1-9), Aspergillus niger (Gielkens et al., Appl. Environ. Microbiol., 65: 1999, 4340-4345), Aspergillus oryzae (Kitamoto et al., Appl. Microbiol. Biotechnol., 1996, 46: 538-544), Athelia rolfsii (EMBL accession No. AB103461), Chaetomium thermophilum (EMBL accession Nos. AX657571 and CQ838150), Cullulomonas fimi (Meinke et al., Mol. Microbiol., 1994, 12: 413-422), Emericella nidulans (Lockington et al., Fungal Genet. Biol., 2002, 37: 190-196), Fusarium oxysporum (Hagen et al., Gene, 1994, 150: 163-167), Geotrichum sp. 128 (EMBL accession No. AB089343), Humicola grisea (de Oliviera and Radford, Nucleic Acids Res., 1990, 18: 668; Takashima et al., J. Biochem., 1998, 124: 717-725), Humicola nigrescens (EMBL accession No. AX657571), Hypocrea koningii (Teeri et al., Gene, 1987, 51: 43-52), Mycelioptera thermophila (EMBL accession No. AX657599), Neocallimastix patriciarum (Denman et al., Appl. Environ. Microbiol., 1996, 62: 1889-1896), Phanerochaete chrysosporium (Tempelaars et al., Appl. Environ. Microbiol., 1994, 60: 4387-4393), Thermobifida fusca (Zhang, Biochemistry, 1995, 34: 3386-3395), Trichoderma reesei (Terri et al., BioTechnology, 1983, 1: 696-699; Chen et al., BioTechnology, 1987, 5: 274-278), and Trichoderma viride (EMBL accession Nos. A4368686 and A4368688).

Examples of β-D-glucosidase genes that can be used in the present invention can be obtained from Aspergillus aculeatus (Kawaguchi et al., Gene, 1996, 173: 287-288), Aspergillus kawachi (Iwashita et al., Appl. Environ. Microbiol., 1999, 65: 5546-5553), Aspergillus oryzae (WO 2002/095014), Cellulomonas biazotea (Wong et al., Gene, 1998, 207: 79-86), Penicillium funiculosum (WO 200478919), Saccharomycopsis fibuligera (Machida et al., Appl. Environ. Microbiol., 1988, 54: 3147-3155), Schizosaccharomyces pombe (Wood et al., Nature, 2002, 415: 871-880), and Trichoderma reesei (Barnett et al., BioTechnology, 1991, 9: 562-567).

Other examples of cellulases that can be used in accordance with the present invention include family 48 glycoside hydrolases such as guxl from Acidothermus cellulolyticus, avicelases such as aviIII from Acidothermus cellulolyticus, and cbhE from Talaromyces emersonii. (See Table 1.)

Transgene expression of cellulases in plants for the conversion of cellulose to glucose has been reported (see, for example, Y. Jin Cai et al., Appl. Environ. Microbiol., 1999, 65: 553-559; C. R. Sanchez et al., Revista de Microbiologica, 1999, 30: 310-314; R. Cohen et al., Appl. Environ., 2995, 71: 2412-2417; Z. Dai et al., Transg. Res., 2005, 14: 627-543).

### B—Hemicellulases

Hemicellulases are enzyme polypeptides that are involved in hemicellulose degradation. Hemicellulases include xylanases, arabinofuranosidases, acetyl xylan esterases, ferulic acid esterases, xyloglucanases, β-glucanases, β-xylosidases, glucuronidases, mannanases, galactanases, and arabinases. Similar to cellulase enzyme polypeptides, hemicellulases are classified on the basis of their mode of action: the endo-acting hemicellulases attack internal bonds within the polysaccharide chain; the exo-acting hemicellulases act progressively from either the reducing or non-reducing end of polysaccharide chains.

Exemplary xylanases include, for example Xyn Z, whose sequence is shown below: (SEQ ID NO: 5):

MSRKLFSVLLVGLMLMTSLLVTISSTSAASLPTMPPSGYDQVRNGVPRGQ
VVNISYFSTATNSTRPARVYLPPGYSKDKKYSVLYLLHGIGGSENDWFEG
GGRANVIADNLIAEGKIKPLIIVTPNTNAAGPGIADGYENFTKDLLNSLI
PYIESNYSVYTDREHRAIAGLSMGGGQSFNIGLTNLDKFAYIGPISAAPN
TYPNERLFPDGGKAAREKLKLLFIACGTNDSLIGFGQRVHEYCVANNINH
VYWLIQGGGHDFNVWKPGLWNFLQMADEAGLTRDGNTPVPTPSPKPANTR
IEAEDYDGINSSSIEIIGVPPEGGRGIGYITSGDYLVYKSIDFGNGATSF
KAKVANANTSNIELRLNGPNGTLIGTLSVKSTGDWNTYEEQTCSISKVTG
INDLYLVFKGPVNIDWFTFGVESSSTGLGDLNGDGNINSSDLQALKRHLL
GISPLTGEALLRADVNRSGKVDSTDYSVLKRYILRIITEFPGQGDVQTPN
PSVTPTQTPIPTISGNALRDYAEARGIKIGTCVNYPFYNNSDPTYNSILQ
REFSMVVCENEMKFDALQPRQNVFDFSKGDQLLAFAERNGMQMRGHTLIW
HNQNPSWLTNGNWNRDSLLAVMKNHITTVMTHYKGKIVEWDVANECMDDS
GNGLRSSIWRNVIGQDYLDYAFRYAREADPDALLFYNDYNIEDLGPKSNA
VFNMIKSMKERGVPIDGVGFQCHFINGMSPEYLASIDQNIKRYAEIGVIV
SFTEIDIRIPQSENPATAFQVQANNYKELMKICLANPNCNTFVMWGFTDK
YTWIPGTFPGYGNPLIYDSNYNPKPAYNAIKEALMGY

According to the present invention, plants may be engineered to comprise a gene encoding a hemicellulase enzyme polypeptide. Alternatively, plants may be engineered to comprise more than one gene encoding a hemicellulase enzyme polypeptide. For example, plants may be engineered to comprise one or more genes encoding a hemicellulase of the xylanase class, one or more genes encoding a hemicellulase of the arabinofuranosidase class, one or more genes encoding a hemicellulase of the acetyl xylan esterase class, one or more genes encoding a hemicellulase of the glucuronidase class, one or more genes encoding a hemicellulase of the mannanase class, one or more genes encoding a hemicellulase of the galactanase class, and/or one or more genes encoding a hemicellulase of the arabinase class.

Examples of endo-acting hemicellulases include endoarabinanase, endoarabinogalactanase, endoglucanase, endomannanase, endoxylanase, and feraxan endoxylanase. Examples of exo-acting hemicellulases include α-L-arabinosidase, β-L-arabinosidase, α-1,2-L-fucosidase, α-D-galactosidase, β-D-galactosidase, β-D-glucosidase, β-D-glucuronidase, β-D-mannosidase, β-D-xylosidase, exo-glucosidase, exo-mannobiohydrolase, exo-mannanase, exo-xylanase, xylan α-glucuronidase, and coniferin β-glucosidase.

Hemicellulase genes can be obtained from any suitable source, including fungal and bacterial organisms, such as Aspergillus, Disporotrichum, Penicillium, Neurospora, Fusarium, Trichoderma, Humicola, Thermomyces, and Bacillus. Examples of hemicellulases that can be used in the present invention can be obtained from Acidothermus cellulolyticus, Acidobacterium capsulatum (Inagaki et al., Biosci. Biotechnol. Biochem., 1998, 62: 1061-1067), Agaricus bisporus (De Groot et al., J. Mol. Biol., 1998, 277: 273-284), Aspergillus aculeatus (U.S. Pat. No. 6,197,564; U.S. Pat. No. 5,693,518), Aspergillus kawachii (Ito et al., Biosci. Biotechnol. Biochem., 1992, 56: 906-912), Aspergillus niger (EMBL accession No. AF108944), Magnaporthe grisea (Wu et al., Mol. Plant Microbe Interact., 1995, 8: 506-514), Penicillium chrysogenum (Haas et al., Gene, 1993, 126: 237-242), Talaromyces emersonii (WO 02/24926), and Trichoderma reesei (EMBL accession Nos. X69573, X69574, and AY281369).

In certain embodiments, plants are engineered to comprise the A. cellulolyticus endoxylanase xylE (see the Examples section).

### C—Ligninases

Ligninases are enzyme polypeptides that are involved in the degradation of lignin. Lignin-degrading enzyme polypeptides include, but are not limited to, lignin peroxidases, manganese-dependent peroxidases, hybrid peroxidases (which exhibit combined properties of lignin peroxidases and manganese-dependent peroxidases), and laccases. Hydrogen peroxide, required as co-substrate by the peroxidases, can be generated by glucose oxidase, aryl alcohol oxidase, and/or lignin peroxidase-activated glyoxal oxidase.

According to the present invention, plants may be engineered to comprise a gene encoding a ligninase enzyme polypeptide. Alternatively, plants may be engineered to comprise more than one gene encoding a ligninase enzyme polypeptide. For example, plants may be engineered to comprise one or more genes encoding a ligninase of the lignin peroxidase class, one or more genes encoding a ligninase of the manganese-dependent peroxidase class, one or more genes encoding a ligninase of the hybrid peroxidase class, and/or one or more genes encoding a ligninase of the laccase class.

Lignin-degrading genes may be obtained from Acidothermus cellulolyticus, Bjerkandera adusta, Ceriporiopsis subvermispora (see WO 02/079400), Coprinus cinereus, Coriolus hirsutus, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride.

Examples of genes encoding ligninases that can be used in the invention can be obtained from Bjerkandera adusta (WO 2001/098469), Ceriporiopsis subvermispora (Conesa et al., J. Biotechnol., 2002, 93: 143-158), Cantharellus cibariusi (Ng et al., Biochem. and Biophys. Res. Comm., 2004, 313: 37-41), Coprinus cinereus (WO 97/008325; Conesa et al., J. Biotechnol., 2002, 93: 143-158), Lentinula edodes (Nagai et al., Applied Microbiol. and Biotechnol., 2002, 60: 327-335, 2002), Melanocarpus albomyces (Kiiskinen et al., FEBS Letters, 2004, 576: 251-255, 2004), Myceliophthora thermophila (WO 95/006815), Phanerochaete chrysosporium (Conesa et al., J. Biotechnol., 2002, 93: 143-158; Martinez, Enz, Microb, Technol, 2002, 30: 425-444), Phlebia radiata (Conesa et al., J. Biotechnol., 2002, 93: 143-158), Pleurotus eryngii (Conesa et al., J. Biotechnol., 2002, 93: 143-158), Polyporus pinsitus (WO 96/000290), Rigidoporus lignosus (Garavaglia et al., J. of Mol. Biol., 2004, 342: 1519-1531), Rhizoctonia solani (WO 96/007988), Scytalidium thermophilum (WO 95/033837), Tricholoma giganteum (Wang et al., Biochem. Biophys. Res. Comm., 2004, 315: 450-454), and Trametes versicolor (Conesa et al., J. Biotechnol., 2002, 93: 143-158).

For example, plants may be engineered to comprise one or more lignin peroxidases. Genes encoding lignin peroxidases may be obtained from Phanerochaete chrysosporium or Phlebia radiata. Lignin-peroxidases are glycosylated heme proteins (MW 38 to 46 kDa) which are dependent on hydrogen peroxide for activity and catalyze the oxidative cleavage of lignin polymer. At least six (6) heme proteins (H1, H2, H6, H7, H8 and H10) with lignin peroxidase activity have been identified Phanerochaete chrysosporium in strain BKMF-1767. In certain embodiments, plants are engineered to comprise the white rot filamentous Phanerochaete chrysosporium ligninase (CGL5) (H.A. de Boer et al., Gene, 1988, 69(2): 369) (see the Examples section).

### D—Other Lignocellulolytic Enzyme Polypeptides

In addition to cellulases, hemicellulases and ligninases, lignocellulolytic enzyme polypeptides that can be used in the practice of the present invention also include enzymes that degrade pectic substances or phenolic acids such as ferulic acid. Pectic substances are composed of homogalacturonan (or pectin), rhamno-galacturonan, and xylogalacturonan. Enzymes that degrade homogalacturonan include pectate lyase, pectin lyase, polygalacturonase, pectin acetyl esterase, and pectin methyl esterase. Enzymes that degrade rhamnogalacturonan include alpha-arabinofuranosidase, beta-galactosidase, galactanase, arabinanase, alpha-arabinofuranosidase, rhamnogalacturonase, rhamnogalacturonan lyase, and rhamnogalacturonan acetyl esterase. Enzymes that degrade xylogalacturonan include xylogalacturonosidase, xylogalacturonase, and rhamnogalacturonan lyase.

Phenolic acids include ferulic acid, which functions in the plant cell wall to cross-link cell wall components together. For example, ferulic acid may cross-link lignin to hemicellulose, cellulose to lignin, and/or hemicellulose polymers to each other. Ferulic acid esterases cleave ferulic acid, disrupting the cross linkages.

Other enzymes that may enhance or promote lignocellulose disruption and/or degradation include, but are not limited to, amylases (e.g., alpha amylase and glucoamylase), isomerases (e.g., arabinose isomerase and xylose isomerase), esterases, lipases, phospholipases, phytases, proteases, and peroxidases.

### E—Combinations of Lignocellulolytic Enzyme Polypeptides

According to the present invention, plants may be engineered to comprise a gene encoding a lignocellulolytic enzyme polypeptide, e.g., a cellulase enzyme polypeptide, a hemicellulase enzyme polypeptide, or a ligninase enzyme polypeptide. Alternatively, plants may be engineered to comprise two or more genes encoding lignocellulolytic enzyme polypeptides, e.g., enzymes from different classes of cellulases, enzymes from different classes of hemicellulases, enzymes from different classes of ligninases, or any combinations thereof. For example, combinations of genes may be selected to provide efficient degradation of one component of lignocellulose (e.g., cellulose, hemicellulose, or lignin). Alternatively, combinations of genes may be selected to provide efficient degradation of the lignocellulosic material.

In certain embodiments, genes are optimized for the substrate (e.g., cellulose, hemicellulase, lignin or whole lignocellulosic material) in a particular plant (e.g., corn, tobacco, switchgrass). Tissue from one plant species is likely to be physically and/or chemically different from tissue from another plant species. Selection of genes or combinations of genes to achieve efficient degradation of a given plant tissue is within the skill of artisans in the art.

In some embodiments, combinations of genes are selected to provide for synergistic enzymes activity (i.e., genes are selected such that the interaction between distinguishable enzymes or enzyme activities results in the total activity of the enzymes taken together being greater than the sum of the effects of the individual activities).

Efficient lignocellulolytic activity may be achieved by production of two or more enzymes in a single transgenic plant. As mentioned above, plants may be transformed to express more than one enzyme, for example, by employing the use of multiple gene constructs encoding each of the selected enzymes or a single construct comprising multiple nucleotide sequences encoding each of the selected enzymes. Alternatively, individual transgenic plants, each stably transformed to express a given enzyme, may be crossed by methods known in the art (e.g., pollination, hand detassling, cytoplasmic male sterility, and the like) to obtain a resulting plant that can produce all the enzymes of the individual starting plants.

Alternatively or additionally, efficient lignocellulolytic activity may be achieved by production of two or more lignocellulolytic enzyme polypeptides in separate plants. For example, three separate lines of plants (e.g., corn), one expressing one or more enzymes of the cellulase class, another expressing one or more enzymes of the hemicellulase class and the third one expressing one or more enzymes of the ligninase class, may be developed and grown simultaneously. The desired “blend” of enzymes produced may be achieved by simply changing the seed ratio, taking into account farm climate and soil type, which are expected to influence enzyme yields in plants.

Other advantages of this approach include, but are not limited to, increased plant health (which is known to be adversely affected as the number of introduced genes increases), simpler transformations procedures and great flexibility in incorporating the desired traits in commercial plant varieties for large-scale production.

### G—Thermophilic and Thermostable Enzyme Polypeptides

It may be sometimes desirable to use transgenic plants expressing thermophilic and/or thermostable enzyme polypeptides. For example, enzyme polypeptides whose optimal range of temperature for activity (thermophilic enzyme polypeptides) may be expressed in transgenic plants in accordance with the invention. Without wishing to be bound by any particular theory, the limited activity or absence of activity during growth of the plant (at moderate or low temperatures, at which the enzyme polypeptide is less active) may be beneficial to the health of the plant. Alternatively or additionally, and without wishing to be bound by any particular theory, such enzyme polypeptides may facilitate increased hydrolysis because of their high activity at high temperature conditions commonly used in the processing of cellulosic biomass.

In some embodiments, the present invention provides a transgenic plant, the genome of which is augmented with a recombinant polynucleotide encoding at least one lignocellulolytic enzyme polypeptide that exhibits low activity at a temperature below about 60° C., below about 50° C., below about 40° C., or below about 30° C. In some embodiments, the present invention provides a transgenic plant, the genome of which is augmented with a recombinant polynucleotide encoding at least one lignocellulolytic enzyme polypeptide that exhibits high activity at a temperature above about 50° C., above about 60° C., above about 70° C., above about 80° C., or above about 90° C.

In some embodiments, the present invention provides a transgenic plant, the genome of which is augmented with a recombinant polynucleotide encoding at least one lignocellulolytic enzyme polypeptide that is or is homologous to a lignocellulolytic enzyme polypeptide found in a thermophilic microorganism (e.g., bacterium, fungus, etc.). In some such embodiments, the thermophilic organism is a bacterium that is a member of a genus selected from the group consisting of Aeropyrum, Acidilobus, Acidothermus, Aciduliprofundum, Anaerocellum, Archaeoglobus, Aspergillus, Bacillus, Caldibacillus, Caldicellulosiruptor, Caldithrix, Cellulomonas, Chaetomium, Chloroflexus, Clostridium, Cyanidium, Deferribacter, Desulfotomaculum, Desulfurella, Desulfurococcus, Fervidobacterium, Geobacillus, Geothermobacterium, Humicola, Ignicoccus, Marinitoga, Methanocaldococcus, Methanococcus, Methanopyrus, Methanosarcina, Methanothermobacter, Nautilia, Pyrobaculum, Pyrococcus, Pyrodictium, Rhizomucor, Rhodothermus, Staphylothermus, Scylatidium, Spirochaeta, Sulfolobus, Talaromyces, Thermoascus, Thermobifida, Thermococcus, Thermodesulfobacterium, Thermodesulfovibrio, Thermomicrobium, Thermoplasma, Thermoproteus, Thermothrix, Thermotoga, Thermus, and Thiobacillus; in some such embodiments, the thermophilic microorganism is a bacterium that is a member of a species selected from the group consisting of Acidothermus cellulolyticus, Pyrococcus furiosus, and Talaromyces emersonii.

### II. Nucleic Acid Constructs

Nucleic acid constructs to be used in the practice of the present invention generally encompass expression cassettes for expression in the plant of interest. The cassette generally includes 5′ and 3′ regulatory sequences operably linked to a nucleotide sequence encoding a lignocellulolytic enzyme polypeptide (e.g., a cellulase, a hemicellulase or ligninase).

### Expression Cassettes

Techniques used to isolate or clone a gene encoding an enzyme (e.g., a lignocellulolytic enzyme polypeptide) are known in the art and include isolation from genomic DNA, preparation from cDNA, or a combination thereof. The cloning of a gene from such genomic DNA, can be effected, e.g., by using polymerase chain reaction (PCR) or antibody screening or expression libraries to detect cloned DNA fragments with shared structural features (Innis et al., “PCR: A Guide to Method and Application”, 1990, Academic Press: New York). Other nucleic acid amplification procedures such as ligase chain reaction (LCR), ligated activated transcription (LAT) and nucleotide sequence-based amplification (NASBA) may be used.

The expression cassette will generally include in the 5′-3′ direction of transcription, a transcriptional and translational initiation region, a coding sequence for a lignocellulolytic enzyme polypeptide, and a transcriptional and translational termination region functional in plants. The transcriptional initiation region, i.e., the promoter, can be native or analogous (i.e., found in the native plant) or foreign or heterologous (i.e., not found in the native plant) to the plant host. Additionally, the promoter can be the natural sequence or alternatively a synthetic sequence.

Expression of some enzyme polypeptides, such as those that are required in large quantity and can be produced and stored during the entire growth of the plant, may be better accomplished using constitutive promoters. Production of some enzyme polypeptides, such as those that have deleterious effects on phenotype and would therefore be better expressed during senescence or after harvest, may be better accomplished using inducible promoters. With stacked-trait enzyme polypeptides in plants, it may be desirable to have a combination of constitutive and inducible promoters driving the genes of interest.

In certain embodiments, the promoter is a constitutive plant promoter, i.e., an unregulated promoter that allows continual expression of a gene associated with it. Examples of plant promoters include, but are not limited to, the 35S cauliflower mosaic virus (CaMV) promoter, a promoter of nopaline synthase, and a promoter of octopine synthase. Examples of other constitutive promoters used in plants are the 19S promoter and promoters from genes encoding actin and ubiquitin. Promoters may be obtained from genomic DNA by using polymerase chain reaction (PCR), and then cloned into the construct.

The constitutive promoter may allow expression of an associated gene throughout the life of a plant. In some embodiments, the lignocellulolytic enzyme polypeptide is produced throughout the life of the plant. In some embodiments, the lignocellulolytic enzyme polypeptide is active through the life of the plant. Alternatively or additionally, a constitutive promoter may allow expression of an associated gene in all or a majority of plant tissues. In some embodiments, the lignocellulolytic enzyme polypeptide is present in all plant tissues during the life of the plant.

In some embodiments, the promoter is an inducible promoter. Examples of inducible promoters that may be used in accordance with the invention include, but are not limited to, the alcA promoter from Aspergillus nidulans (which is responsive to ethanol) and regulatory elements from the mammalian glucocorticoid receptor that have been engineered to respond to RH5992 (a non-steroidal ecdysone agonist that can be used as a lepidopteran control agent on a variety of crops).

Other sequences that can be present in nucleic acid constructs are sequences that enhance gene expression such as intron sequences and leader sequences. Examples of introns that have been reported to enhance expression include, but are not limited to, the introns of the Maize Adh1 gene and introns of the Maize bronze1 gene (J. Callis et. al., Genes Develop. 1987, 1: 1183-1200). Examples of non-translated leader sequences that are known to enhance expression include, but are not limited to, leader sequences from Tobacco Mosaic Virus (TMV, the “omegasequence”), Maize Chlorotic Mottle Virus (MCMV), and Alfalfa Mosaic Virus (A1MV) (see, for example, D. R. Gallie et al., Nucl. Acids Res. 1987, 15: 8693-8711; J. M. Skuzeski et. al., Plant Mol. Biol. 1990, 15: 65-79).

The transcriptional and translational termination region can be native with the transcription initiation region, can be native with the operably linked polynucleotide sequence of interest, or can be derived from another source. Convenient termination regions are available from the T₁-plasmid of A. tumefaciens, such as the octopine synthase and nopaline synthase termination regions (An et al., Plant Cell, 1989, 1: 115-122; Guerineau et al., Mol. Gen. Genet. 1991, 262: 141-144; Proudfoot, Cell, 1991, 64: 671-674; Sanfacon et al., Genes Dev. 1991, 5: 141-149; Mogen et al., Plant Cell, 1990, 2:1261-1272; Munroe et al., Gene, 1990, 91:151-158; Ballas et al., Nucleic Acids Res., 1989, 17: 7891-7903; and Joshi et al., Nucleic Acid Res., 1987, 15: 9627-9639).

Where appropriate, the gene(s) or polynucleotide sequence(s) encoding the enzyme(s) of interest may be modified to include codons that are optimized for expression in the transformed plant (Campbell and Gowri, Plant Physiol., 1990, 92: 1-11; Murray et al., Nucleic Acids Res., 1989, 17: 477-498; Wada et al., Nucl. Acids Res., 1990, 18: 2367, and U.S. Pat. Nos. 5,096,825; 5,380,831; 5,436,391; 5,625,136, 5,670,356 and 5,874,304). Codon optimized sequences are synthetic sequences, and preferably encode the identical polypeptide (or an enzymatically active fragment of a full length polypeptide which has substantially the same activity as the full length polypeptide) encoded by the non-codon optimized parent polynucleotide which encodes a lignocellulolytic enzyme polypeptide.

### Other Polynucleotide Sequence

Optional components of nucleic acid constructs include one or more marker genes. Marker genes are genes that impart a distinct phenotype to cells expressing the marker gene and thus allow transformed cells to be distinguished from cells that do not have the marker. Such genes may encode either a selectable or screenable marker. The characteristic phenotype allows the identification of cells, groups of cells, tissues, organs, plant parts or whole plants containing the construct. Many examples of suitable marker genes are known in the art. The marker may also confer additional benefit(s) to the transgenic plant such as herbicide resistance, insect resistance, disease resistance, and increased tolerance to environmental stress (e.g., drought).

Alternatively, a marker gene can provide some other visibly reactive response (e.g., may cause a distinctive appearance such as color or growth pattern relative to plants or plant cells not expressing the selectable marker gene in the presence of some substance, either as applied directly to the plant or plant cells or as present in the plant or plant cell growth media). It is now well known in the art that transcriptional activators of anthocyanin biosynthesis, operably linked to a suitable promoter in a construct, have widespread utility as non-phytotoxic markers for plant cell transformation.

Examples of markers that provide resistance to herbicides include, but are not limited to, the bar gene from Streptomyces hygroscopicus encoding phosphinothricin acetylase (PAT), which confers resistance to the herbicide glufosinate; mutant genes which encode resistance to imidazalinone or sulfonylurea such as genes encoding mutant form of the ALS and AHAS enzyme (Lee at al., EMBO J., 1988, 7: 1241; Miki et al., Theor. Appl. Genet., 1990, 80: 449; and U.S. Pat. No. 5,773,702); genes which confer resistance to glycophosphate such as mutant forms of EPSP synthase and aroA; resistance to L-phosphinothricin such as the glutamine synthetase genes; resistance to glufosinate such as the phosphinothricin acetyl transferase (PAT and bar) gene; and resistance to phenoxy propionic acids and cyclohexones such as the ACCAse inhibitor-encoding genes (Marshall et al., Theor. Appl. Genet., 1992, 83: 435).

Examples of genes which confer resistance to pests or disease include, but are not limited to, genes encoding a Bacillus thuringiensis protein such as the delta-endotoxin (U.S. Pat. No. 6,100,456); genes encoding lectins (Van Damme et al., Plant Mol. Biol., 1994, 24: 825); genes encoding vitamin-binding proteins such as avidin and avidin homologs which can be used as larvicides against insect pests; genes encoding protease or amylase inhibitors, such as the rice cysteine proteinase inhibitor (Abe et al., J. Biol. Chem., 1987, 262: 16793) and the tobacco proteinase inhibitor I (Hubb et al., Plant Mol. Biol., 1993, 21: 985); genes encoding insect-specific hormones or pheromones such as ecdysteroid and juvenile hormone, and variants thereof, mimetics based thereon, or an antagonists or agonists thereof; genes encoding insect-specific peptides or neuropeptides which, upon expression, disrupts the physiology of the pest; genes encoding insect-specific venom such as that produced by a wasp, snake, etc.; genes encoding enzymes responsible for the accumulation of monoterpenes, sesquiterpenes, asteroid, hydroxamic acid, phenylpropanoid derivative or other non-protein molecule with insecticidal activity; genes encoding enzymes involved in the modification of a biologically active molecule (U.S. Pat. No. 5,539,095); genes encoding peptides which stimulate signal transduction; genes encoding hydrophobic moment peptides such as derivatives of Tachyplesin which inhibit fungal pathogens; genes encoding a membrane permease, a channel former or channel blocker (Jaynes et al., Plant Sci., 1993, 89: 43); genes encoding a viral invasive protein or complex toxin derived therefrom (Beachy et al., Ann. Rev. Phytopathol., 1990, 28: 451); genes encoding an insect-specific antibody or antitoxin or a virus-specific antibody (Tavladoraki et al., Nature, 1993, 366: 469); and genes encoding a developmental-arrestive protein produced by a plant, pathogen or parasite which prevents disease.

Examples of genes which confer resistance to environmental stress include, but are not limited to, mtld and HVA1, which are genes that confer resistance to environmental stress factors; rd29A and rd19B, which are genes of Arabidopsis thaliana that encode hydrophilic proteins which are induced in response to dehydration, low temperature, salt stress, or exposure to abscisic acid and enable the plant to tolerate the stress (Yamaguchi-Shinozaki et al., Plant Cell, 1994, 6: 251-264). Other genes contemplated can be found in U.S. Pat. Nos. 5,296,462 and 5,356,816.

### Affinity Tags

In certain embodiments, lignocelluloytic enzyme polypeptides are fused to one or more affinity tags. Such affinity tags may facilitate, for example, purification of the lignocellulolytic enzyme polypeptides. Exemplary affinity tags include, but are not limited to, HAT (histidine affinity tag), FLAG (typically a peptide having the sequence N-DYKDDDDK-C (SEQ ID NO:6)), c-myc, hemaglutinin antingen, and His (such as a poly-histidine tag).

### Tissue-Specific Expression

In certain embodiments, lignocellulolytic enzyme polypeptide expression is targeted to specific tissues of the transgenic plant such that the lignocellulolytic enzyme is present in only some plant tissues during the life of the plant. For example, tissue specific expression may be performed to preferentially express enzymes in leaves and stems rather than grain or seed (which can reduce concerns about human consumption of genetically modified organism (GMOs)). Tissue-specific expression has other benefits including targeted expression of enzyme(s) to the appropriate substrate.

Tissue specific expression may be functionally accomplished by introducing a constitutively expressed gene in combination with an antisense gene that is expressed only in those tissues where the gene product (e.g., lignocellulolytic enzyme polypeptide) is not desired. For example, a gene coding for a lignocellulolytic enzyme polypeptide may be introduced such that it is expression in all tissues using the 35S promoter from Cauliflower Mosaic Virus. Expression of an antisense transcript of the gene in maize kernel, using for example a zein promoter, would prevent accumulation of the lignocellulolytic enzyme polypeptide in seed. Hence the enzyme encoded by the introduced gene would be present in all tissues except the kernel.

Moreover, several tissue-specific regulated genes and/or promoters have been reported in plants. Some reported tissue-specific genes include the genes encoding the seed storage proteins (such as napin, cruciferin, β-conglycinin, and phaseolin) zein or oil body proteins (such as oleosin), or genes involved in fatty acid biosynthesis (including acyl carrier protein, stearoyl-ACP desaturase, and fatty acid desaturases (fad 2-1)), and other genes expressed during embryo development, such as Bce4 (Kridl et al., Seed Science Research, 1991, 1: 209). Examples of tissue-specific promoters, which have been described include the lectin (Vodkin, Prog. Clin. Biol. Res., 1983, 138: 87; Lindstrom et al., Der. Genet., 1990, 11: 160), corn alcohol dehydrogenase 1 (Dennis et al., Nucleic Acids Res., 1984, 12: 983), corn light harvesting complex (Bansal et al., Proc. Natl. Acad. Sci. USA, 1992, 89: 3654), corn heat shock protein, pea small subunit RuBP carboxylase, Ti plasmid mannopine synthase, Ti plasmid nopaline synthase, petunia chalcone isomerase (van Tunen et al., EMBO J., 1988, 7:125), bean glycine rich protein 1 (Keller et al., Genes Dev., 1989, 3: 1639), truncated CaMV 35s (Odell et al., Nature, 1985, 313: 810), potato patatin (Wenzler et al., Plant Mol. Biol., 1989, 13: 347), root cell (Yamamoto et al., Nucleic Acids Res., 1990, 18: 7449), maize zein (Reina et al., Nucleic Acids Res., 1990, 18: 6425; Kriz et al., Mol. Gen. Genet., 1987, 207: 90; Wandelt et al., Nucleic Acids Res., 1989, 17 2354), PEPCase, R gene complex-associated promoters (Chandler et al., Plant Cell, 1989, 1: 1175), and chalcone synthase promoters (Franken et al., EMBO J., 1991, 10: 2605). Particularly useful for seed-specific expression is the pea vicilin promoter (Czako et al., Mol. Gen. Genet., 1992, 235: 33).

### Subcellular Specific Expression

In some embodiments, lignocellulolytic enzyme polypeptide expression is targeted to specific cellular compartments or organelles, such as, for example, the cytosol, the vacuole, the nucleus, the endoplasmic reticulum, the cell wall, the mitochondria, the apoplast, the peroxisomes, plastids, or combinations thereof. In some embodiments of the invention, the lignocellulolytic enzyme polypeptide is expressed in one or more subcellular compartments or organelles, for example, the cell wall and/or endoplasmic reticulum, during the life of the plant.

Directing the lignocellulolytic enzyme polypeptide to a specific cell compartment or organelle may allow the enzyme to be localized such that it will not come into contact with the substrate during plant growth. The enzyme would not act until it is allowed to contact its substrate, e.g., following physical disruption of the cell integrity by milling.

Targeting expression of a lignocellulolytic enzyme polypeptide to the cell wall (as in the apoplast) can help overcome the difficulty of mixing hydrophobic cellulose and hydrophilic enzymes that make it hard to achieve efficient hydrolysis with external enzymes.

In some embodiments, the invention provides plants engineered to express a lignocellulolytic enzyme polypeptide (or more than one lignocellulolytic enzyme polypeptide) in more than one subcellular compartments or organelles. By using promoters targeted at different locations in the plant cell, one can increase the total enzyme produced in the plant. Thus, for example, using an apoplast promoter with the E1 gene, and a chloroplast promoter with the E1 gene, in a plant would increase total production of E1 compared to a single promoter/E1 construct in the plant. Furthermore, by using promoters targeted at different locations in the plant in the case of expression of multiple lignocellulolytic enzyme polypeptides, one can minimize in vivo (pre-processing) deconstruction of the cell wall that occurs when multiple synergistic enzymes are present in a cell. For example, combining an endoglucanase with an apoplast promoter, a hemicellulase with a vacuole promoter, and an exoglucanase with a chloroplast promoter, sequesters each enzyme in a different part of the cell and achieves the advantages listed above. This method circumvents the limit on enzyme mass that can be expressed in a single organelle or location of the cell.

The localization of a nuclear-encoded protein (e.g., enzyme polypeptide) within the cell is known to be determined by the amino acid sequence of the protein. The protein localization can be altered by modifying the nucleotide sequence that encodes the protein in such a manner as to alter the protein's amino acid sequence. The polynucleotide sequences encoding ligno-cellulolytic enzymes can be altered to redirect the cellular localization of the encoded enzymes by any suitable method (see, e.g., Dai et al., Trans. Res., 2005, 14: 627, the entire contents of which are herein incorporated by reference). In some embodiments of the invention, protein localization is altered by fusing a sequence encoding a signal peptide to the sequence encoding the enzyme polypeptide. Signal peptides that may be used in accordance with the invention include a secretion signal from sea anemone equistatin (which allows localization to apoplasts) and secretion signals comprising the KDEL motif (which allows localization to endoplasmic reticulum).

### Expression Vectors

Nucleic acid constructs according to the present invention may be cloned into a vector, such as, for example, a plasmid. Vectors suitable for transforming plant cells include, but are not limited to, Ti plasmids from Agrobacterium tumefaciens (J. Darnell, H. F. Lodish and D. Baltimore, “Molecular Cell Biology”, 2^{nd} Ed., 1990, Scientific American Books: New York), a plasmid containing a β-glucuronidase gene and a cauliflower mosaic virus (CaMV) promoter plus a leader sequence from alfalfa mosaic virus (J. C. Sanford et al., Plant Mol. Biol. 1993, 22: 751-765) or a plasmid containing a bar gene cloned downstream from a CaMV 35S promoter and a tobacco mosaic virus (TMV) leader. Other plasmids may additionally contain introns, such as that derived from alcohol dehydrogenase (Adh1), or other DNA sequences. The size of the vector is not a limiting factor.

For constructs intended to be used in Agrobacterium-mediated transformation, the plasmid may contain an origin of replication that allows it to replicate in Agrobacterium and a high copy number origin of replication functional in E. coli. This permits facile production and testing of transgenes in E. coli prior to transfer to Agrobacterium for subsequent introduction in plants. Resistance genes can be carried on the vector, one for selection in bacteria, for example, streptomycin, and another that will function in plants, for example, a gene encoding kanamycin resistance or herbicide resistance. Also present on the vector are restriction endonuclease sites for the addition of one or more transgenes and directional T-DNA border sequences which, when recognized by the transfer functions of Agrobacterium, delimit the DNA region that will be transferred to the plant.

Methods of preparation of nucleic acid constructs and expression vectors are well known in the art and can be found described in several textbooks such as, for example, J. Sambrook, E. F. Fritsch and T. Maniatis, “Molecular Cloning: A Laboratory Manual”, 1989, Cold Spring Harbor Laboratory: Cold Spring Harbor, and T. J. Silhavy, M. L. Berman, and L. W. Enquist, “Experiments with Gene Fusions”, 1984, Cold Spring Harbor Laboratory: Cold Spring Harbor; F. M. Ausubel et al., “Current Protocols in Molecular Biology”, 1989, John Wiley & Sons: New York.

Additional desirable properties of the transgenic plants may include, but are not limited to, ability to adapt for growth in various climates and soil conditions; well studied genetic model system; incorporation of bioconfinement features such as male (or total) sterile flowers; incorporation of phytoremediation features such as contaminant hyperaccumulation, greater biomass, or promotion of contaminant-degrading mycorrhizae.

### III. Preparation of Transgenic Plants

Nucleic acid constructs, such as those described above, can be used to transform any plant including monocots and dicots. In some embodiments, plants are green field plants. In other embodiments, plants are grown specifically for “biomass energy” and/or phytoremediation. Examples of suitable plants for use in the methods of the present invention include, but are not limited to, corn, switchgrass, sorghum, miscanthus, sugarcane, poplar, pine, wheat, rice, soy, cotton, barley, turf grass, tobacco, bamboo, rape, sugar beet, sunflower, willow, and eucalyptus. Using transformation methods, genetically modified plants, plant cells, plant tissue, seeds, and the like can be obtained.

Transformation according to the present invention may be performed by any suitable method. In certain embodiments, transformation comprises steps of introducing a nucleic acid construct, as described above, into a plant cell or protoplast to obtain a stably transformed plant cell or protoplast; and regenerating a whole plant from the stably transformed plant cell or protoplast.

### Cell Transformation

Delivery or introduction of a nucleic acid construct into eukaryotic cells may be accomplished using any of a variety of methods. The method used for the transformation is not critical to the instant invention. Suitable techniques include, but are not limited to, non-biological methods, such as microinjection, microprojectile bombardment, electroporation, induced uptake, and aerosol beam injection, as well as biological methods such as direct DNA uptake, liposomes and Agrobacterium-mediated transformation. Any combinations of the above methods that provide for efficient transformation of plant cells or protoplasts may also be used in the practice of the invention.

Methods of introduction of nucleic acid constructs into plant cells or protoplasts have been described. See, for example, “Methods for Plant Molecular Biology”, Weissbach and Weissbach (Eds.), 1989, Academic Press, Inc; “Plant Cell, Tissue and Organ Culture: Fundamental Methods”, 1995, Springer-Verlag: Berlin, Germany; and U.S. Pat. Nos. 4,945,050; 5,036,006; 5,100,792; 5,240,855; 5,302,523; 5,322,783; 5,324,646; 5,384,253; 5,464,765; 5,538,877; 5,538,880; 5,550,318; 5,563,055; and 5,591,616).

In particular, electroporation has frequently been used to transform plant cells (see, for example, U.S. Pat. No. 5,384,253). This method is generally performed using friable tissues (such as a suspension culture of cells or embryogenic callus) or target recipient cells from immature embryos or other organized tissue that have been rendered more susceptible to transformation by electroporation by exposing them to pectin-degrading enzymes or by mechanically wounding them in a controlled manner. Intact cells of maize (see, for example, K. D'Halluin et al., Plant cell, 1992, 4: 1495-1505; C. A. Rhodes et al., Methods Mol. Biol. 1995, 55: 121-131; and U.S. Pat. No. 5,384,253), wheat, tomato, soybean, and tobacco have been transformed by electroporation. As reviewed, for example, by G. W. Bates (Methods Mol. Biol. 1999, 111: 359-366), electroporation can also be used to transform protoplasts.

Another method of transformation is microprojectile bombardment (see, for example, U.S. Pat. Nos. 5,538,880; 5,550,318; and 5,610,042; and WO 94/09699). In this method, nucleic acids are delivered to living cells by coating or precipitating the nucleic acids onto a particle or microprojectile (for example tungsten, platinum or gold), and propelling the coated microprojectile into the living cell. Microprojectile bombardment techniques are widely applicable, and may be used to transform virtually any monocotyledonous or dicotyledonous plant species (see, for example, U.S. Pat. Nos. 5,036,006; 5,302,523; 5,322,783 and 5,563,055; WO 95/06128; A. Ritala et al., Plant Mol. Biol. 1994, 24: 317-325; L. A. Hengens et al., Plant Mol. Biol. 1993, 23: 643-669; L. A. Hengens et al., Plant Mol. Biol. 1993, 22: 1101-1127; C. M. Buising and R. M. Benbow, Mol. Gen. Genet. 1994, 243: 71-81; C. Singsit et al., Transgenic Res. 1997, 6: 169-176).

The use of Agrobacterium-mediated transformation of plant cells is well known in the art (see, for example, U.S. Pat. No. 5,563,055). This method has long been used in the transformation of dicotyledonous plants, including Arabidopsis and tobacco, and has recently also become applicable to monocotyledonous plants, such as rice, wheat, barley and maize (see, for example, U.S. Pat. No. 5,591,616). In plant strains where Agrobacterium-mediated transformation is efficient, it is often the method of choice because of the facile and defined nature of the gene transfer. Agrobacterium-mediated transformation of plant cells is carried out in two phases. First, the steps of cloning and DNA modifications are performed in E. coli, and then the plasmid containing the gene construct of interest is transferred by heat shock treatment into Agrobacterium, and the resulting Agrobacterium strain is used to transform plant cells.

Transformation of plant protoplasts can be achieved using methods based on calcium phosphate precipitation, polyethylene glycol treatment, electroporation, and combinations of these treatments (see, e.g., I. Potrykus et al., Mol. Gen. Genet. 1985, 199: 169-177; M. E. Fromm et al., Nature, 1986, 31: 791-793; J. Callis et al., Genes Dev. 1987, 1: 1183-1200; S. Omirulleh et al., Plant Mol. Biol. 1993, 21: 415-428).

Alternative methods of plant cell transformation, which have been reviewed, for example, by M. Rakoczy-Trojanowska (Cell Mol. Biol. Lett. 2002, 7: 849-858), can also be used in the practice of the present invention.

The successful delivery of the nucleic acid construct into the host plant cell or protoplast may be preliminarily evaluated visually. Selection of stably transformed plant cells can be performed, for example, by introducing into the cell, a nucleic acid construct comprising a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Examples of herbicides which may be used include phosphinothricin and glyphosate. Potentially transformed cells then are exposed to the selective agent. Cells where the resistance-conferring gene has been integrated and expressed at sufficient levels to permit cell survival will generally be present in the population of surviving cells.

Alternatively, host cells comprising a nucleic acid sequence of the invention and which express its gene product may be identified and selected by a variety of procedures, including, DNA-DNA or DNA-RNA hybridization and protein bioassay or immunoassay techniques such as membrane, solution or chip-based technologies for the detection and/or quantification of nucleic acid or protein.

Plant cells are available from a wide range of sources including the American Type Culture Collection (Rockland, MD), or from any of a number of seed companies including, for example, A. Atlee Burpee Seed Co. (Warminster, Pa.), Park Seed Co. (Greenwood, S.C.), Johnny Seed Co. (Albion, Me.), or Northrup King Seeds (Hartsville, S.C.). Descriptions and sources of useful host cells are also found in I. K. Vasil, “Cell Culture and Somatic Cell Genetics of Plants”, Vol. I, II and II; 1984, Laboratory Procedures and Their Applications Academic Press: New York; R. A. Dixon et al., “Plant Cell Culture-A Practical Approach”, 1985, IRL Press: Oxford University; and Green et al., “Plant Tissue and Cell Culture”, 1987, Academic Press: New York.

Plant cells or protoplasts stably transformed according to the present invention are provided herein.

### Plant Regeneration

In plants, every cell is capable of regenerating into a mature plant, and in addition contributing to the germ line such that subsequent generations of the plant will contain the transgene of interest. Stably transformed cells may be grown into plants according to conventional ways (see, for example, McCormick et al., Plant Cell Reports, 1986, 5: 81-84). Plant regeneration from cultured protoplasts has been described, for example by Evans et al., “Handbook of Plant Cell Cultures”, Vol. 1, 1983, MacMilan Publishing Co: New York; and I. R. Vasil (Ed.), “Cell Culture and Somatic Cell Genetics of Plants”, Vol. I (1984) and Vol. II (1986), Acad. Press: Orlando.

Means for regeneration vary from species to species of plants, but generally a suspension of transformed protoplasts or a Petri plate containing transformed explants is first provided. Callus tissue is formed and shoots may be induced from callus and subsequently roots. Alternatively, somatic embryo formation can be induced in the callus tissue. These somatic embryos germinate as natural embryos to form plants. The culture media will generally contain various amino acids and plant hormones, such as auxin and cytokinins Glutamic acid and proline may also be added to the medium. Efficient regeneration generally depends on the medium, on the genotype, and on the history of the culture.

Regeneration from transformed individual cells to obtain transgenic whole plants has been shown to be possible for a large number of plants. For example, regeneration has been demonstrated for dicots (such as apple; Malus pumila; blackberry, Rubus; Blackberry/raspberry hybrid, Rubus; red raspberry, Rubus; carrot; Daucus carona; cauliflower; Brassica oleracea; celery; Apium graveolens; cucumber; Cucumis sativus; eggplant; Solanum melongena; lettuce; Lactuca sativa; potato; Solanum tuberosum; rape; Brassica napus; soybean (wild); Glycine canescens; strawberry; Fragaria x ananassa; tomato; Lycopersicon esculentum; walnut; Juglans regia; melon; Cucumis melo; grape; Vitis vinifera; and mango; Mangifera indica) as well as for monocots (such as rice; Oryza sativa; rye, Secale cereale; and Maize).

Primary transgenic plants may then be grown using conventional methods. Various techniques for plant cultivation are well known in the art. Plants can be grown in soil, or alternatively can be grown hydroponically (see, for example, U.S. Pat. Nos. 5,364,451; 5,393,426; and 5,785,735). Primary transgenic plants may be either pollinated with the same transformed strain or with a different strain and the resulting hybrid having the desired phenotypic characteristics identified and selected. Two or more generations may be grown to ensure that the subject phenotypic characteristics is stably maintained and inherited and then seeds are harvested to ensure that the desired phenotype or other property has been achieved.

As is well known in the art, plants may be grown in different media such as soil, growth solution or water.

Selection of plants that have been transformed with the construct may be performed by any suitable method, for example, with northern blot, Southern blot, herbicide resistance screening, antibiotic resistance screening or any combinations of these or other methods. The Southern blot and northern blot techniques, which test for the presence (in a plant tissue) of a nucleic acid sequence of interest and of its corresponding RNA, respectively, are standard methods (see, for example, Sambrook & Russell, “Molecular Cloning”, 2001, Cold Spring Harbor Laboratory Press: Cold Spring Harbor).

### IV. Uses of Inventive Transgenic Plants

The transgenic plants and plant parts disclosed herein may be used advantageously in a variety of applications. More specifically, the present invention, which involves genetically engineering plants for both increased biomass and expression of lignocellulolytic enzyme polypeptides, results in downstream process innovations and/or improvements in a variety of applications including ethanol production, phytoremediation and hydrogen production.

### A—Ethanol Production

Plants transformed according to the present invention provide a means of increasing ethanol yields, reducing pretreatment costs by reducing acid/heat pretreatment requirements for saccharification of biomass; and/or reducing other plant production and processing costs, such as by allowing multi-applications and isolation of commercially valuable by-products.

Plant Culture. As already mentioned above, farmers can grow different transgenic plants of the present invention (e.g., different variety of transgenic corn, each expressing a lignocellulolytic enzyme polypeptide or a combination of enzyme polypeptides) simultaneously, achieving the desired “blend” of enzyme polypeptides produced by changing the seed ratio.

Plant Harvest. Transgenic plants of the present invention can be harvested as known in the art. For example, current techniques may cut corn stover at the same time as the grain is harvested, but leave the stover lying in the field for later collection. However, dirt collected by the stover can interfere with ethanol production from lignocellulosic material. The present invention provides a method in which transgenic plants are cut, collected, stored, and transported so as to minimize soil contact. In addition to minimizing interference from dirt with ethanol production, this method can result in reduction in harvest and transportation costs.

### Tempering

Inventive methods include a tempering phase that conditions the biomass for pretreatment and hydrolysis.

Tempering may facilitate reducing severity of pretreatment conditions to achieve a desired glucan conversion yield and/or improving hydrolysis and glucan conversion after treatment. For example, a typical yield from biomass that has been pretreated under standard pretreatment conditions (e.g., 1% sulfuric acid, 170° C., for 10 minutes) is at least 80% glucan conversion. When tempered as described herein, the same typical yield may be achieved under less severe pretreatment conditions and/or with reduced amounts of externally applied enzymes. Less severe pretreatment conditions may comprise, for example, reduced acid concentrations, lower incubation temperatures, and/or shorter pretreatment times.

In some embodiments, when tempered as described herein and using the same pretreatment conditions, typical yield may be increased above at least 80% glucan conversion.

Without wishing to be bound by any particular theory, tempering may facilitate such improvements by, for example, allowing activation of endoplant enzyme polypeptides after harvest, increasing susceptibility of lignin and hemicellulose to traditional pretreatment, and/or increasing accessibility of polysaccharides (e.g., cellulose).

A variety of techniques for tempering may be used. In some embodiments, tempering comprises increasing the temperature of the biomass to activate thermophilic enzymes. Increasing the temperature to activate thermophilic enzymes may be achieved, for example, by one or more of ensilement, grinding, pelleting, and warm water suspension/slurries. In some embodiments, tempering comprises disrupting cell walls. Cell wall disruption may be achieved, for example, by sonication and/or liquid extraction to release enzyme polypeptides from sequestered locations in the plant (which may allow further activation and/or extraction to be added back after pretreatment). In some embodiments, tempering comprises adding accessory enzyme polypeptides during an incubation period before pretreatment. Such accessory enzyme polypeptides may weaken cross linking and improve accessibilty of the biomass to embedded glucanases or xylanases. In some embodiments, tempering comprises incubating the biomass in a particular set of conditions (e.g., a particular temperature, particular pH, and/or particular moisture conditions). Such incubations may in some embodiments increase susceptibility to various glucanases and/or accessory enzyme polypeptides present in the plant tissues or added to the sample. For example, samples may be tempered as a liquid slurry (e.g., comprising about 10% to about 30% total solids) under conditions favorable to activate lignocellulolytic enzymes, such as occurs in “acceleration” protocols described in the Examples. In some embodimetns, samples are tempered as a liquid slurry for about 1 to about 48 hours. In some embodiments, conditions favorable to activate lignocellulolytic enzymes comprise a pH of about 4 to about 7 and a temperature of about 25° C. to about 100° C. Alternatively or additionally, samples may be tempered as a lower moisture ensilement (e.g., about 40% to about 60% total solids) under anaerobic conditions. In some embodiments, samples are ensiled for about 21 days to several months.

In some embodiments, tempering is integrated with other processes such as one or more of harvest, storage, and transportation of biomass. For example, biomass can be ensiled under conditions that condition the biomass for subsequent pretreatment and hydrolysis; that is, storage and tempering are combined. In some embodiments, during ensilement of biomass, temperatures are increased in the ensiled material such that thermally active embedded enzymes are activated. Ensilement conditions may allow preservation of biomass while providing sufficient time for enzyme polypeptides to affect characteristics of the biomass (such as, for example, amenability to pretreatment and improvement of subsequent hydrolysis).

In some embodiments, samples are tempered both by ensilement and by acceleration.

In some embodiments, the tempering phase precedes entirely the pretreatment phase. In some embodiments, the tempering phase overlaps with the pretreatment phase.

In some embodiments as described herein, transgenic plants express more than one lignocellulolytic enzyme polypeptide. In some such embodiments, it may be desirable to activate enzyme polypeptides sequentially. It may be desirable to do so, for example, if the efficiency of endoplant enzymes is a function of the sequence in which they are activated. For example, beta-glucosidases may be most efficient after endo- and exoglucanases have cleaved cellulose into dimers, and cellulases and hemicellulases may be more efficient when accessory enzymes have reduced cross-linkages between cellulose, hemicellulose, and lignin. Accordingly, in some embodiments, cellulases might be activated after ferulic acid esterases (FAEs) have had the opportunity to cleave ferulate-polysaccharide-lignin complexes, or after other accessory enzymes have had the opportunity to cleave cellulose-hemicellulose cross linkages.

Sequential activation could be attained, for example, by using enzymes with different peak temperature and/or pH optima. Increasing temperature continually or stepwise (e.g., during a tempering step), could thereby allow activation of enzyme polypeptides with lower temperature optima first. For example, a wound-induced promoter could be used to produce a non-thermostable enzyme polypeptide after harvesting that breaks lingin cross-links and leads to cell death, before increasing temperature during tempering to activate a thermostable cellulase in the biomass.

In some embodiments as described herein, lignocellulosic enzyme polypeptides are specifically targeted to organelles and/or plant parts. In some embodiments, lignocellulosic enzyme polypeptides are specifically targeted to seeds. Cell wall hydrolyzing enzymes in the grain could improve yields of fermentable sugars by targeting the cellulose and hemicelluolose in the grain bran and fiber, or could loosen or weaken the outer layers of the grain kernel, making it easier to mill. Starch in corn grain is often processed to produce ethanol, but significant quantitiues of cellulose and hemicellulose from the bran and fiber are not used. In some embodiments, incorporating a tempering step prior to starch hydrolysis (e.g., of transgenic corn grain), endogenous enzymes can act on the fiber and bran and increase the yield of fermentable sugars. In some embodiments, dry seed (e.g., dry wheat) is tempered by soaking in water at a slightly elevated temperature for several hours before further processing. Such a tempering step may decrease the energy required for milling and increase the quality and eventual yield. Endogenous enzymes in the grain may also provide additional benefits.

In some embodiments, tempering comprises externally applying an amount of at least one lignocellulolytic enzyme polypeptide. External application of lignocellulolytic enzyme polypeptides is discussed in more detail in the “Saccharification” section.

In some embodiments, the seed or grain of a transgenic plant is tempered.

### Pretreatment

Conventional methods include physical, chemical, and/or biological pretreatments. For example, physical pretreatment techniques can include one or more of various types of milling, crushing, irradiation, steaming/steam explosion, and hydrothermolysis. Chemical pretreatment techniques can include acid, alkaline, organic solvent, ammonia, sulfur dioxide, carbon dioxide, and pH-controlled hydrothermolysis. Biological pretreatment techniques can involve applying lignin-solubilizing microorganisms (T.-A. Hsu, “Handbook on Bioethanol: Production and Utilization”, C. E. Wyman (Ed.), 1996, Taylor & Francis: Washington, D.C., 179-212; P. Ghosh and A. Singh, A., Adv. Appl. Microbiol., 1993, 39: 295-333; J. D. McMillan, in “Enzymatic Conversion of Biomass for Fuels Production”, M. Himmel et al., (Eds.), 1994, Chapter 15, ACS Symposium Series 566, American Chemical Society: B. Hahn-Hagerdal, Enz. Microb. Tech., 1996, 18: 312-331; and L. Vallander and K. E. L. Eriksson, Adv. Biochem. Eng./Biotechnol., 1990, 42: 63-95). The purpose of the pretreatment step is to break down the lignin and carbohydrate structure to make the cellulose fraction accessible to cellulolytic enzymes.

Simultaneous use of transgenic plants that express one or more cellulases, one or more hemicellulases and/or one or more ligninases according to the present invention reduces or eliminates expensive grinding of the biomass, reduces or eliminates the need for heat and strong acid required to strip lignin and hemicellulose away from cellulose before hydrolyzing the cellulose.

In some embodiments, lignocellulosic biomass of plant parts obtained from inventive transgenic plants is more easily hydrolyzable than that of non-transgenic plants. Thus, the extent and/or severity of pretreatment required to achieve a particular level of hydrolysis is reduced. Therefore, the present invention in some embodiments provides improvements over existing pretreatment methods. Such improvements may include one or more of: reduction of biomass grinding, elimination of biomass grinding, reduction of the pretreatment temperature, elimination of heat in the pretreatment, reduction of the strength of acid in the pretreatment step, elimination of acid in the pretreatment step, and any combination thereof.

In some embodiments, lower temperatures of pretreatment may be used to achieve a desired level of hydrolysis. In some embodiments, pretreating is performed at temperatures below about 175° C., below about 145° C., or below about 115° C. For example, under some conditions, the yield of hydrolysis products from lignocellulosic biomass from transgenic plant parts pretreated at about 140° C. is comparable to the yield of hydrolysis products from non-transgenic plant parts pretreated at about 170° C. Under some conditions, the yield of hydrolysis products from lignocellulosic biomass from transgenic plant parts pretreated at about 170° C. is above about 60%, above about 70%, above about 80%, or above about 90% of theoretical yields. Under some conditions, the yield of hydrolysis products from lignocellulosic biomass from transgenic plant parts pretreated at about 140° C. is above about 60%, above about 70%, or above about 80% of theoretical yields. Under some conditions, the yield of hydrolysis products from lignocellulosic biomass from transgenic plant parts pretreated at about 110° C. is above about 40%, above about 50%, or above about 60% of theoretical yields. Such yields from transgenic plant parts can represent an increase of up to about 20% of yields from non-transgenic plant parts.

In some embodiments, such improvements are observed in inventive transgenic plants expressing a lignocellulolytic enzyme polypeptide at a level less than about 0.5%, less than about 0.4%, less than about 0.3%, less than about 0.2%, or less than about 0.1% of total soluble protein. Without wishing to be bound by any particular theory, the inventors propose that low levels of enzyme expression may facilitate modifying the cell wall, possibly by nicking cellulose or hemicellulose strands. Such modification of the cell wall may make the biomass more susceptible to pretreatment. Thus, biomass from inventive transgenic plants expressing low levels of lignocellulolytic enzymes may require less pretreatment, and/or pretreatment in less severe conditions.

In certain embodiments, the pretreated material is used for saccharification without further manipulation. In other embodiments, it may be desired to process the plant tissue so as to produce an extract comprising the lignocellulolytic enzyme polypeptide(s). In this case, the extraction is carried out in the presence of components known in the art to favor extraction of active enzymes from plant tissue and/or to enhance the degradation of cell-wall polysaccharides in the lignocellulosic biomass. Such components include, but are not limited to, salts, chelators, detergents, antioxidants, polyvinylpyrrolidone (PVP), and polyvinylpolypyrrolidone (PVPP). The remaining plant tissue may then be submitted to a pretreatment process.

### Saccharification

In saccharification (or enzymatic hydrolysis), lignocellulose is converted into fermentable sugars (i.e. glucose monomers) by lignocellulolytic enzyme polypeptides present in the pretreated material. If desired, external cellulolytic enzyme polypeptides (i.e., enzymes not produced by the transgenic plants being processed) may be added to this mixture. Extracts comprising lignocellulolytic enzyme polypeptides obtained as described above can be added back to the lignocellulosic biomass before saccharification. Here again, external cellulolytic enzyme polypeptides may be added to the saccharification reaction mixture.

In some embodiments, the amount of externally applied enzyme polypeptide that is required to achieve a particular level of hydrolysis of lignocellulosic biomass from inventive transgenic plants is reduced as compared to the amount required to achieve a similar level of hydrolysis of lignocellulosic biomass from non-transgenic plants. For example, in some embodiments, processing transgenic lignocellulosic biomass in the presence of as low as 15 mg externally applied cellulase per gram of biomass (15 mg/g) yields a similar level of hydrolysis as processing non-transgenic lignocellulosic biomass in the presence of 100 mg/g cellulase. This represents a reduction of almost 90% of cellulases needed for hydrolysis can be achieved when processing biomass from inventive transgenic plants. Such a reduction in externally applied cellulases used can represent significant cost savings.

In some embodiments, a mixture of enzyme polypeptides each having different enzyme activities (e.g., exoglucanase, endoglucanase, hemi-cellulase, beta-glucosidase, and combinations thereof), and/or an enzyme polypeptide having more than one enzyme activity (e.g., exoglucanase, endoglucanase, hemi-cellulase, beta-glucosidase, and combinations thereof) is added during a “treatment” step to promote saccharification. Without wishing to be bound by any particular theory, such combinations of enzyme activity, whether through the activity of an enzyme complex or other mixture of enzymes, may allow a greater degree of hydrolysis than can be achieved with a single enzyme activity alone. Commercially available enzyme complexes that can be employed in the practice of the invention include, but are not limited to, ACCELLERASE™ 1000 (Genencor), which contains multiple enzyme activities, mainly exoglucanase, endoglucanase, hemi-cellulase, and beta-glucosidase.

In some embodiments, a crude extract comprising one or more lignocellulosic enzyme polypeptides is added. Such crude extracts may be obtained, for example, from transgenic plant parts such as those provided by the present invention. In some embodiments, the crude extracts comprise lignocellulosic enzyme polypeptides that are encoded by recombinant polynucleotides. In some embodiments, the crude extracts comrpise lignocellulosic enzyme polypeptides that are fused to affinity tags.

Saccharification is generally performed in stirred-tank reactors or fermentors under controlled pH, temperature, and mixing conditions. A saccharification step may last up to 200 hours. Saccharification may be carried out at temperatures from about 30° C. to about 65° C., in particular around 50° C., and at a pH in the range of between about 4 and about 5, in particular, around pH 4.5. Saccharification can be performed on the whole pretreated material.

The present Applicants have shown that adding cellulases to E1-transformed plants increases total glucose production compared to adding cellulases to non-transgenic plants, which suggests that simply using transgenic E1 plants with current external cellulase techniques can substantially increase ethanol yields. The experiment also indicates that adding cellulases to E1 plants increases total glucose production compared to adding cellulases to non-transgenic plants. This is an important result since it suggests that simply using transgenic E1 plants with current external cellulase techniques can substantially increase ethanol yields in the presence or absence of pretreatment processes.

Fermentation. In the fermentation step, sugars, released from the lignocellulose as a result of the pretreatment and enzymatic hydrolysis steps, are fermented to one or more organic substances, e.g., ethanol, by a fermenting microorganism, such as yeasts and/or bacteria. The fermentation can also be carried out simultaneously with the enzymatic hydrolysis in the same vessels, again under controlled pH, temperature and mixing conditions. When saccharification and fermentation are performed simultaneously in the same vessel, the process is generally termed simultaneous saccharification and fermentation or SSF.

Fermenting microorganisms and methods for their use in ethanol production are known in the art (Sheehan, “The Road to Bioethanol: A Strategic Perspective of the US Department of Energy's National Ethanol Program” In: “Glycosyl Hydrolases For Biomass Conversion”, ACS Symposium Series 769, 2001, American Chemical Society: Washington, D.C.). Existing ethanol production methods that utilize corn grain as the biomass typically involve the use of yeast, particularly strains of Saccharomyces cerevisiae. Such strains can be utilized in the methods of the invention. While such strains may be preferred for the production of ethanol from glucose that is derived from the degradation of cellulose and/or starch, the methods of the present invention do not depend on the use of a particular microorganism, or of a strain thereof, or of any particular combination of said microorganisms and said strains.

Yeast or other microorganisms are typically added to the hydrolysate and the fermentation is allowed to proceed for 24-96 hours, such as 35-60 hours. The temperature of fermentation is typically between 26-40° C., such as 32° C., and at a pH between 3 and 6, such as about pH 4-5.

A fermentation stimulator may be used to further improve the fermentation process, in particular, the performance of the fermenting microorganism, such as, rate enhancement and ethanol yield. Fermentation stimulators for growth include vitamins and minerals. Examples of vitamins include multivitamin, biotin, pantothenate, nicotinic acid, meso-inositol, thiamine, pyridoxine, para-aminobenzoic acid, folic acid, riboflavin, and vitamins A, B, C, D, and E (Alfenore et al., “Improving ethanol production and viability of Saccharomyces cerevisiae by a vitamin feeding strategy during fed-batch process”, 2002, Springer-Verlag). Examples of minerals include minerals and mineral salts that can supply nutrients comprising phosphate, potassium, manganese, sulfur, calcium, iron, zinc, magnesium and copper.

Recovery. Following fermentation (or SSF), the mash is distilled to extract the ethanol. Ethanol with a purity greater than 96 vol. % can be obtained.

By-Products. The hydrolysis process of lignocellulosic raw material also releases by-products such as weak acids, furans, and phenolic compounds, which are inhibitory to the fermentation process. Removing such by-products may enhance fermentation. In particular, lignin and lignin breakdown products such as phenols, produced by enzymatic activity and by other processing activities, from the saccharified cellulosic biomass is likely to be important to speeding up fermentation and maintaining optimum viscosity.

Thus, in another aspect, the present invention provides methods of speeding up fermentation which comprise removing, from the hydrolysate, products of the enzymatic process that cannot be fermented. Such products comprise, but are not limited to, lignin, lignin breakdown products, phenols, and furans. In certain embodiments, products of the enzymatic process that cannot be fermented can be separated and used subsequently. For example, the products can be burned to provide heat required in some steps of the ethanol production such as saccharification, fermentation, and ethanol distillation, thereby reducing costs by reducing the need for current external energy sources such as natural gas. Alternatively, such by-products may have commercial value. For example, phenols can find applications as chemical intermediates for a wide variety of applications, ranging from plastics to pharmaceuticals and agricultural chemicals. Phenol condensed to with aldehydes (e.g., methanol) make resinous compounds, which are the basis of plastics which are used in electrical equipment and as bonding agents in manufacturing wood products such as plywood and medium density fiberboard (MDF).

Separation of by-products from the hydrolysate can be done using a variety of chemical and physical techniques that rely on the different chemical and physical properties of the by-products (e.g., lignin and phenols). Such techniques include, but are not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, distillation, or extraction.

Some of the hydrolysis by-products, such as phenols, or fermentation/processing products, such as methanol, can be used as ethanol denaturants. Currently about 5% gasoline is added immediately to distilled ethanol as a denaturant under the Bureau of Alcohol, Tobacco and Firearms regulations, to prevent unauthorized non-fuel use. This requires shipping gasoline to the ethanol production plant, then shipping the gas back with the ethanol to the refinery. The gas also impedes the use of ethanol-optimized engines that make use of ethanol's higher compression ratio and higher octane to improve performance. Using transgenic plant derived phenols and/or methanol as denaturants in lieu of gasoline can reduce costs and increase automotive engine design alternatives.

Reducing Lignin Content. Another way of reducing lignin and lignin breakdown products that are not fermentable in hydrolysate is to reduce lignin content in transgenic plant of the present invention. Such methods have been developed and can be used to modify the inventive plants (see, for example, U.S. Pat. Nos. 6,441,272 and 6,969,784, U.S. Pat. Appln. No. 2003-0172395, US and PCT publication No. WO 00/71670).

Combined Starch Hydrolysis and Cellulolytic Material Hydrolysis. The transgenic plants and plant parts disclosed herein can be used in methods involving combined hydrolysis of starch and of cellulosic material for increased ethanol yields. In addition to providing enhanced yields of ethanol, these methods can be performed in existing starch-based ethanol processing facilities.

Starch is a glucose polymer that is easily hydrolyzed to individual glucose molecules for fermentation. Starch hydrolysis may be performed in the presence of an amylolytic microorganism or enzymes such as amylase enzymes. In certain embodiments of the invention, starch hydrolysis is performed in the presence of at least one amylase enzyme. Examples of suitable amylase enzymes include α-amylase (which randomly cleaves the α(1-4)glycosidic linkages of amylose to yield dextrin, maltose or glucose molecules) and glucoamylase (which cleaves the α(1-4) and α(1-6)glycosidic linkages of amylose and amylopectin to yield glucose).

In the inventive methods, hydrolysis of starch and hydrolysis of cellulosic material can be performed simultaneously (i.e., at the same time) under identical conditions (e.g., under conditions commonly used for starch hydrolysis). Alternatively, the hydrolytic reactions can be performed sequentially (e.g., hydrolysis of lignocellulose can be performed prior to hydrolysis of starch). When starch and cellulosic material are hydrolyzed simultaneously, the conditions are preferably selected to promote starch degradation and to activate lignocellulolytic enzyme polypeptide(s) for the degradation of lignocellulose. Factors that can be varied to optimize such conditions include physical processing of the plants or plant parts, and reaction conditions such as pH, temperature, viscosity, processing times, and addition of amylase enzymes for starch hydrolysis.

The inventive methods may use transgenic plants (or plant parts) alone or a mixture of non-transgenic plants (or plant parts) and plants (or plant parts) transformed according to the present invention. Suitable plants include any plants that can be employed in starch-based ethanol production (e.g., corn, wheat, potato, cassava, etc). For example, the present inventive methods may be used to increase ethanol yields from corn grains.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1

### Generation of Transgenic Tobacco

To generate the transgenic tobacco, wild-type tobacco was transformed with the E1 and then AtFLC genes using Agrobacterium tumefaciens as described below.

Description of the Donor. The endo-1,4-β-glucanase E1 gene (GenBank Accession No. U33212) was isolated from the thermophilic bacterium Acidothermus cellulolyticus. This bacterium was originally isolated from decaying wood in an acidic, thermal pool at Yellowstone National Park and deposited with the American Type Culture Collection (ATCC, Manassas, Va.) under collection number 43068 (A. Mohagheghi et al., Int. J. System. Baceril., 1986, 36: 435-443; Tucker et al., Biotechnology, 1989, 7: 817-820). As already mentioned herein, the bacterium has been characterized with the ability to hydrolyze and degrade plant cellulose.

For transformation into tobacco, the E1 catalytic domain was isolated from the genomic sequence and contained by 950-2020 listed in Accession No. U33212. To generate the E1-catalytic construct, a stop codon was introduced after the codon specifying Val-358 of E1 through Polymerase Chain Reaction (PCR), and the 5′ end of the gene was fused to the 21 amino acids in the amino-terminal soybean vegetative storage protein VSPβ (GenBank Accession No. M76980) (Ziegelhoffer et al., Mol. Breed, 2001, 8: 147-158) in order to target the protein to the apoplast. For cloning purposes, a Sad site was added to the 3′ end of the E1 gene following the stop codon and an XbaI site at the 5′end of the VSPI3 sequence.

The inhibitor Flowering Locus C gene, or FLC (accession # BK000546) is a dosage-dependent repressor of flowering in Arabidopsis (S. D. Michaels and R. M. Amasino, Plant Cell, 1999, 11: 949-956), which operates by negatively regulating the expression of genes that promote flowering, such as SOC1 and FT. The 591 by cDNA was isolated from Arabidopsis and used without modification for transformation into tobacco.

Description of the Recipient. The recipient organism was Nicotiana tabacum W38, a commonly used variety for laboratory studies. Tobacco is a very well characterized crop that has been cultivated for centuries.

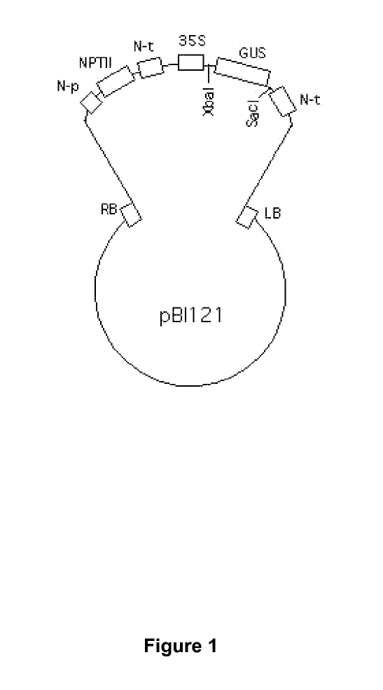

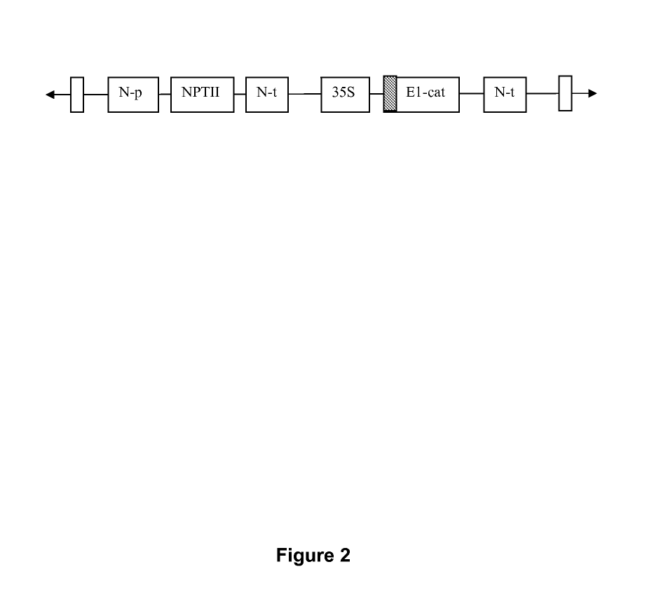

Description of the Vector and the Transformation Process. The E1 transformation vector was constructed from an existing pBI121 binary Ti vector used for agrobacteria mediated transformation (Jefferson et al., EMBO J., 1987, 6: 3901). Through standard agrobacteria transformation, DNA sequences in between the right and left borders are stably transferred into the plant genome. The complete sequence of pBI121 is 14,758 by (GenBank Accession No. AF502128) and contains resistance to the antibiotic kanamycin and the GUS gene in between its right and left border sequence (as presented on FIG. 1). For the development of pBI121-E1, the β-glucuronidase gene was excised through digestion with XbaI and SacI and replaced with the VSPβ/E1 construct (as shown on FIG. 2).

Tobacco leaf explants were transformed with pBI121-E1 according to standard procedures (Horsch et al., Science, 1985, 227: 1229). Leaf explants were taken from the second and third fully expanded leaves of 3-week old in vitro shoot cultures of Nicotiana tabacum W38 maintained on MS medium. After pre-culture, explants were dropped into a suspension of Agrobacterium cells containing the modified pBI121 vector obtained from an overnight culture. Leaf pieces were selected on 100 mg/L kanamycin and plantlets (typically 2 or 3) developed 10-14 days later from callus formed along cut leaf edges. Plantlets were excised and rooted on MS media containing 100 mg/L kanamycin in Magenta GA7 boxes (Ziegelhoffer et al., Mol. Breed, 2001, 8: 147-158).

Transformed plants were confirmed through genomic PCR amplification of the E1 gene in parallel with measuring the hydrolysis of cellulose using leaf extracts (Ziegelhoffer et al., Mol. Breed, 2001, 8: 147-158). Plants positive for both the presence of the E1 gene and E1 activity were grown to maturity and seeds were collected for further work. Stable expression of the E1 gene and E1 activity were observed for multiple generations of tobacco after the transformation event.

Presence of E1 gene. To verify the presence of the E1 gene, the third or fourth leaf from the shoot apex can be used for protein extraction. Leaf samples can be harvested at 2-3 hours into the light period. Leaf tissues can be cut into approximately 1 cm² pieces and pooled for homogenization. An enzyme assay, SDS-PAGE, and western blot can be carried out as described previously (Z. Dai et al., Transgenic Res., 2000, 9: 43-54).

E1 Activity. To assess E1 activity, the third or fourth leaf from the shoot apex of transgenic plants can be harvested. One half of the leaf tissues can be sliced into 1 cm×2 cm pieces and the other half used for direct extraction as described above. About 0.15 g of leaf pieces can be vacuum-infiltrated with 50 mM MES (pH 5.5) twice each for 10 minutes at 20 in. of mercury. The infiltrated leaf pieces can be transferred into 1.5 mL microcentrifuge tubes and centrifuged at 350 g for 10 minutes to obtain fluid from the intercellular space. About 15-25 μL of intercellular fluid can be used for E1 activity measurement and 30-50 μL of intercellular fluid can be used for protein quantification.

### Example 2

### Enzymatic Performance and Stability of E1 Tobacco

The stability properties of leaf protein concentrates and associated E1 cellulase activity in E1-FLC transgenic tobacco were characterized.

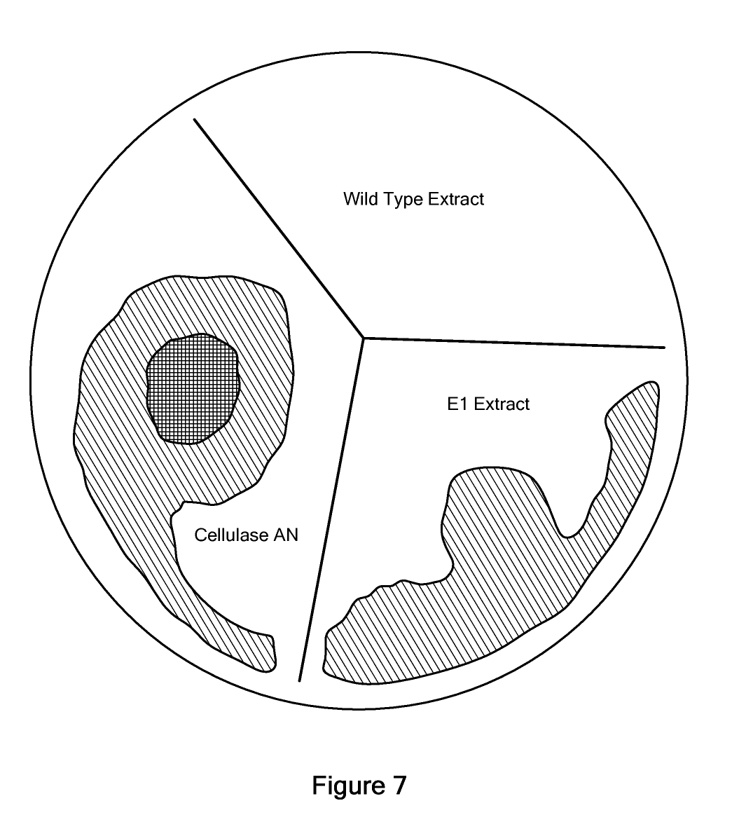

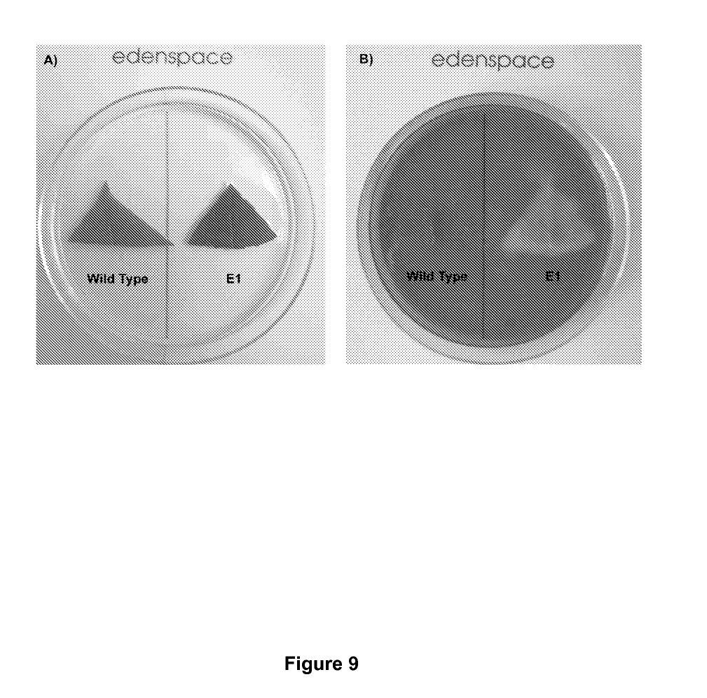

Leaf protein concentrates were prepared by macerating the tobacco leaves with ice in a blender at a ratio of 8:1 (w/1). Samples of these extracts were analyzed for cellulase activity using carboxy-methyl cellulose. As shown in FIG. 7, extract from E1 plants but not wild-type tobacco can hydrolyze cellulase, and the transgenic biomass itself shows cellulase activity (FIG. 9B). Samples of these concentrates were also subjected to various conditions to determine the effect of refrigeration at 2° C., pre-heating the sample at 90° C., acidification to pH 4.0 with lactic acid, and drying the plant material prior to addition to external cellulase (Spezyme CP from Genencor International, Inc., Palo Alto, Calif.). Nine combinations of these variables were studied in the presence and absence of added cellulase (25 μL cellulase per mL).

The stability/activity of the cellulase enzymes (both added/external and endogenous) within these concentrates were measured as a function of the hydrolysis of cellulose and glucose production. One (1) mL aliquots of each sample were added to 0.25 g of microcrystalline cellulose. The solution was brought to 10 mL in a pH 4.5 citrate buffer. The solutions were allowed to hydrolyze for 5 days, and the concentration of glucose was measured to assess cellulase activity of the sample. Long-term studies are underway to determine cellulase activity measurements for various times for up to 6 months. Hydrolysis of cellulose as a function of glucose concentration in the samples is presented in Table 2.

As is evident from Table 2, the addition of acid severely limits the activity of cellulase, which is greatest at about pH 4.9. Significantly, in all cases except those involving acid addition, the transgenic plant plus cellulase experiments produced more glucose that the control plus cellulase. This is strong evidence for the expression of cellulase activity in the transgenic tobacco. Some transgenic samples showed measurable glucose production even without added cellulase, whereas none of the controls showed such cellulase activity. Bacterial growth was seen in all room temperature acidified samples after only two weeks, and some growth was seen in one refrigerated sample after one month. As such, room temperature acidified storage appears unsuitable for long-term storage conditions.

TABLE 2
Concentrations of glucose (g/L) from transgenic and non-transgenic
tobacco after five (5) days of hydrolysis.

| | | | | Tobacco Genotype and Treatment | | | |
|---|---|---|---|---|---|---|---|
| | | | | No Added | | | |
| | | | | Cellulase | | Added Cellulase | |
| Pretreatment | | | | Control | E1/FLC | Control | E1/FLC |
| 2° C. | 90° C. | pH 4.0 | Dry | glucose (g/L) | | | |
| | | | | 0 | 0 | 3.13 | 3.72 |
| x | | | | 0 | 1.43 | 3.11 | 4.19 |
| | x | | | 0 | 0 | 3.44 | 4.50 |
| x | x | | | 0 | 0 | 3.08 | 5.74 |
| | | x | | 0 | 0 | 1.33 | 0.57 |
| x | | x | | 0 | 0 | 1.65 | 0.79 |
| x | x | x | | 0 | 0.40 | 0.82 | 1.57 |
| | | | x | 0 | 0.34 | 4.08 | 5.58 |
| | x | | x | 0 | 0 | 4.84 | 4.92 |

This experiment indicates that E1 cellulase activity in a concentrate of E1 plants is quite stable, suggesting that the plant juice can be used as a source of cellulase to hydrolyze non-transgenic plant biomass, or added back to the transgenic plants themselves after pre-processing steps such as high heat or acid treatment are completed that might otherwise inactivate the enzyme. The experiment also indicates that adding cellulases to E1 plants increases total glucose production compared to adding cellulases to non-transgenic plants. This is an important result since it suggests that simply using transgenic E1 plants with current external cellulase techniques can substantially increase ethanol yields.

As mentioned above, the “cellulase” enzyme system is complex and comprises various activities, while the transgenic E1 tobacco plant only expresses one of these activities, namely the endoglucanase. Three samples in the experiment described above showed glucose event in the absence of a complex cellulose complex, which is an encouraging result.

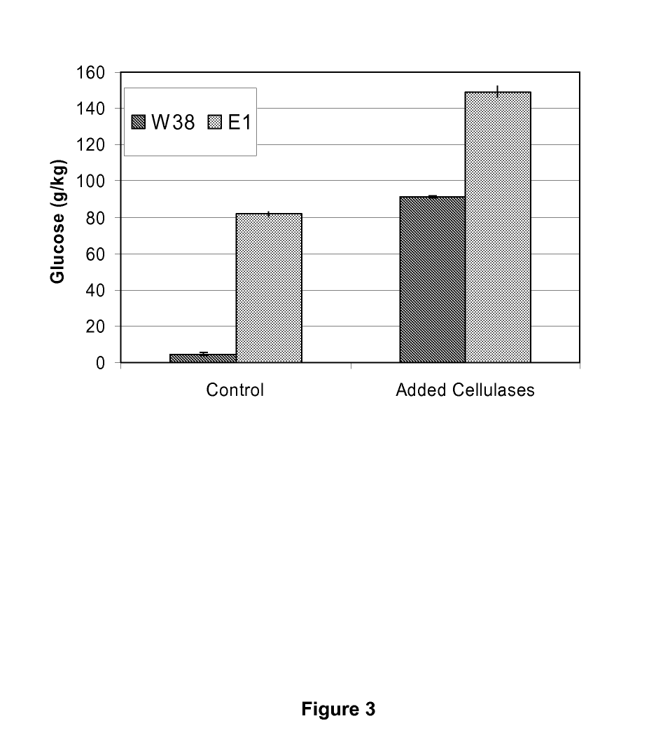

Adding additional members of the cellulase complex would be expected to increase hydrolysis of the E1 tobacco biomass. To test this hypothesis, an endoglucanase, glucoamylase and hemicellulase (obtained from BIO-CAT, Troy, Va.) were added to a non-transgenic tobacco and to an E1-FLC tobacco. As shown in FIG. 3, the results indicate that adding different enzyme types almost doubles glucose production in the transgenic tobacco, without chemical pretreatment. The E1-FLC tobacco was also found to produce a higher level of glucose than the E1-only tobacco used in the previous experiment.

These results suggest that creating additional plant genotypes expressing different members of the cellulase complex, such as ligninase and hemicellulase, could multiply the hydrolysis yield of E1 plants. The result also suggests that the E1 activity is not restricted to that of an endoglucanase. The increased production of glucose in the absence of external cellulases indicates that additional enzymatic hydrolysis of polysaccharides and disaccharides is occurring in a manner similar to that observed with exoglucanases, indicating that E1 may be what has been termed a “processive” endoglucanase.

### Example 3

### Transformation of Corn with E1-FLC

To develop a system for transforming corn, rice was used as cereal model plant system for transfer of the E1 and the bar genes. To do so, the pZM766E1-cat was inserted into pCAMBIA (purchased from pCAMBIA Co (Camberra, Australia) containing the bar selectable marker gene and the gus color indicator gene. The plasmid was obtained under a Material Transfer Agreement (MTA) from Dr. K. Danna of Colorado State University. The E1 transgenic rice plants showed the integration of all three transgenes (E1, bar and gus) by PCR. Furthermore, they showed E1 production as high as 24% total soluble proteins and enzymatic activity of the E1 transgene.

Several independent corn transgenic lines were then developed using biolistic bombardment. Lines showing confirmed integration, expression, enzymatic activity and accumulation of the transgene product inside of the apoplast were retained for further testing and development as described below.

Explant Preparation and Biolistic Bombardment of Corn. The Applicants produced multi-meristem apical shoot primordia for biolistic bombardment of a mixture of E1 and bar constructs. Corn was grown in greenhouses. Immature embryos were produced, cultured and callus lines were produced, and the immature embryo-derived callus lines were bombarded with a 1:1 ratio of a plasmid containing the E1 gene and one of the three plasmids, each containing the bar selectable marker gene.

For the E1 plasmid, the pMZ766E1-cat was selected because in Arabidopsis this construct produced the E1 enzyme up to 26% of the total soluble proteins (M. T. Ziegler et al., Mol. Breeding, 2000, 6: 37-46). This construct contains the strong promoter and enhancer and an apoplast targeting element. Corn multi-meristems and the immature embryo-derived callus lines were co-transformed with the pZM766E1-cat and either the pGreen or the pBY520, as each has its own potential advantages.

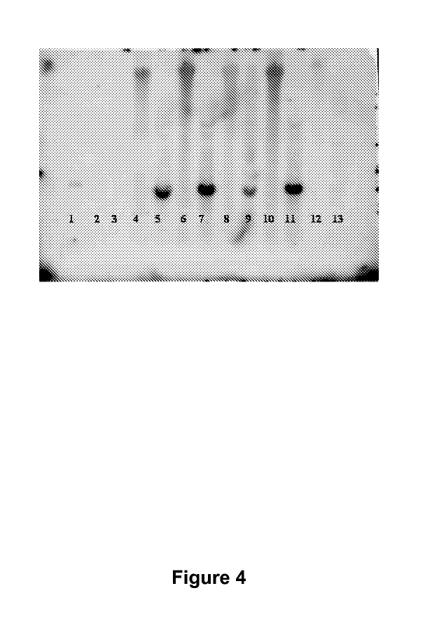

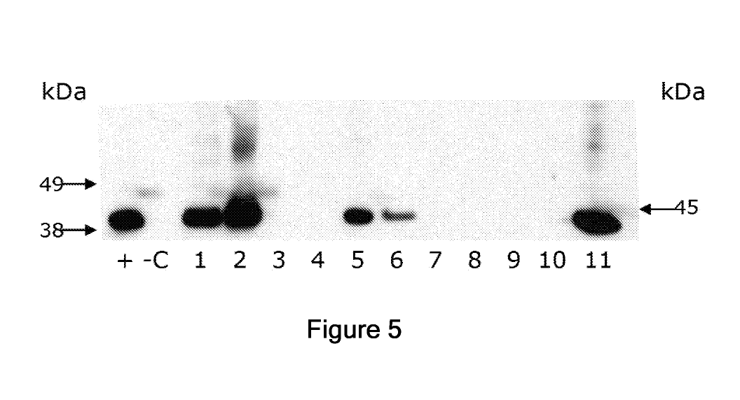

Confirmation Analysis of E1 Transgenic Corn. PCR was used on a few PPT-selected plantlets and confirmed the presence of the E1 and bar genes. Those plantlets which showed positive signals were selected for further studies. Although the copy numbers of E1 in corn plants using the gene unique site has not yet been determined, Southern blot analysis confirmed the stable integration of E1 transgene in several PCR positive corn lines (see FIG. 4). The translation of E1 transgene in corn was confirmed using western blots and compared to E1 translation in tobacco and rice (see FIG. 5).

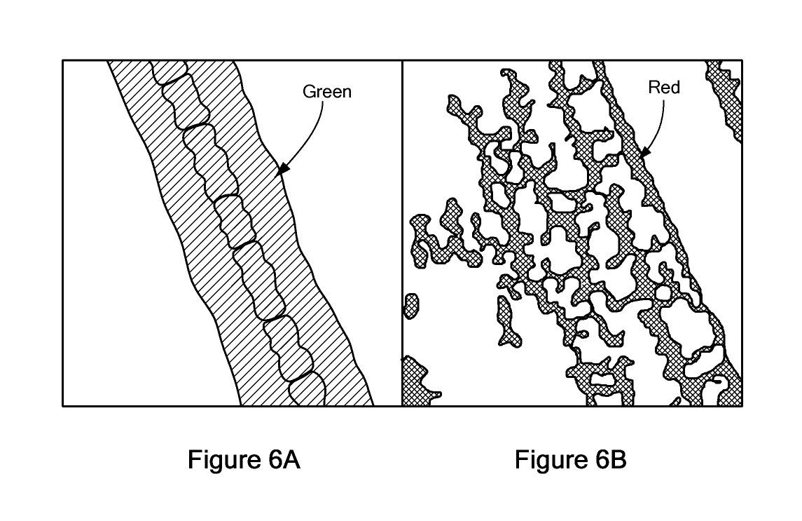

Preliminary Work on Apoplast Localization of E1 in Transgenic Corn. Based on previous experience with localization studies of other gene products (polyhydroxybutyrate) in corn via confocal microscopy, an E1 primary antibody and an appropriate secondary antibody were used to perform localization of E1 in transgenic corn tissue. Although most samples showed strong non-specific binding of the fluorescence conjugate to plant tissues, some samples showed possible localization of E1 in apoplast (FIG. 6). None of the plant lines generated as described herein appeared to show adverse growth effects from the non-specific binding of E1 in their tissues. Increased localization of E1 in apoplast will be pursued using several different blocking agents to reduce any potential non-specific binding.

### Summary of Results Obtained

The results obtained in the Examples reported herein provide strong support for the contention that tailoring crop plant traits can significantly improve biofuel yields and reduce biofuel production costs. Robust activity by a key cellulase enzyme, E1 endoglucanase, was demonstrated in transformed tobacco and corn. One E1 corn line has shown E1 production at more than 9% of total soluble proteins, a level approximately equal to the level of exogenous enzyme today added to cellulosic biomass for hydrolysis. Significantly, addition of exogenous enzymes led to higher glucose yields from E1 tobacco than non-transformed tobacco, suggesting that higher ethanol yields can be achieved simply by using today's hydrolysis techniques on E1 crop plants. The results obtained also showed that the FLC gene delays flowering in tobacco, as it had earlier been shown to do in Arabidopsis, a trait that is likely to be useful in bioconfinement of transgenes in bioenergy crops. FLC may also confer greater biomass. From the standpoint of co-production of crops for bioenergy and phytoremediation, the present results showed that E1-FLC plants can extract as much contaminant as the plants normally used in phytoremediation, and that two of the most contaminants, arsenic and lead, can be extracted from the harvested biomass at levels exceeding 95%, facilitating metals recycling as well as downstream bioenergy use of the hydrolyzed sugars.

### Example 4

### Increased Glucose Conversion from Transgenic Biomass

E1 and wild-type tobacco samples were air-dried to a moisture content of 16% and then subsets of the biomass were dilute acid pretreated (DAPT) using sulfuric acid according to standard techniques. Tobacco samples (W38 and E1 from Edenspace) were treated with 2% (w/w) sulfuric acid at 140° C. for 30 minutes in a Parr pressure reactor. The dry mass load of the samples in acid was 10% (about 50 g dry mass sample in 5000 g of dilute acid). Solid residues of pretreated samples were separated by centrifuging at about 4,500 rpm (˜6,500 g) for 20 minutes and washed four times with hot distilled water. Supernatants from each sample were pooled and brought to a volume of 2,000 mL with distilled water. A portion of diluted and thoroughly mixed supernatant (˜25 mL) was neutralized with calcium carbonate (˜0.12 g) to pH 6.0 and filtered through 0.2 μm syringe filter. Sugars released during pretreatment were analyzed using HPLC equipped with Phenomenex RCM column (7.8×200 mm) and RI detector with 0.60 mL/min deionized water as mobile phase and oven temperature 80° C. Solid residues were freeze dried and kept at room temperature for structural carbohydrate analysis and enzymatic hydrolysis. Untreated and DAPT samples underwent concurrent hydrolysis reaction with the addition of a cocktail of external cellulase enzymes (ACCELLERASE™ 1000). The reactions were incubated at 50° C. and glucose levels were determined at a variety of timepoints from 1 to 144 hours of incubation.

Enzymatic hydrolysis was conducted similarly to the National Renewable Energy Laboratory (NREL) protocol for enzymatic saccharification of lignocellulosic biomass (available at the web site whose address is http:// followed immediately by www.nrel.gov/biomass/analytical_procedures.html#lap-009) except that different buffer, dry mass load, and enzymes were used. Hydrolysis was carried out using a cellulose load of ˜3% (5-10% dry mass load), a buffer solution of 20 mM pH 5.0 sodium acetate buffer, and ACCELLERASE™ 1000 (0.24 mL/g of cellulose) enzyme from Genencor. ACCELLERASE™ 1000 has enzyme activities of 2500 CMC U/g and 400 pNPG U/g. Enzyme loads in this study were 600 CMC U and 96 pNPG U per gram of cellulose. Buffered hydrolysates were sealed in 120 mL serum bottles with 0.05% NaN₃ to inhibit possible microbial growth during hydrolysis. Hydrolysis was conducted in a 50° C. water bath with shaking at 100 rpm for 144 hours. 100 μL of supernatant was sampled from each hydrolysate after centrifuging at 2,500 rpm for 5 minutes. Sampled supernatants were mixed with 900 μL deionized water, filtered through a 0.2 μm filter, and analyzed for glucose, xylose, and arabinose using HPLC.

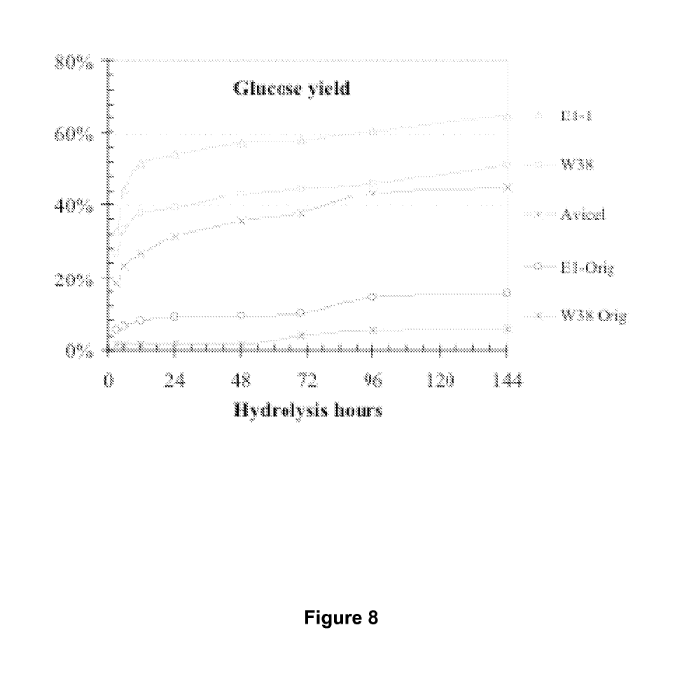

Glucose release kinetics for pretreated tobacco samples, non-treated original tobacco samples, and Avicel (Sigma cellulose, 20 μm) are shown in FIG. 8. Reactivity of cellulose in pretreated tobacco samples was higher than that of untreated tobacco. Reactivity of pretreated and untreated tobacco samples were both higher than that of commercial cellulose Avicel. Glucose yield from transgenic E1 samples was approximately 65% after 144 h hydrolysis (compare to 51% of wild type tobacco and 45% of Avicel). Glucose concentrations in hydrolysates were low (<2%) when cellulose load was 3% or less as in this Example. (For feasible ethanol production, a desirable glucose concentration is 10% and higher, meaning that cellulose load would be more than 10% and enzyme load would also be higher.)

The glucose released curve for untreated samples in FIG. 8 revealed that ACCELLERASE™ 1000 barely acted on untreated biomasses. Glucose released from wild type (W38) samples that were not pre-treated after 144 hours of hydrolysis only accounted for approximately 6% of the cellulose. Glucose released from transgenic E1 samples that were not pre-treated at the same time point was much higher (15.6%), suggesting that transgenic E1 may be active and exert effects in the tobacco sample even without pre-treatment.

### Example 5

### Codon Optimized Gene Sequences for Expression of Microbial Cellulases in Plants

As already mentioned above, a method was developed to modify microbial genes for increased expression in plants. A composite plant codon usage table was constructed from the analysis of the sequenced genomes of Zea mays, Arabidopsis thaliana, and Nicotiana tabacum. The codon usage of each of those genomes were averaged together to obtain a composite codon usage from monocot and dicot plants, and this composite table was used as a template to modify microbial DNA sequences so that the microbial sequences have a codon usage better suited for expression in plants.

For increased transcriptional expression of the E1 endoglucanase from Acidothermus cellulolyticus (GenBank accession numbers U33212 (nucleotide sequence) and AAA75477 (amino acid sequence)) in plants, the microbial sequence of the gene was optimized using a composite plant codon usage table. The average codon usages in Zea mays and Arabidopsis thaliana were obtained from the Kazusa Codon Usage Database (http://www.kazusa.or.jp/codon/) and averaged together to produce the composite plant codon usage table. With optimization, the E1 sequence used for transformation into plants had SEQ ID NO. 7 as follows:

ATGGGCTTCGTTCTCTTTTCTCAACTCCCCTCCTTCCTTCTCGTTTCTAC
TCTTCTTCTGTTCCTCGTAATCTCACATTCATGTCGCGCCGCAGGCGGTG
GTTATTGGCATACTTCCGGCAGAGAGATACTTGACGCTAACAACGTTCCC
GTACGCATCGCTGGTATTAATTGGTTTGGTTTCGAGACGTGCAATTATGT
CGTTCACGGTCTTTGGTCTCGCGATTACCGTTCAATGCTGGATCAAATAA
AATCTCTCGGCTACAATACAATTCGCCTTCCCTACTCGGATGATATCTTG
AAACCAGGTACTATGCCCAACTCAATTAATTTTTATCAAATGAATCAAGA
CCTTCAAGGCCTGACATCCCTTCAAGTTATGGACAAGATAGTTGCTTACG
CAGGACAAATAGGACTTAGGATTATTCTCGACAGACACAGACCCGACTGC
TCTGGCCAAAGCGCTCTCTGGTATACTTCATCCGTCAGTGAAGCTACCTG
GATCTCTGATCTTCAAGCACTTGCCCAACGTTACAAAGGAAACCCTACTG
TTGTTGGTTTCGATCTTCACAACGAACCTCACGATCCCGCCTGTTGGGGC
TGCGGAGACCCATCTATTGACTGGAGATTGGCCGCCGAACGTGCTGGCAA
CGCAGTGCTGTCCGTAAATCCCAACCTGCTTATATTTGTCGAAGGCGTAC
AATCCTATAATGGTGACTCCTATTGGTGGGGCGGAAACTTGCAAGGCGCA
GGACAGTATCCAGTTGTCCTCAATGTCCCGAATCGTCTCGTTTACTCAGC
ACACGACTACGCTACTTCCGTATACCCGCAAACTTGGTTCAGCGACCCGA
CATTCCCAAATAACATGCCCGGTATCTGGAATAAAAATTGGGGTTATCTC
TTCAACCAAAACATCGCGCCCGTTTGGCTTGGAGAATTCGGCACTACTCT
GCAATCGACTACAGACCAAACTTGGCTCAAGACTCTTGTCCAGTACCTCA
GACCTACAGCACAATACGGAGCAGACTCATTTCAATGGACATTTTGGTCC
TGGAACCCGGATTCTGGCGATACTGGCGGTATTCTTAAAGATGATTGGCA
AACTGTTGACACTGTCAAGGACGGCTACCTCGCACCTATCAAATCCTCGA
TATTCGATCCAGTTGGC

### Example 6

### Processing of Biomass from Transgenic Corn Expressing Low Levels of E1

The present Example illustrates that the production of E1 in corn stover, even at very low levels, leads to increases in glucan conversion rates when compared to conversion rates of stover from untransformed (WT) corn. In the present Example, E1 was expressed in corn at levels less than about 0.1% TSP and led to increases in glucan conversion rates between about 3% and about 20% compared to untransformed corn.

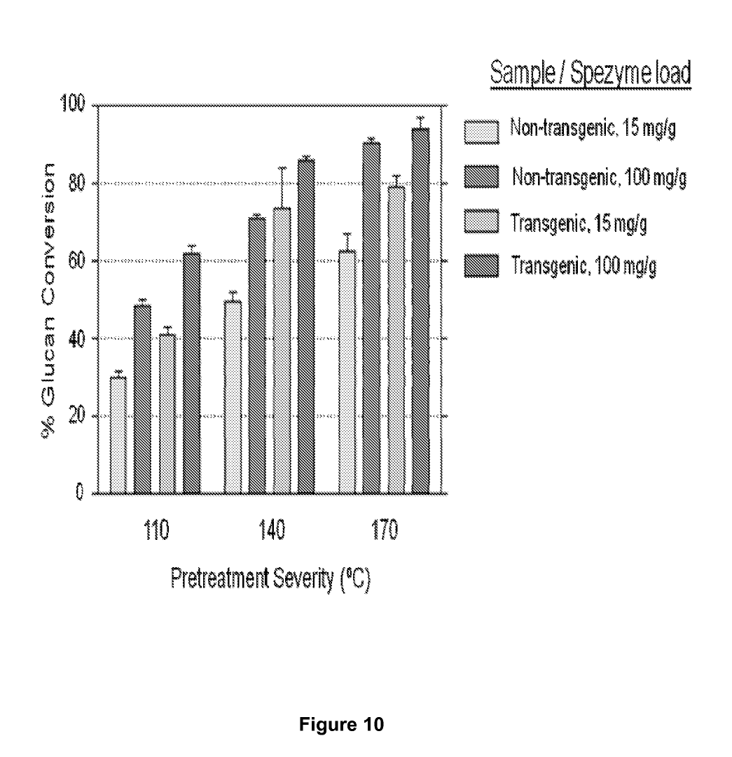

Dried corn stover was ground, then acid pretreated for ten minutes in 0.5% sulfuric acid at three temperatures to cover a range of severity. The resulting slurry was brought to a neutral pH, and then hydrolyzed with either a low (15 mg Spezyme/g biomass) or high (100 mg/g) concentration of enzyme. All reactions were supplemented with β-glucosidase to prevent cellobiose inhibition. Glucose concentrations in the hydrozylate were analyzed by HPLC and compared against theoretical yields. Glucan conversion rates are presented in FIG. 10 as percentages of theoretical yields.

In all conditions tested in this Example, E1 stover yielded higher levels of glucan conversion than WT stover. For stover pretreated at 140° C., the yield from E1 stover processed in the presence of low amounts of externally applied enzyme polypeptide was equivalent to the yield from WT stover processed in the presence of high amounts of externally applied enzyme polypeptide. Thus, E1 expression led to a reduction of almost 90% of the cellulases needed for hydrolysis. For stover processed in the presence of high amounts of externally applied enzyme polypeptide, the yield from E1 stover pretreated at 140° C. was comparable to the yield from WT stover pretreated at 170° C. Thus, E1 expression allowed lower pretreatment temperatures for a similar level of hydrolysis. Without additional cellulases, glucan conversion of E1 stover was equivalent to that of WT stover (about 1.5%), indicating that starting glucose concentrations in the biomass were similar but that the presence of E1 enabled more efficient enzymatic hydrolysis.

These results strongly suggest that current loading levels of externally applied enzymes can be substantially reduced by using glycozymes expressed in the plant. Furthermore, using glycozymes expressed in the plant may lower pretreatment temperatures required to achieve a particular level of hydrolysis.

### Example 7

### Tempering Prior to Pretreatment Improves Digestibility of Transgenic Biomass

Standard dilute acid pretreatment conditions may denature activity of even thermotolerant enzymes. In the present Example, we modified post-harvest processing procedures to favorably engage activity of transgene-encoded enzymes. Such modifications to industrial processing regimes, which we have categorized as “tempering,” led to improved digestibility of transgenic biomass.

Standard pretreatment and enzyme hydrolysis analyses, even at the laboratory scale, require hundreds of grams of biomass—a scale that may hinder the utility of such analyses during the early stages of screening of transgenic plants, when biomass availability may be limited. We employed additional methods requiring less starting material to supplement data available from standard pretreatment and hydrolysis studies.

We developed and used tempering protocols to determine their effects on digestibility. Digestibility of tempered biomass was measured using a modified in vitro dry matter digestibility (IVDMD) assay. IVDMD is based on the principle that the more digestible a material is, the more mass loss will be observed (on a dry matter basis) when exposing that material to hydrolytic enzymes in vitro. Milled material (˜50 mg) was transferred to each tube, and the weight of the material was recorded. Extractable compounds were removed from samples using a standard ethanol-acetone extraction procedure and dried to completeness in a fume hood. The dry weight of the sample was recorded. The weight after solvent extraction represented starting dry weight of the sample. Samples were then treated according to their experimental group.

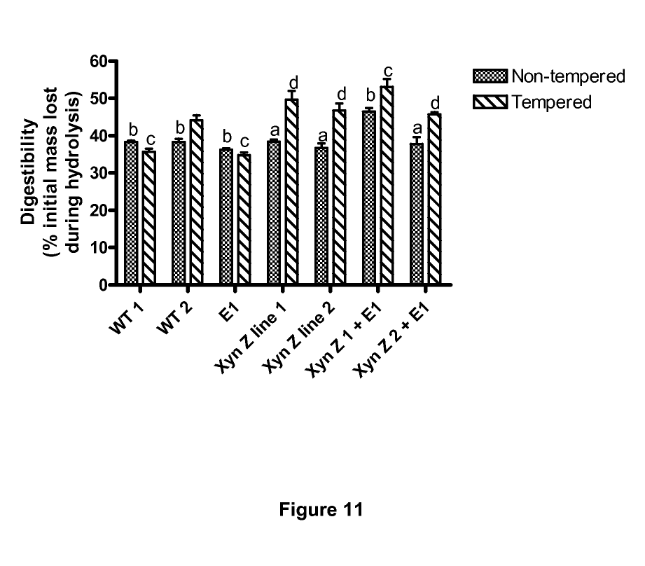

In FIG. 11, samples in the tempered group were reconstituted in sodium acetate buffer, pH 5.0 containing 5 mM calcium chloride and incubated at 85° C. for 24 h while samples in the non-tempered group were kept in their dry state. After the 24 h tempering period, tempered samples were centrifuged and supernatants were removed. Samples in the tempered and non-tempered groups were reconstituted in buffer (sodium acetate pH 5.0 and 5 mM CaCl₂) containing Novozymes Celluclast 1.5L and A188 β-glucosidase. Samples were incubated at 50° C. for 24 h and then rinsed extensively with water to remove hydrolyzed materials liberated during the 24 h hydrolysis period. Samples were dried to completeness in a dehydrator and the final dry weight of the sample was recorded. The amount of mass lost during the enzyme digestion was determined by subtracting the final sample weight from the starting weight. The “digestibility” of a sample was determined by calculating percentage of mass lost during the IVDMD procedure.

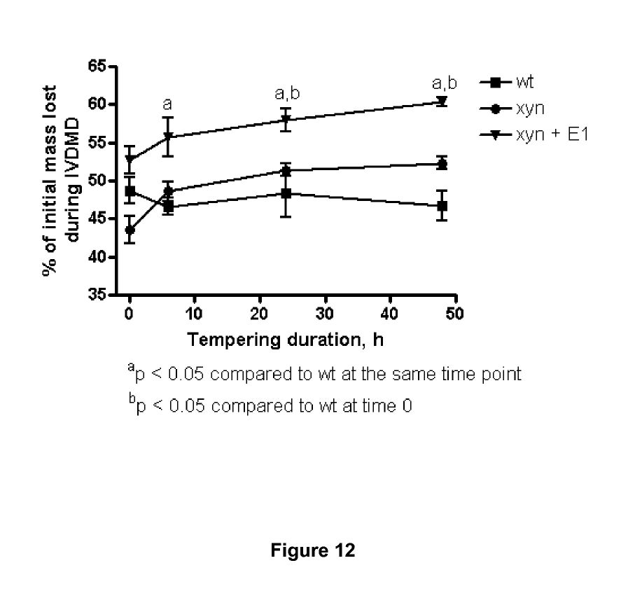

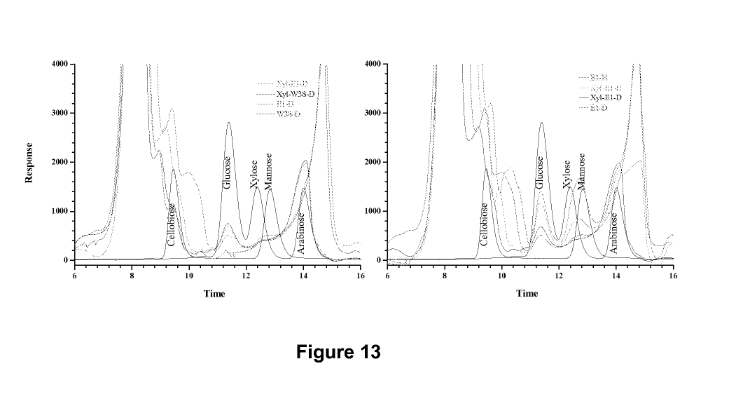

Transgenic tobacco biomass was incubated at 85° C. before digestion with a commercial enzyme cocktail (Novozymes Celluclast 1.5L). Pre-digestion incubation for 24 hours preferentially enhanced digestibility (FIG. 11). Digestibility-enhancing benefits of tempering were observed even with shorter tempering periods (e.g., 5 hours) (FIG. 12.) To analyze the sugars released during tempering, supernatants from biomass slurries after tempering for 15 hours were collected and analyzed by HPLC. HPLC profiles revealed that most of the released sugars were water soluble oligosaccharides (FIG. 13, left panel.)

This Example demonstrates that tempering by incubating biomass at a high temperature prior to pretreatment improves digestibility.

### Example 8

### Tempering by Ensilement Increases Glucose Conversion

Bioconversion of lignocellulosic biomass from its unprocessed form as a plant in the field to final commercial products typically involves for processing phases: harvest, storage, pretreatment, and hydrolysis to fermentable sugars. In the present Example, we developed and tested another tempering protocol that allowed integration of harvesting and storage phases with pretreatment and hydrolysis.

Ensilement is an effective method of long-term biomass preservation and storage. An object of ensilement is to preserve harvested biomass by anaerobic fermentation without losing feed quality. Without wishing to be bound by any particular theory, we recognized that it would be advantageous if the biomass could be conditioned during the preservaton period to aid downstream bioconversion. Recent data from Edenspace suggests that expression of endoplant enzymes and early activation of those enzymes during storage can complement and subsequently reduce pretreatment requirements, creating favorable conditions to reduce total production costs.

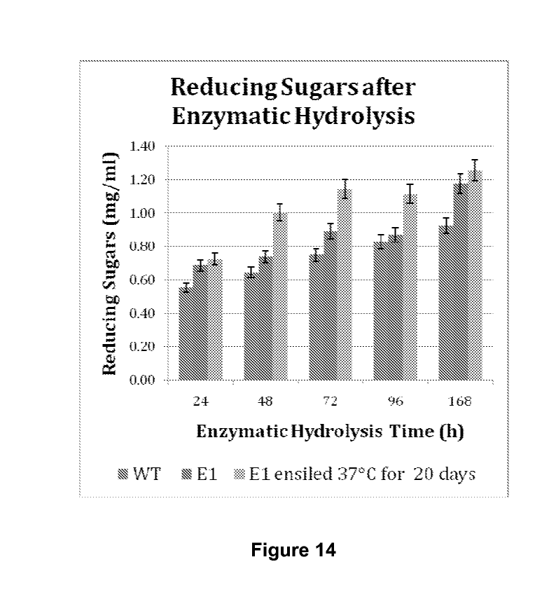

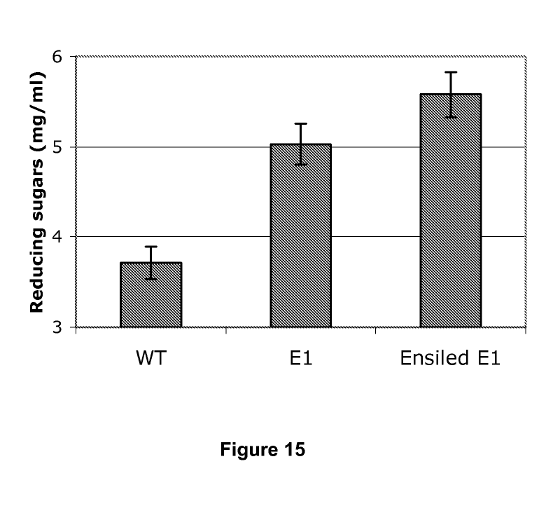

E1 transgenic and non-transgenic tobacco samples were ensiled at various times and temperatures. Ensilement of E1 tobacco led to significant increases in the amount of sugars released by Novozymes Celluclast 1.5L as compared to unensiled tobacco (FIG. 14). Tempering by ensilement also extended benefits to samples undergoing standard dilute acid pretreatment. Ensilement of E1 transgenic tobacco biomass led to a ten percent increase (as compared to unensiled tobacco) in sugar release following pretreatment and enzyme hydrolysis (FIG. 15).

These results demonstrate that ensilement has the potential to reduce pretreatment requirements and lower the cost of bioprocessing.

### Example 9

### Edenspace Biorefinery Process for Converting Biomass to Biofuels

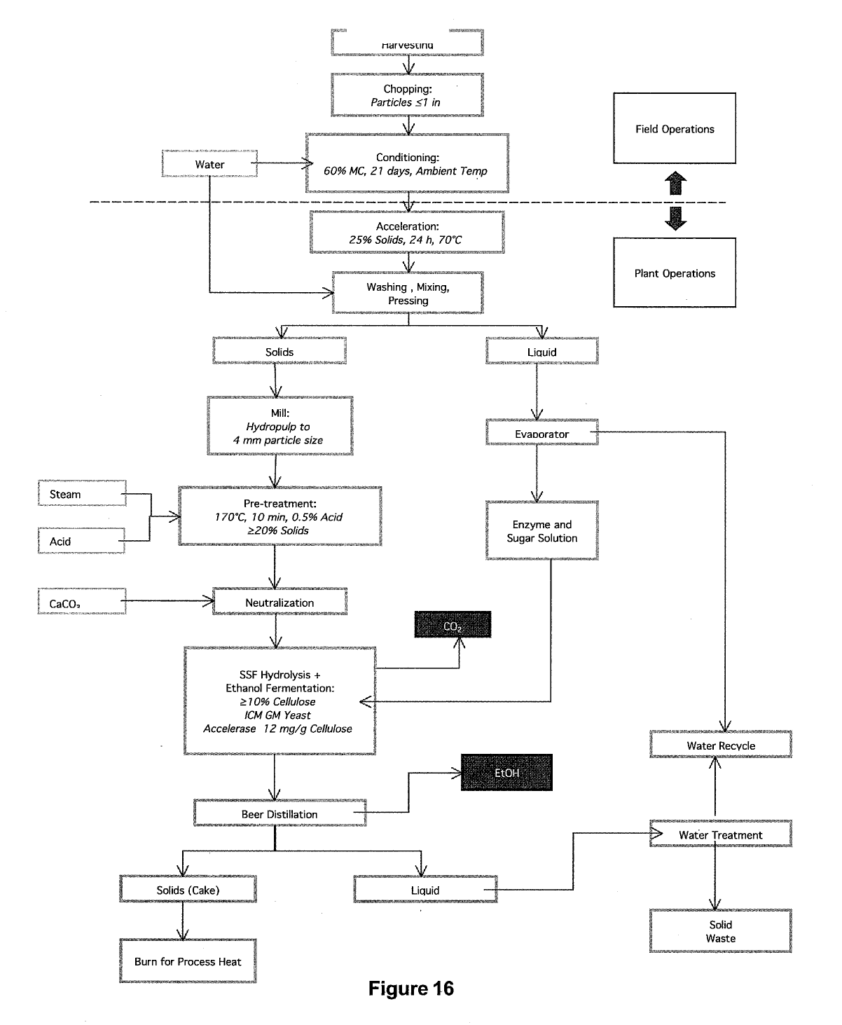

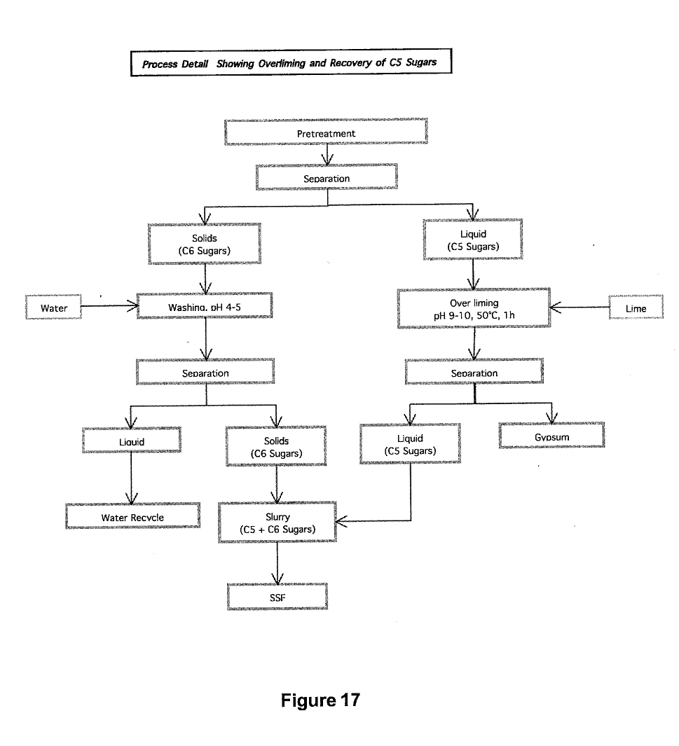

The Edenspace Biorefinery Process is uniquely optimized for production of biofuels such as ethanol from the biomass of Edenspace-proprietary Energy Crops such as Energy Corn™, Energy Sorghum™, Energy Switchgrass™ and Energy Poplar™. This Process is diagrammed in FIGS. 16 and 17 and results in superior products and lower cost of production.

Field operations shown in FIG. 16 begin at harvest, when the biomass is chopped to a suitable size. The chopped biomass is then stored for a convenient period of time in the step labeled “conditioning.” During conditioning, the biomass is stored in full sun under low oxygen conditions, either in large “ag-bags” or in bales. These conditions stimulate a natural process in which biomass temperatures rise to a level that activates Edenspace endoplant enzymes and initiates biomass deconstruction.

The conditioning operation can be as short as 21 days or as long as several months. The conditioned biomass is delivered to the biorefinery on demand. Plant operations begin when the biomass is delivered to the biorefinery. At the biorefinery, the biomass is adjusted with water to 25% solids and subjected to acceleration, a unique operation in which the temperature is raised to 70° C. for 24 hours. In this step, Edenspace endoplant enzymes are activated and permitted to accelerate biomass deconstruction

Acceleration is followed by another unique operation in which the biomass is washed, mixed and pressed to extract a high-value liquid (or “crude extract”) that contains soluble sugars and enzymes. Sugars and enzymes recovered in this liquid fraction are concentrated through evaporation and combined with cellulose, hemicelluloses and other carbohydrates in the step labeled SSF. Following washing, mixing, and pressing, the wet solids are milled to an average particle size of 4 mm using a hydropulper. Milling wet, pre-conditioned solids through use of a hydropulper produces a more uniform milled product at lower net energy cost. Milled solids are then moved into the pretreatment operation. In pretreatment, the solids content is adjusted to approximately 20%, sulfuric acid is added to 0.5%, and the temperature is raised to approximately 170° C. for 10 minutes. These pretreatment conditions are milder than standard conditions, as they have been optimized for use of biomass from Edenspace-proprietary Energy Crops that has been subjected to conditioning and acceleration. Such biomass typically requires less heat and acid for liberation of cellulose, hemi-cellulose and other carbohydrates suitable for saccharification and fermentation. Edenspace pretreatment de-couples lignin from valuable sugar polymers such as cellulose and hemicelluloses without producing lignin degradation products that are known to inhibit fermentation. In addition, Edenspace pretreatment is conducted at a temperature below the melting point of lignin, which minimizes fouling and contamination surfaces in Biorefinery vessels and pipes.

After pretreatment, the biomass mixture is neutralized by adding calcium carbonate (lime). The neutralization operation is shown in more detail in FIG. 17, labeled ‘Process Detail.’ The neutralization step requires less calcium carbonate than standard operations, because less sulfuric acid was used in Pretreatment. This also means that less gypsum is produced. The use of reduced amounts of acid also facilitates “over-liming,” a step in which the pH is raised above the neutral point to a more alkaline point in the range of pH 9-10. This allows for efficient recovery of C5 sugars, which are added to the SSF reaction, increasing overall process efficiency and boosting net yields.

After pretreatment and neutralization, the biomass is subjected to simultaneous saccharification and fermentation, the operation labeled SSF in FIG. 1. At this stage, the pretreated biomass contains mostly cellulose, together with some hemicellulose and other less abundant carbohydrate polymers. The soluble phase contains pentose sugars and enzymes added from two sources: washing, mixing and pressing of Energy Crop biomass, as well costly exogenous enzymes. Use of Edenspace Energy Crops reduces the need for costly exogenous enzymes. During SSF, enzymes break down cellulose and any remaining hemicelluloses, and free sugars are converted to ethanol through fermentation. The fermentative microorganism in this Example is a proprietary yeast that is efficient for conversion of both hexoses and pentoses to ethanol. Following SFF, the Distillation Operation and final recovery of solids are standard. The entire process makes maximum use of internal recovery and re-use of water and heat.

### Example 10

### Expression of Cellulases Improve the Conversion of Lignocellulosic Biomass from Transgenic Corn Stover

Example 6 illustrated that production of E1 corn stover, even at very low levels, leads to increases in glucan conversion rates when compared to conversion rates of stover from untransformed (WT) corn. The present Example further illustrates enhanced glucan conversion of lignocellulosic biomass from transgenic corn stover.

Biomass from a segregating population of transgenic plants containing a transgene expressing either E1 endoglucanase or CBHE cellobiohydrolase I was trait tested for the presence of the selectable marker using a commercial trait testing kit. Each plant in the field was tagged as trait negative or positive according to the absence or presence, respectively, of the selectable marker. Plants were allowed to reach senescence in the field before cobs and stover were harvested. Stover was further dried in a forced air dryer before milling to 1 mm size using a Wiley mill.

Milled corn stover from trait positive plants expressing E1 (ED112.02D (1) Pos) along with its respective trait negative control (ED112.02D(1) Neg) was subjected to dilute acid pretreatment (0.5% v/v sulfuric acid, 10 min) using three different pretreatment temperatures: 150° C., 170° C., and 190° C. Following pretreatment, samples were neutralized, rinsed, and then subjected to enzymatic hydrolysis with two levels of enzyme (0.2 and 0.5 mL ACCELLERASE™ 1000/g glucan) at 50° C. for 72 hrs. Extracts were analyzed for glucose content using a commercial glucose oxidase-based assay kit or by ion chromatography.

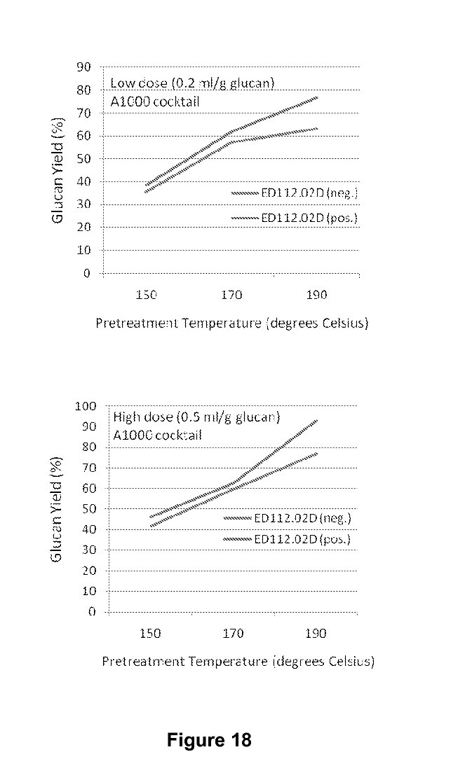

As shown in FIG. 18, the glucan conversion efficiency of corn stover expressing the E1 endoglucanase (ED112.02D (1) Pos) was considerably greater than its trait negative control when dilute acid pretreatment was conducted at a temperature of 190° C.

Milled corn stover from trait positive plants expressing the gene encoding the CBHE cellobiohydrolase I (ED122.05A Pos) along with its respective trait negative controls was subjected to dilute acid pretreatment using the following conditions: 190° C., 10 min, 0.5% v/v sulfuric acid. Following pretreatment, samples were neutralized, rinsed, and then subjected to enzymatic hydrolysis with two levels of enzyme (0.2 and 0.5 mL ACCELLERASE™ 1000/g.glucan) at 50° C. for 72 hrs. Extracts were analyzed for glucose content using a commercial glucose oxidase-based assay kit.

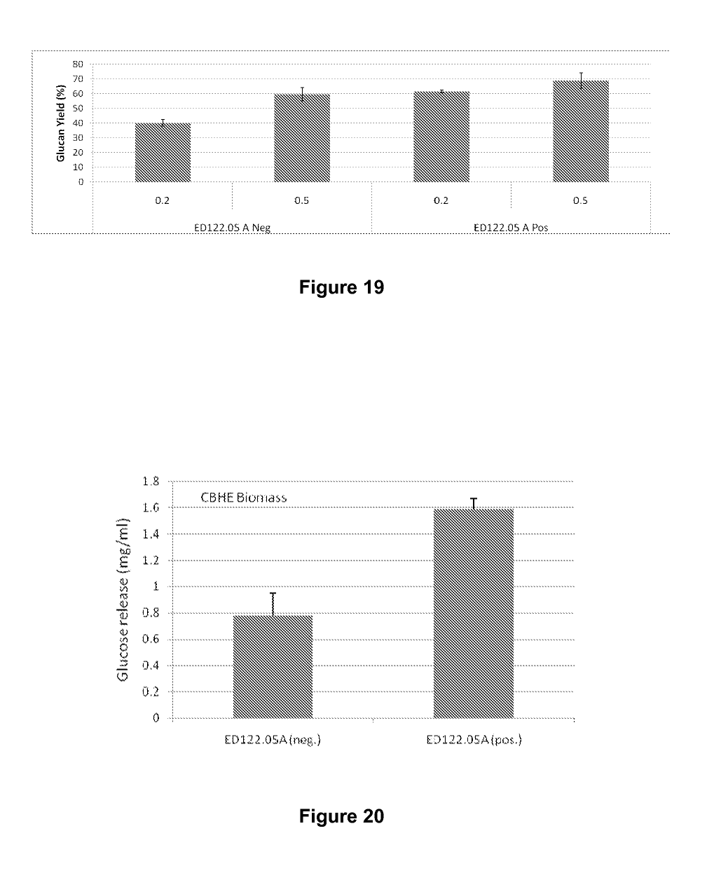

As shown in FIG. 19, biomass from trait positive plants (ED122.05A Pos) had a greater level of glucan conversion when compared at the same level of ACCELLERASE™ 1000 enzyme loading (0.2 mL/g or 0.5 mL/g glucan) to its matched trait negative genetic control (ED122.05A Neg). These results also demonstrate that expression of CBHE exoglucanase (ED122.05A Pos) makes it possible to achieve the same level of glucan conversion as its genetic control (ED122.05A Neg) using 60% less ACCELLERASE™ 1000 dose (0.2 mL/g versus 0.5 mL/g).

### Example 11

### Tempering by Acceleration Enhances Release of Glucose from Transgenic Biomass

Example 7 illustrated that tempering by hydrating dried transgenic biomass and incubating it in heat prior to pretreatment (e.g., “acceleration”) improved digestibility of transgenic biomass. The present Example illustrates how such acceleration leads to substantial release of glucose.

Biomass from the first generation of plants derived from segregating population seeds from regenerated transgenic plants (transgenic for CBHE enzyme) was trait tested for the presence of the selectable marker using a commercial trait testing kit. Each plant in the field was tagged as trait negative or positive according to the absence or presence, respectively, of the selectable marker. Plants were allowed to reach senescence in the field before the cobs and stover were harvested. Stover was further dried in a forced air dryer before milling to 1 mm size using a Wiley mill.

To temper the biomass prior to pretreatment, we subjected the biomass to acceleration to activate the CBHE enzyme on the plant cell wall and reduce recalcitrance. Milled corn stover from trait positive plants expressing genes encoding CBHE cellobiohydrolase I (ED122.05A (pos.)) along with its trait negative genetic control (ED122.05A (neg.)) was reconstituted as a slurry (10% solids) in 50 mM sodium citrate (pH 5.0) and incubated at 70° C. for 24 hrs to engage activity of the CBHE exoglucanase. After acceleration, samples were centrifuged and glucose levels in the supernatant were determined using a glucose oxidase-based assay kit.

As seen in FIG. 20, acceleration of biomass from CBHE transgenic plants (ED122.05A (pos.)) produced twice as much glucose following enzymatic hydrolysis as compared that produced from similarly-treated biomass from the trait negative genetic control (ED122.05A (neg.)).

### Example 12

### Tempering of Corn Stover and Grain by Ensilement

Example 8 illustrated development and testing of a tempering protocol involving ensilement, which allowed integration of harvesting and storage phases with pretreatment and hydrolysis. Heat generated through ensilement can significantly increase enzyme activity. The present Example illustrates that enzymatic activity is preserved in corn stover and grain after 90 days of ensilement.

Stover and grain from ensilement-stage corn (30% moisture content) plants (E1 endoglucanase trait positive and negative) were collected. Ears were separated from stalks, stover was processed through a wood chopper, and grain was removed from cobs. As appropriate, water was added to chopped stover samples to bring the moisture content of all samples to 30%. Grain was used without further adjusting moisture content. Quadruplicate samples of chopped stover from E1 trait positive and negative, designated TR1-4 and NT1-4 respectively, were loaded into airtight capsules, sealed, and stored at 21° C. for 90 days to allow microbial ensilement to occur. Duplicate samples of grain from E1 trait positive and negative, TRG1-2 and NTG1-2, respectively, were similarly loaded into airtight capsules and allowed to ensile for 90 days.

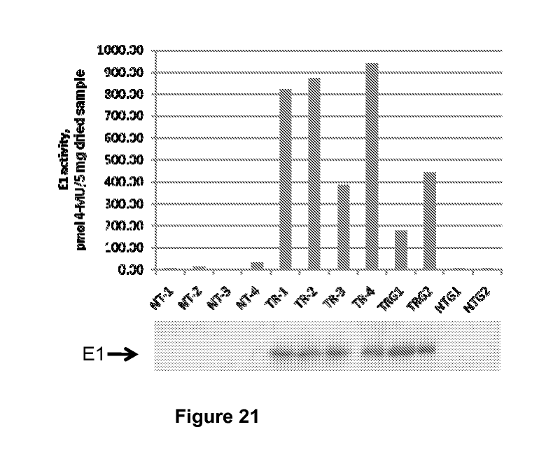

After 90 days, capsules were unsealed and samples were collected and dried to completeness in a 45° C. laboratory drying oven. Stability of E1 protein and activity were determined by Western blotting and enzyme activity assay respectively. For Western blotting, 5 mg of dried material were boiled in Laemmli buffer for five minutes and centrifuged. Supernatant was loaded onto a 10% SDS-PAGE gel. Following electrophoresis to resolve proteins in the sample, proteins in the gel were electrophoretically transferred to a PVDF membrane and subsequently immunoblotted with a monoclonal anti-E1 primary antibody and appropriate horseradish peroxidase (HRP)-labeled secondary antibody. Immunoreactive bands were visualized by an HRP-catalyzed reaction that converts a non-colored substrate into a purple colored precipitate in situ. (See FIG. 21, bottom panel).

E1 activity was measured by incubating 5 mg samples of dried material in a reaction mixture containing 50 mM sodium acetate, pH 5.0 and 100 μM 4-methylumbelliferyl cellobioside (MUC). Samples were incubated at 85° C. for 1 hour. At the end of the incubation period, an equal volume of 1 M sodium carbonate was added, an aliquot of the mixture was transferred to a black 96-well plate, and release of 4-methylumbelliferone (4-MU) was measured with a fluorescent plate reader (Excitation wavelength, 355 nm; Emission wavelength, 450 nm) (FIG. 21, top panel). These results demonstrate that E1 protein and activity are stable after a 90-day ensilement.

### Example 13

### Tempering of Tobacco Biomass by Ensilement

The present Example illustrates that tempering of tobacco biomass by ensilement leads to increased release of reducing sugars.

Dried tobacco biomass samples expressing (‘E1’) or lacking (‘WT’) the E1 endoglucanase were reconstituted in 100 mM sodium citrate (pH 4.8) to a moisture content of fifty percent and loaded into sealed plastic bags. To obtain an anaerobic environment, air was removed by applying a vacuum to a port on each bag. Following incubation of the bags at 37° C. for 20 days to allow ensilement of the biomass, 1 gram samples (wet basis) were mixed with 10 mL of solution containing 0.1 M sodium citrate (pH4.8), Celluclast 1.5 L (a Novozymes cellulase mixture), and Novozyme 188 (a source of β-glucosidase). Samples were incubated at 50° C. for 24-72 hours. Reducing sugars in liquid phase collected at 24, 48, and 72 hours were measured using the dinitrosalicylic acid method.

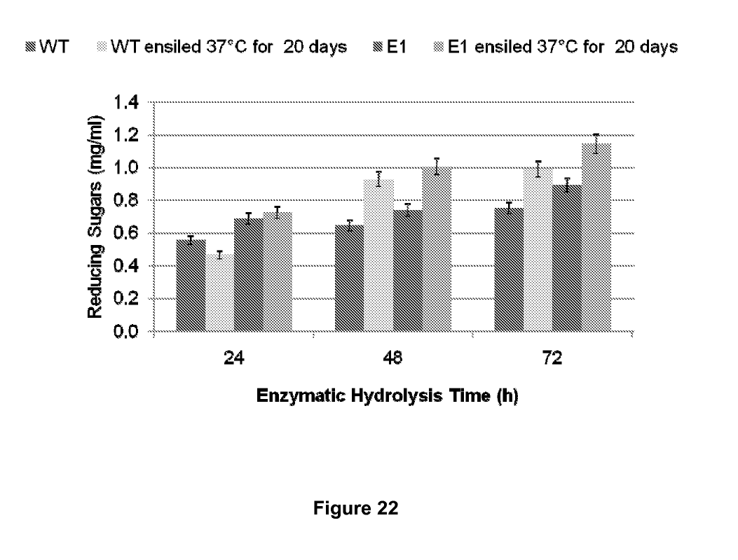

As seen in FIG. 22, more reducing sugars were released from unensiled tobacco containing the E1 endoglucanase than from wild-type tobacco following enzymatic hydrolysis with Celluclast 1.5L and Novozyme 188. (Compare the third bar in each time period group to the first bar in each time period group.) This enahncement was also observed with ensiled biomass. When enzymatic hydrolysis with commercial enzyme cocktails was performed for 48 or 72 hours, a clear increase in the reducing sugars was observed in samples ensiled prior to enzyme hydrolysis. (Compare the second bar to the first bar in each time period group and the fourth bar to the third bar in each time period group). There was also a clear increase in reducing sugars from ensiled E1 biomass compared to WT biomass. The combination of the E1 transgene, ensilement, and a 72 hour enzymatic hydrolysis period with commercial enzyme cocktail proved to be superior to other combinations in this experiment in terms of increasing the release of reducing sugars from biomass.

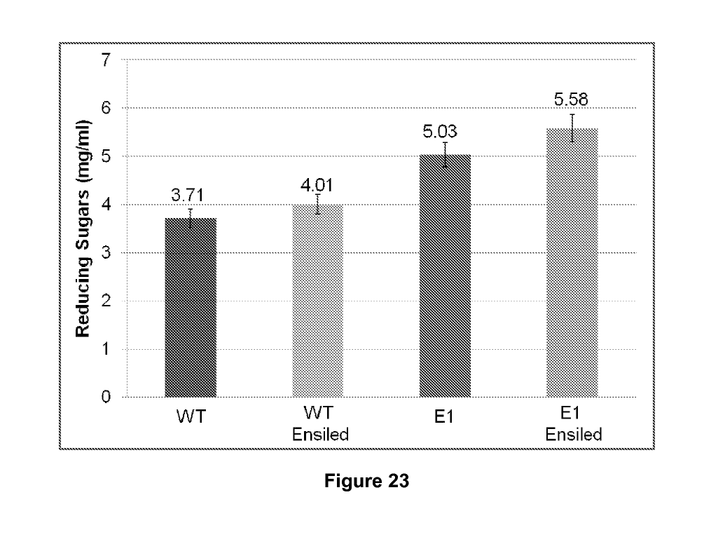

Benefits of ensilement were also observed when samples were subjected to dilute acid pretreatment (121° C., 60 min, 0.5% v/v) and enzyme hydrolysis using Celluclast 1.5L and Novozyme 188. As compared to wild-type tobacco, transgenic E1 tobacco showed a 30% increase in amount of reducing sugars released during enzyme hydrolysis (FIG. 23). When compared to unensiled E1 tobacco, ensilement of E1 tobacco led to a ten percent increase in sugar release following pretreatment and enzyme hydrolysis. These results demonstrate that ensilement has the potential to reduce pretreatment requirements and lower the cost of existing bioprocessing regimes.

### Example 14

### Tempering by Combined Ensilement and Acceleration

The present Example describes testing of a tempering process that combines ensilement and accelaration.

Corn stover, switchgrass, and poplar represent cellulosic ethanol feedstocks with high commercial potential. To demonstrate the ability of tempering to enhance the performance of custom engineered feedstocks, laboratory experiments were conducted that compare standard dilute sulfuric acid pretreatment, the “Standard process,” to a milder, less cost intensive pretreatment process, the “Edenspace process”, designed to take advantage of the properties of engineered Edenspace crops. Samples subjected to the Edenspace process were tempered by ensilement under high moisture content conditions (60% MC) and then acceleration for 24 h at 70° C. prior to dilute acid pretreatment as indicated. Standard process conditions did not include ensilement or acceleration. Pretreatments for both processes were performed in an Advanced Microwave Biodigester with conditions described in Table 3.

TABLE 3
Tempering conditions used for bench scale testing

| | Ensilement + | Reaction | Pretreatment | Sulfuric | Total | |
|---|---|---|---|---|---|---|
| | 24 h | Time | Temperature | Acid | Solids | |
| Process | Acceleration | (min) | (° C.) | (% (v/v)) | (%) | Cooling Type |
| Standard | No | 10 | 190 | 0.5 | 20 | Flash |
| Edenspace | Yes | 10 | 175 | 0.5 | 20 | Flash |

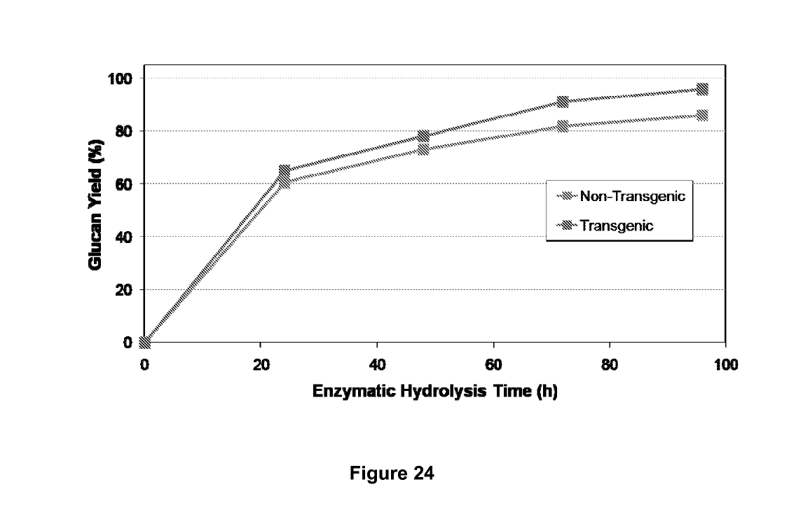

E1 switchgrass and non-transgenic control switchgrass were tempered and pretreated under Edenspace process conditions (Table 3). After hydrolysis with Accelerase 1000 (Genencor) at a load of 0.2 mg/g, glucan samples were analyzed by HPLC to determine glucose levels. A net 10% increase of glucan conversion yield was observed for the E1 switchgrass compared to conventional switchgrass following 96 h of hydrolysis, achieving nearly complete glucan conversion of the transgenic biomass (FIG. 24).

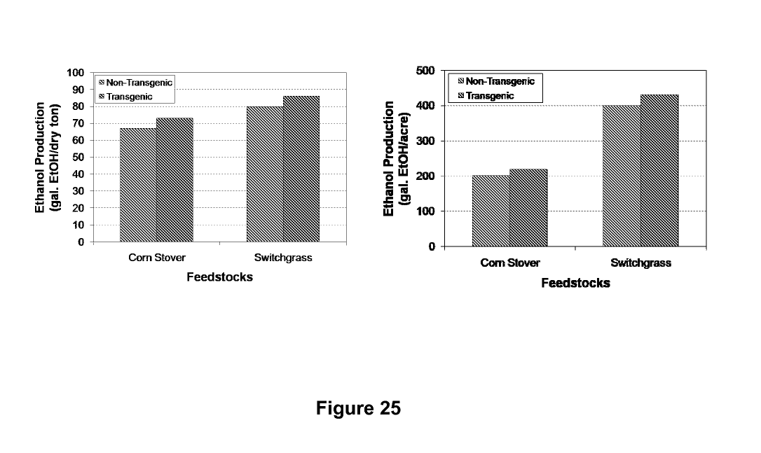

Bench scale experiments were performed to compare ethanol yields from transgenic E1 corn stover and E1 switchgrass against non-transgenic biomass. Various biomass samples were tempered, pretreated with dilute sulfuric acid, hydrolyzed with 0.2 mg/g of Accelerase 1500, and then fermented under identical conditions. A net 4% increase of ethanol yield was observed for the engineered E1 feedstocks compared to conventional feedstocks following 24 h of fermentation and extrapolated to a per ton yield (FIG. 25, left panel). This improved ethanol production suggests engineered switchgrass will be an especially promising biofuel feedstock based on the projected per acre biomass yields of the two feedstocks (three and seven tons per acre for corn and switchgrass respectively) (FIG. 25, right panel).

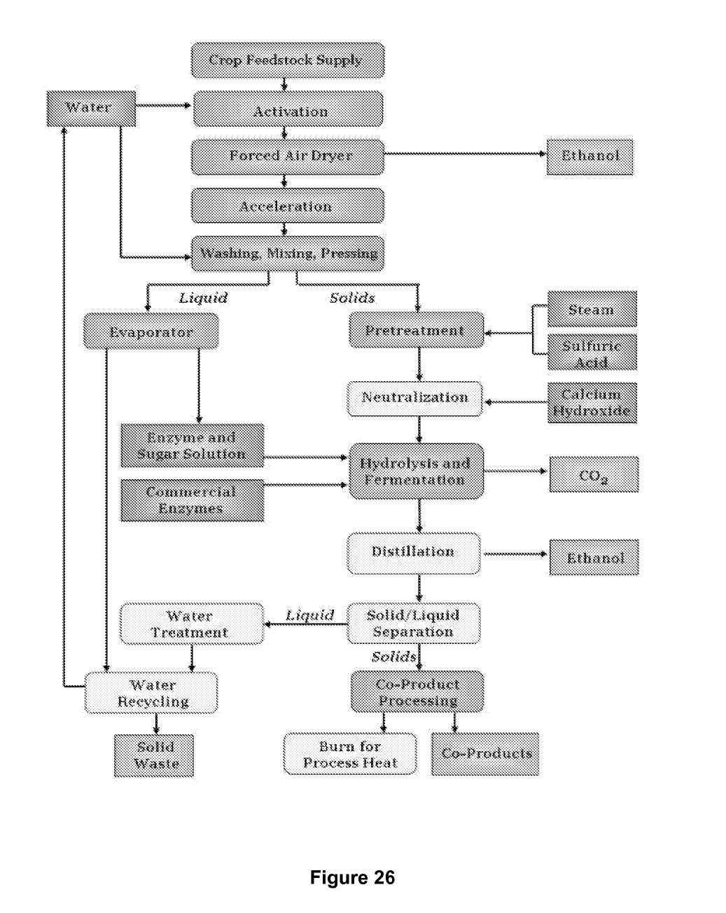

Results from experiments on conventional and transgenic biomass processed under standard and Edenspace unit process conditions indicate that a process like that shown in FIG. 26 could be used to increase glucan conversion, increase ethanol yield, reduce inhibitory compound formation, decrease severity of pretreatment, and lower the loading of commercial enzymes during saccharification.

### Example 15

### Alkaline Tempering Processes

The present Example illustrates that increased glucose release during enzyme hydrolysis can be achieved using a tempering process involving alkaline conditions.

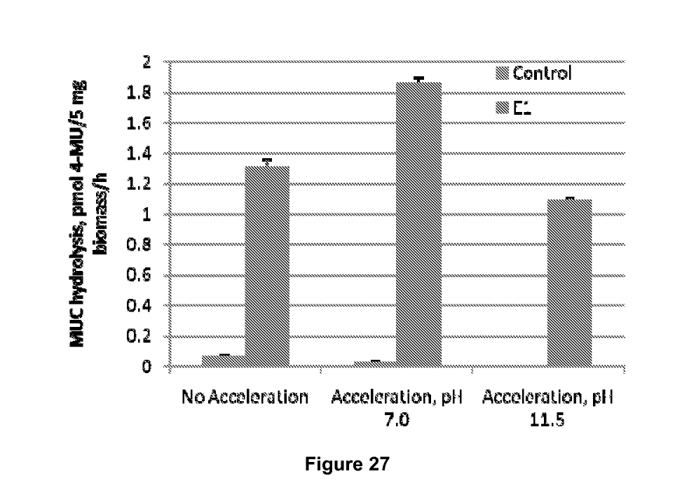

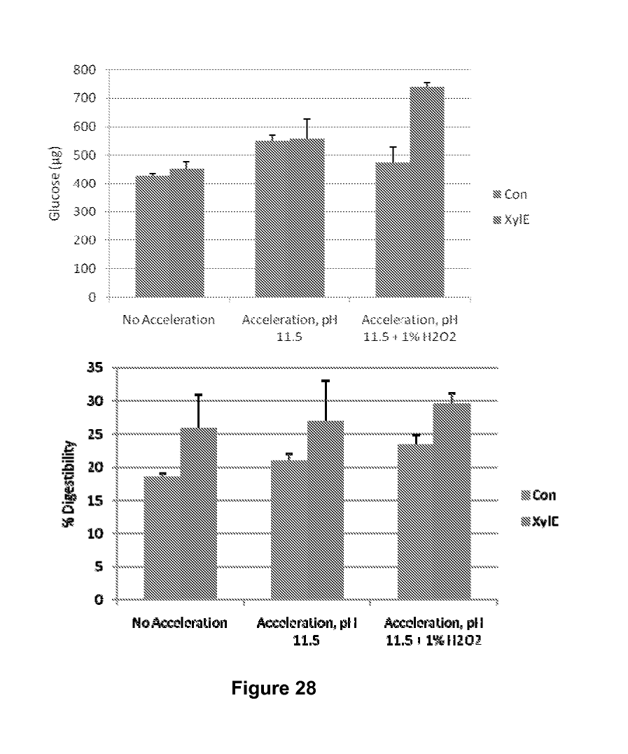

Previous reports have indicated that alkaline peroxide pretreatments are effective at removing lignin and increasing the enzyme conversion efficiency of corn stover as well as barley, wheat, and rice straw (Selig et al. (2009) “The effect of lignin removal by alkaline peroxide pretreatment on the susceptibility of corn stover to purified cellulolytic and xylanolytic enzymes.” Appl. Biochem. Biotechnol. 155:397-406); Sun et al. (2001) “Fractional and structural characterization of lignins in isolated by alkali and alkaline peroxide from barley straw.” J. Agric. Food Chem. 49:5322-30; Saha and Cotta (2006) “Ethanol production from alkaline peroxide pretreated enzymatically saccharified wheat straw.” Biotechnol. Prog. 22:449-53); and Sun et al. (2001) “Physiochemical characterization of lignins from rice straw by hydrogen peroxide treatment.” J. Appl. Polym. Sci. 79:719-32, the contents of each of which are herein incorporated by reference in their entirety). Our initial studies have demonstrated the compatibility of an alkaline peroxide delignification process with endoplant enzyme catalysis (FIGS. 27-28).

E1 biomass from trait positive (E1) and negative (Control) ED112.02D corn was harvested, dried, and milled to 1 mm particle size. Triplicate 5 mg samples of ground biomass material were weighed into microcentrifuge tubes for each experimental condition. Samples were either left dry or incubated at 65° C. in water (pH 7.0) or 50 mM phosphate buffer (pH 11.5) for 19 hours. Following incubation, samples were centrifuged, the supernatants were discarded, and the solids were rinsed with 0.5 mL 50 mM pH 5.0 to adjust the pH to pH 5.0 before measuring E1 endoglucanase activity in the solids. The dry, untreated samples were similarly rinsed with 0.5 mL 50 mM sodium acetate pH 5.0 just prior to determination of endoglucanase activity. Samples were incubated in the presence of 50 mM sodium acetate and 0.1 mM 4-methylumbelliferyl cellobioside (MUC) at 85° C. for 1 hour and then the residual E1 activity was measured through fluorescence.

As shown in FIG. 27, E1 endoglucanase survives tempering at both pH 7.0 and 11.5. Thus, E1 endoglucanses should be compatible with a process containing an alkaline delignification step followed by a readjustment of pH and temperature to those conditions that favor activity of E1 for hydrolysis.

To explore the advantages of tempering using another transgenically expressed enzyme polypeptide, biomass from corn stover expressing an endoxylanase from Acidothermus cellulolyticus (XylE) was then analyzed. Extractive compounds were removed from the transgenic and non-transgenic poplar biomass composite samples using a standard ethanol-acetone extraction procedure and dried to completeness in a fume hood. The weight of the extracted sample plus the tube was recorded. 50 mg of corn stover from biomass expressing XylE and control biomass lacking the XylE gene were incubated at 21° C. in 50 mM phosphate buffer (pH 11.5) in the absence or presence of 1% hydrogen peroxide for 24 hours. Following alkaline tempering, samples were neutralized, rinsed, and then subjected to enzymatic hydrolysis with 0.2 ml ACCELLERASE™ 1000/g.glucan) at 50° C. for 24 hrs. After 24 hours, glucose content in the supernatant was determined using a commercial glucose oxidase based assay kit.which and the solids rinsed extensively with water to remove hydrolyzed materials. Solids were dried to completeness and the final dry weight of the sample plus tube recorded. The amount of mass lost during the enzyme digestion was determined by subtracting the final sample weight from the starting weight. The % digestibility of a sample was defined as the amount of mass loss divided by the starting mass after ethanol/acetone extraction multiplied by 100.

As shown in FIG. 28, alkaline peroxide tempering of XylE corn biomass led to significant increases in glucose release during ACCELLERASE™ 1500 hydrolysis (top panel) and % digestibility (bottom panel).

### Example 16

### Tempering as an Enzyme Extraction Step

The present Example illustrates a process in which tempering is used as an enzyme extraction step.

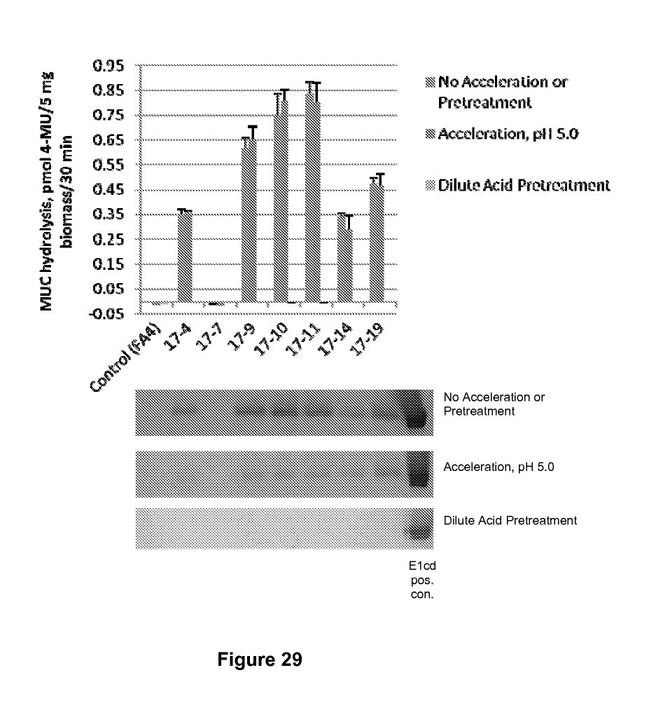

To identify critical limitations of transgenic plant feedstocks in current industrial processes, triplicate 5 mg samples of milled biomass from E1 and control switchgrass were subjected to one of the following treatments: no treatment (No Acceleration or Pretreatment control), incubation at pH 5.0 at 65° C. (Acceleration, pH5.0), or dilute acid pretreatment (1% sulfuric acid; 5% solids loading; 121° C.; 10 minutes). Following these process treatments, samples were centrifuged, the supernatant discarded, and the solids were rinsed with 0.5 mL 50 mM sodium acetate pH 5.0 before measuring the residual endoglucanase activity and E1 protein levels in the solids. To measure E1 activity, samples were incubated in the presence of 50 mM sodium acetate and 0.1 mM 4-methylumbelliferyl cellobioside (MUC) at 85° C. for 30 minutes prior to quenching with 1M sodium carbonate and measuring fluorescence due to 4-methylumbelliferone release. For E1 Western blots, rinsed residual solids were boiled in Laemmli buffer for five minutes and centrifuged, and the supernatant was loaded onto a 10% SDS-PAGE gel. Following electrophoresis, proteins in the gel were electrophoretically transferred to a PVDF membrane and subsequently immunoblotted with a monoclonal anti-E1 primary antibody and appropriate horseradish peroxidase (HRP)-labeled secondary antibody. Immunoreactive bands with visualized by an HRP-catalyzed reaction that converts a non-colored substrate into a purple colored precipitate in situ (FIG. 29, bottom panel).

Each of the processed solids from E1 switchgrass lines, except 17-7, had detectable levels of E1 activity and protein in the ‘No Acceleration or Pretreatment’ and ‘Acceleration, pH 5.0’ process treatment groups (FIG. 29, top panel). By contrast, no E1 activity or protein is associated with the solids following dilute acid pretreatment. This result indicates that tempering (e.g., acceleration) is compatible with the use of thermostable enzymes like E1, but that dilute acid pretreatment is too harsh for E1 survival.

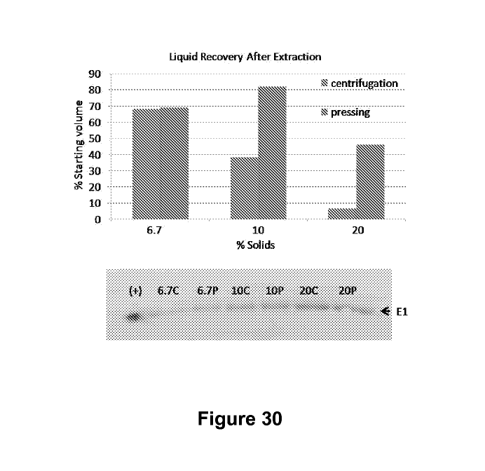

As a means to circumvent the damaging dilute acid pretreatment of solids, centrifugation and pressing of solids following acceleration at pH 5.0 and 70° C. for 24 hours were evaluated for the efficiency of E1 extraction. The liquid phase from centrifuged (C) or pressed (P) samples was analyzed by Western blot using a monoclonal anti-E1 primary antibody (FIG. 30, bottom panel). Pressing of solids, particularly at high solids loading levels, is a superior method to recover enzymes following tempering. These liquid phase enzymes can avoid damaging dilute acid pretreatment conditions experienced by solids and be recombined with solids after pretreatment along with commercial enzyme mixes to continue catalyzing biomass conversion. Re-addition of extracted enzymes will lead to reduced levels of commercial enzymes necessary for efficient hydrolysis of the pretreated biomass or for improved glucan conversion.

A known volume of E1 enzyme extracted from transgenic E1 poplar was subjected to the following treatments: no treatment, forced air evaporation in a standard food evaporator, vacuum concentration in a Speed-Vac rotary evaporator, or evaporation in a 70° C. dry heat block. The no treatment control (original extract) was stored at 4° C. until E1 activity was measured. Excepting the control, samples were concentrated to less than fifty percent of their starting volume, final volume was measured, volume of liquid used to measure E1 hydrolysis of 4-methylumbelliferyl-beta-D-cellobioside (MUC) was normalized for the degree of concentration, and activity in all samples was measured. For example, if a sample contained 100 μL of liquid before concentration and 50 μL after concentration, and if 10 μL of control, unconcentrated protein extract was used to measure the E1 activity of the original extract, then 5 μl of concentrated sample would be used to normalize volume of sample relative to the original volume. Release of 4-methylumbelliferone (4-MU) was measured using a fluorescent plate reader.

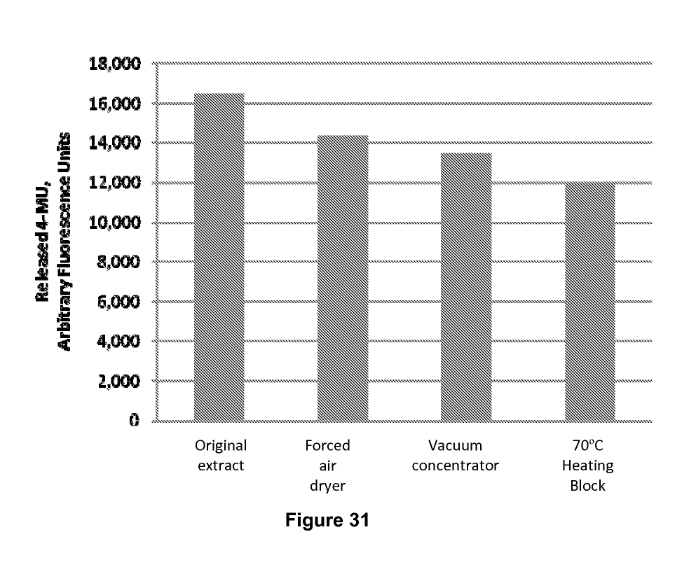

Activity measurements using normalized samples show that E1 activity retained at least 75% of its original activity against MUC after concentration by a variety of methods (FIG. 31).

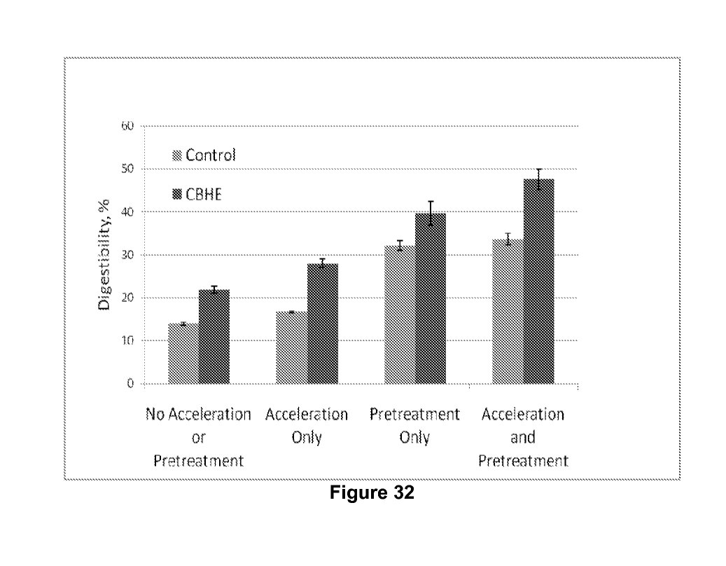

The acceleration process was also evaluated on poplar biomass expressing the CBHE exoglucanase. The tare weight of empty sample tubes was recorded and then milled transgenic and non-transgenic material (50 mg) was transferred to each tube and the dry weight of the sample plus the tube was recorded. Extractive compounds were removed from the transgenic and non-transgenic poplar biomass composite samples using a standard ethanol-acetone extraction procedure and dried to completeness in a fume hood. The weight of the extracted sample plus the tube was recorded. Samples were then treated according to their experimental group: ‘No Acceleration or Pretreatment’, ‘Acceleration Only’, ‘Pretreatment Only’, or ‘Acceleration and Pretreatment’ before enzymatic hydrolysis with Accelerase 1500. In FIG. 32, the exoglucanase (CBHE) and control samples in the ‘Acceleration Only’ and ‘Acceleration and Pretreatment’ groups were incubated for 24 h at 50° C., pH 5.0, following which the samples were rinsed and subjected to enzymatic hydrolysis with ACCELLERASE™ 1500 (Genencor) at a dose of 0.2 mL/g of glucan. Samples receiving pretreatment were reconstituted in 1% sulfuric acid and heated at 120° C. for 10 minutes followed by neutralization with 5 N sodium hydroxide. Following the indicated process treatments, all samples were incubated with 0.2 mL/g glucan Accelerase 1500 at 50° C. for 24 h after which time the solids rinsed extensively with water to remove hydrolyzed materials liberated during the 24 h hydrolysis period. Samples were dried to completeness and the final dry weight of the sample plus tube recorded. The amount of mass lost during the enzyme digestion was determined by subtracting the final sample weight from the starting weight. The digestibility of a sample was determined by calculating percentage of mass lost during the in vitro dry matter digestibility (IVDMD) procedure.

### Example 17

### Further Hydrolysis Studies on Tempered and Pretreated Transgenic Tobacco

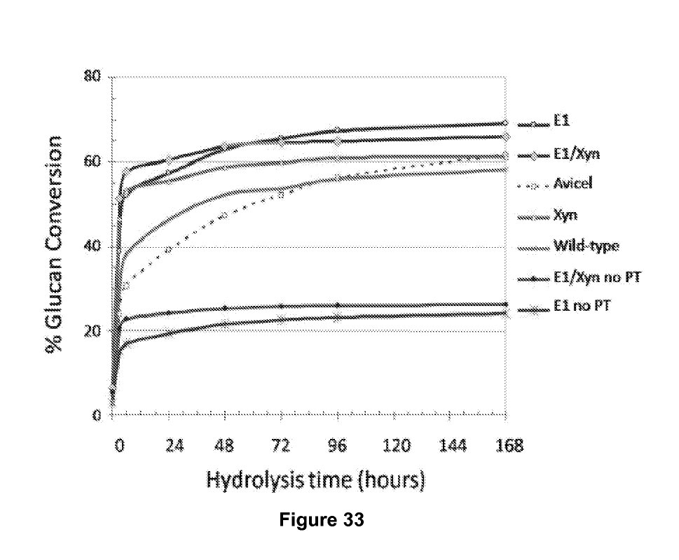

Example 7 showed that tempering and pretreatment of transgenic tobacco, particularly of E1+XynZ double transgenic tobacco, may lead to greater cellulose accessibility to commercial enzymes. In the present Example, followup hydrolysis studies were performed with ACCELLERASE™ 1000. Enzymatic hydrolysis of tempered but not pretreated and of tempered plus pretreated tobacco samples was conducted following the NREL LAP procedure (Selig et al., 2009). The cellulose load in this study was 2-3.5% (w/v) (7-10% dry mass load), the buffer solution was 20 mM pH 5.0 sodium acetate buffer with 0.05% NaN₃, and the enzyme was ACCELLERASE™ 1000 (0.5 mL/g of cellulose) from Genencor. Hydrolysis was conducted in sealed serum bottles in a 50° C. reciprocal water bath shaker running at 100 rpm for 168 hours. Samples were taken consecutively at designated time intervals, boiled in a boiling water bath for 5 min and centrifuged at 2,500 rpm for 5 min. The centrifuged supernatants were diluted by mixing 200 μL supernatant with 800 μL deionized water, filtered through 0.2 μm syringe filter, and analyzed for glucose, xylose, and arabinose using HPLC. Hydrolysis kinetics of the tested samples and Avicel (Sigma) are shown in FIG. 33.

Cellulose in the tobacco samples subjected to tempering plus pretreatment was highly reactive. Once enzyme was added into the hydrolysis buffer, 2-5% of the glucan was hydrolyzed by the time a zero hour sample could be collected (FIG. 33). Hydrolysis of glucan in sample (E1+XynZ) was the fastest. Three hours after addition of enzyme, 50% of glucan in that sample was hydrolyzed, and over 60% glucan was hydrolyzed around 24 hours. The hydrolysis rate for most samples leveled off after 48 hours, except for the rate for the Avicel sample, which still had glucose releasing even after 168 hours. This effect could be due to its low hemicellulose content and on lignin. Although E1+XynZ tobacco had the fastest hydrolysis rate in the beginning, E1 tobacco had the highest hydrolysis rate of 69.3% in the end. By the end of the hydrolysis test, the E1 and E1+XynZ samples had glucan hydrolysis yields of more than 65%, and the XynZ, wild-type control, and Avicel samples had hydrolysis yields around 60%. The two tempered but not acid pretreated samples, E1+XynZ and E1, had a glucan hydrolysis yields around 25%, indicating that pretreatment is still desirable following tempering of transgenic samples to improve glucan hydrolysis by cellulases. Compared with the tempered plus pretreated samples, glucan hydrolysis yields of around 25% were very low, but they were still much better than those of the non-tempered, non-pretreated samples (6.1% for non-transgenic control and 15.6% for E1 tobacco (data not shown)).

The rapid hydrolysis kinetics and observation that glucan conversion of transgenic samples reaches a plateau by 48 h suggests that relatively short saccharification duration could be employed in a commercial biorefining process using tempering and pretreatment.

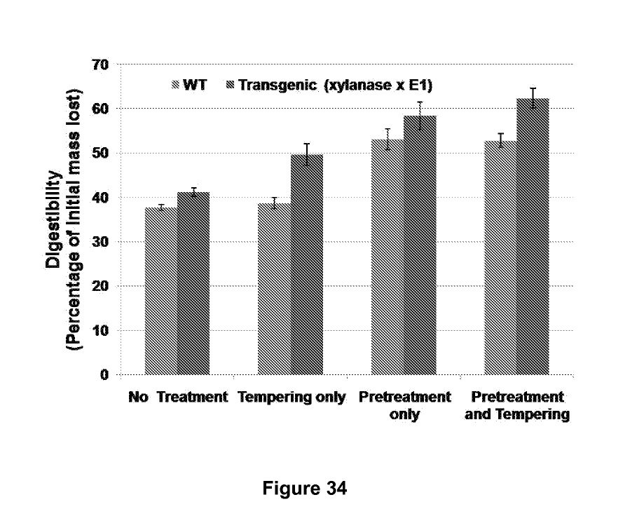

To obtain an initial indication of the compatibility of tempering with downstream processes, the digestibility of control and double transgenic E1+XynZ tobacco was compared when a variety of different pre-processing steps preceded enzyme hydrolysis with Celluclast and β-glucosidase. Digestibility of E1+XynZ tobacco significantly increased following tempering for 24 h at 70° C. before saccharification with Celluclast but the digestibility of control tobacco did not change with tempering treatment (FIG. 34). Dilute acid pretreatment (0.25% H₂SO₄, 121° C., 10 min) significantly increased the digestibility of both control and E1+XynZ tobacco compared to samples receiving no pre-processing treatment. The combination of tempering plus pretreatment showed further increases in the digestibility of E1+XynZ tobacco, indicating that tempering is a useful technology that has the potential to improve downstream processing steps for enzyme-producing crops.

### Example 18

### Tempering Processes Can be Used to Convert UInsoluble Fiber in Grain and Cobs from Plants Expressing Enzymes

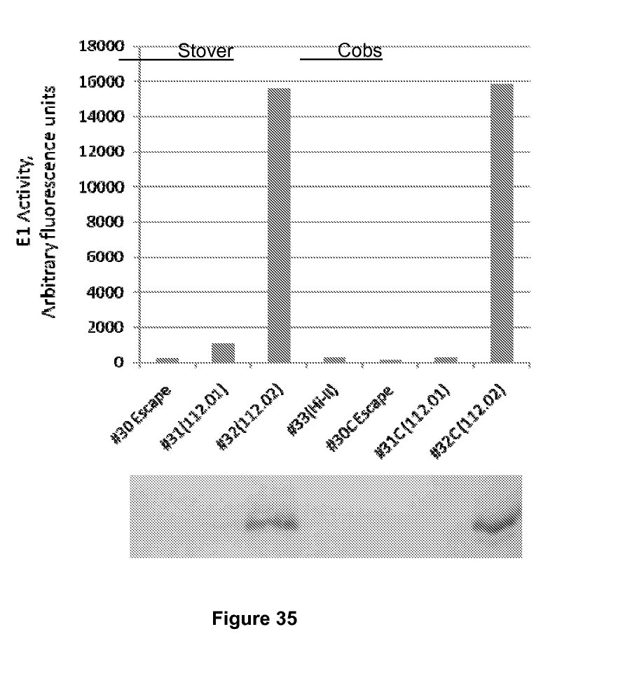

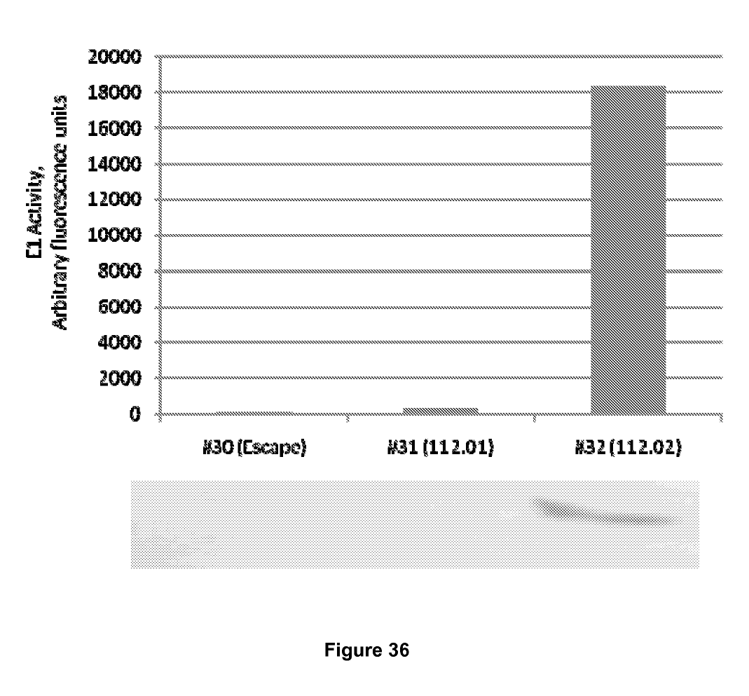

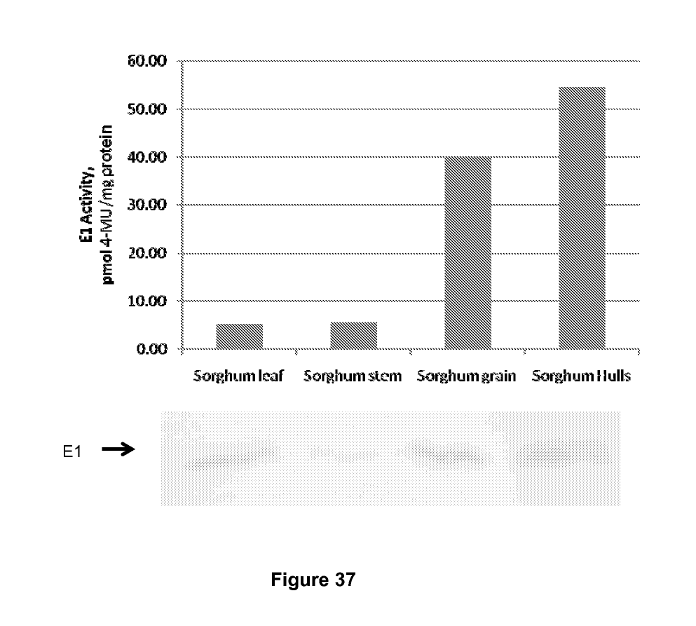

The fiber in corn grain and cobs is an attractive source of cellulose, as it is currently not utilized in existing corn grain biorefineries to produce ethanol. The presence of the E1 enzyme can be observed by Western blot, or by its activity on 4-methylumbelliferyl cellobioside (MUC). Significant amounts of E1 were observed in the cobs (FIG. 35) and milled grain (FIG. 36) from trait positive corn plants (ED112.02). Sorghum is another grain crop grown in the United States that has been commonly used for grain ethanol production. Sorghum was transformed with the E1 gene construct used to generate E1 corn line ED112.02, and a trait positive transgenic sorghum line (AB132.02F) identified. AB132.02F plants exhibited high levels of E1 in their grain and seed hulls (FIG. 37). Expression of E1 in these tissues will allow the application of tempering processes to convert insoluble fiber in existing grain biorefineries into free sugars.

The acceleration process was also evaluated on pericarps isolated from the grain of E1 transgenic plants as well as its relevant negative control. The tare weight of empty sample tubes was recorded and then milled transgenic and non-transgenic material (50 mg) was transferred to each tube and the dry weight of the sample plus the tube was recorded. Extractive compounds were removed from the transgenic and non-transgenic poplar biomass composite samples using a standard ethanol-acetone extraction procedure and dried to completeness. The weight of the extracted sample plus the tube was recorded. Samples were then subjected to acceleration (50° C., pH 5.0, for 24 hours) and dilute acid pretreatment (1% H₂SO₄, 120° C., 10 min) before enzymatic hydrolysis with 0.2 mL/g glucan Accelerase 1500. Samples were incubated in the presence of Accelerase 1500 at 50° C. for 24 h after which time solids were rinsed extensively with water to remove hydrolyzed materials liberated during the 24 h hydrolysis period. Samples were dried to completeness and the final dry weight of the sample plus tube recorded. The amount of mass lost during the enzyme digestion was determined by subtracting the final sample weight from the starting weight. Digestibility of a sample was determined by calculating percentage of mass lost during the in vitro dry matter digestibility procedure.

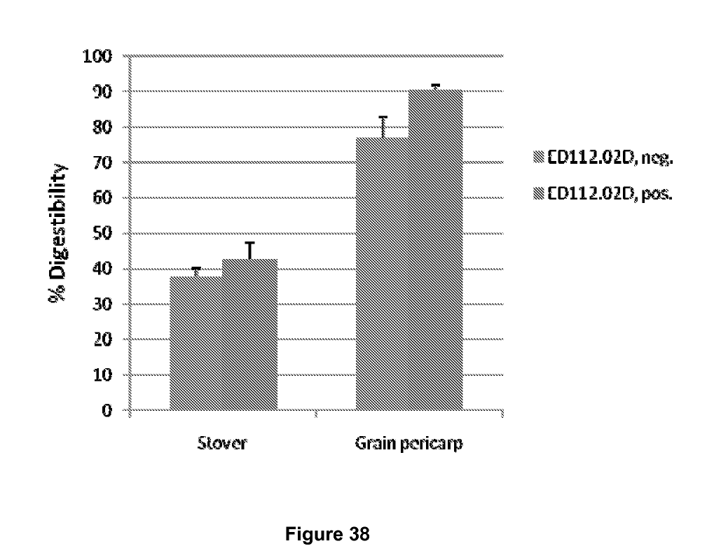

As seen in FIG. 38, following acceleration and pretreatment, E1 corn stover (ED112.02D, pos.) was slightly more digestible than negative control stover (ED112.02D, neg.), but this difference did not reach statistical significance. On the other hand, pericarp tissue isolated from E1 (ED112.02D, pos.) grain was significantly more digestible than control grain pericarp tissue (ED112.02D, neg.) (p<0.02, unpaired, two way t test). These data indicate that the acceleration and pretreatment process can be used to improve the digestion of fiber from the grain of transgenic plants expressing E1. Grain fiber is known to be rich in cellulose and hemicellulose while having very little lignin. The low lignin content of grain fiber makes it considerably less recalcitrant to enzymatic conversion. As demonstrated in FIGS. 31 and 35-38, active E1 enzyme is readily extracted from poplar, sorghum, especially sorghum grain and hulls, as well as corn stover, cobs, and grain. As indicated in FIG. 30, it is possible to increase the recovery of E1 in the liquid phase by pressing or centrifuging solids in a slurry. E1 enzyme activity remains stable after concentrating samples using a forced air dryer, vacuum concentrator, or by simply evaporating by mild heating (FIG. 31).

Together, these data indicate the feasibility of all steps constituting by-pass of enzymes in the liquid phase around the pretreatment of solids as well as their concentration before addition to neutralized pretreated solids for hydrolysis and fermentation, as shown in FIG. 26.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

What is claimed is:

1. A method for cost-effective processing of lignocellulosic biomass comprising steps of:
tempering a sample of plant biomass under conditions to promote activation of lignocellulolytic enzyme polypeptides present in the sample of plant biomass;
pretreating the sample under conditions to promote accessibility of celluloses within the lignocellulosic biomass; and
treating the pretreated sample under conditions that promote hydrolysis of cellulose to fermentable sugars,
wherein the sample of plant biomass is obtained from at least one transgenic plant, the genome of which is augmented with:
a recombinant polynucleotide encoding at least one lignocellulolytic enzyme polypeptide operably linked to a promoter sequence, wherein the polynucleotide is optimized for expression in the plant.

2. The method of claim 1, wherein the step of tempering comprises a process selected from the group consisting of ensilement, grinding, pelleting, microwaving, sonication, incubation at a specific temperature, incubation at a specific pH, and combinations thereof.

3. The method of claim 2, wherein the step of tempering comprises incubating the sample at a temperature between about 25° C. and about 100° C. for at least 1 hour.

4. The method of claim 3, wherein the step of tempering comprises incubating the sample at about 85° C. for at least 5 hours.

5. The method of claim 4, wherein the step of tempering comprises incubating the sample at about 85° C. for at least 24 hours.

6. The method of claim 2, wherein the step of tempering comprises ensiling the sample of plant biomass.

7. The method of claim 6, wherein the sample is ensiled for at least 5 days.

8. The method of claim 7, wherein the sample is ensiled for at least 10 days.

9. The method of claim 8, wherein the sample is ensiled for at least 20 days.

10. The method of claim 6, wherein the sample is ensiled at about 37° C.

11. The method of claim 6, wherein the sample is ensiled at about 85° C.

12. The method of claim 1, wherein the sample of plant biomass is in solid form during the step of tempering.

13. The method of claim 1, wherein the sample of plant biomass is in a liquid slurry during the step of tempering.

14. The method of claim 1, wherein the step of treating comprises externally applying an amount of at least one lignocellulolytic enzyme polypeptide.

15. The method of claim 14, wherein the amount of externally applied lignocellulolytic enzyme polypeptide required to achieve a given level of hydrolysis is less than the amount of externally applied lignocellulolytic enzyme polypeptide required to achieve the same level of hydrolysis of comparable lignocellulosic biomass from a plant part that is not obtained from a transgenic plant.

16. The method of claim 14, wherein the step of treating comprises externally applying an amount of at least two lignocellulolytic enzyme polypeptides, wherein the at least two lignocellulolytic enzyme polypeptides together have at least two different enzyme activities.

17. The method of claim 14, wherein the externally applied lignocellulolytic enzyme polypeptide has at least two different enzyme activities.

18. The method of claim 16 or 17, wherein the at least two different enzyme activities are selected from the group consisting of feruloyl esterase, xylanase, alpha-L-arabinofuranosidase, endogalactanase, acetylxylan esterase, beta-xylosidase, xyloglucanase, glucuronoyl esterase, endo-1,5-alpha-L-arabinosidase, pectin methylesterase, endopolygalacturonase, exopolygalacturonase, pectin lyase, pectate lyase, rhamnogalacturonan lyase, pectin acetylesterase, alpha-L-rhamnosidase, mannanase exoglucanase, cellulase, licheninase, laminarinase, beta-(1,3)-(1,4)-glucanase or beta-glucosidase, and combinations thereof.

19. The method of claim 18, wherein the at least two different enzyme activities comprise exoglucanase, endoglucanase, hemicellulase, and beta-glucosidase.

20. The method of claim 14, wherein the at least one externally applied lignocellulolytic enzyme polypeptide is capable of hydrolyzing cellulose to glucose monomers.

21. The methods of claims 1, wherein a greater level of hydrolysis is obtained from the sample of plant biomass obtained from transgenic plant than from a sample of plant biomass from a non-transgenic plant that is processed under the same conditions.

22. The method of claim 1, wherein the promoter sequence is a sequence of a promoter selected from the group consisting of a constitutive promoter, a developmentally-specific promoter, a tissue-specific promoter, and an inducible promoter.

23. The method of claim 22, wherein the promoter sequence is a sequence of a constitutive promoter selected from the group consisting of the act1 promoter and the 35S CMV promoter.

24. The method of claim 1, wherein the lignocellulolytic enzyme polypeptide is expressed in one or more targeted sub-cellular compartments or organelles.

25. The method of claim 24, wherein the one or more targeted sub-cellular compartments or organelles is selected from the group consisting of the apoplast, chloroplast, vacuole, cell wall, endoplasmic reticulum, and combinations thereof.

26. The method of claim 24, wherein the gene encoding the lignocellulolytic enzyme polypeptide is fused to a signal peptide sequence.

27. The method of claim 24, wherein the signal peptide sequence encodes a secretion signal that allows localization to a cell compartment or organelle selected from the group consisting of cytosol, vacuole, nucleus, endoplasmic reticulum, cell wall, mitochondria, apoplast, peroxisomes, and plastid.

28. The method of claim 27, wherein the signal peptide sequence encodes a secretion signal from sea anemone equistatin.

29. The method of claim 27, wherein the signal peptide sequence encodes a secretion signal comprising a KDEL motif

30. The method of claim 24, wherein the lignocellulolytic enzyme polypeptide is expressed in a plant part selected from the group consisting of stems, leaves, grain, cobs, and combinations thereof.

31. The method of claim 1, wherein the plant is selected from the group consisting of corn, switchgrass, sorghum, miscanthus, sugarcane, poplar, pine, wheat, rice, soy, cotton, barley, turf grass, tobacco, bamboo, rape, sugar beet, sunflower, willow, and eucalyptus.

32. The method of claim 31, wherein the plant is a corn plant.

33. The method of claim 31, wherein the plant is a switchgrass plant.

34. The method of claim 31, wherein the plant is a sorghum plant.

35. The method of claim 1, wherein the lignocellulolytic enzyme polypeptide is selected from the group consisting of cellulases, hemicellulases, ligninases, and combinations thereof.

36. The method of claim 1, wherein the lignocellulolytic enzyme polypeptide is selected from the group consisting of cellobiohydrolases, endoglucanases, β-D-glucosidases, β-D-glucan gluchydrolases, xylanases, xyloglucanses, β-xylosidases, arabinofuranosidases, acetyl xylan esterases, glucuronidases, rhamnogalacturonases, polygalacturonases, pectin methyl esterases, mannanases, galactanases, arabinases, lignin peroxidases, manganese-dependent peroxidases, hybrid peroxidases, laccases, ferulic acid esterases, glucuronyl esterases, isomerases, and combinations thereof.

37. The method of claim 36, wherein the lignocellulolytic enzyme polypeptide comprises an endoglucanase.

38. The method of claim 37, wherein the endoglucanase comprises E1 endo-1,4-β-glucanase.

39. The method of claim 38, wherein the amino acid sequence of the E1 endo-1,4-β-glucanase comprises the sequence of SEQ ID NO. 1.

40. The method of claim 36, wherein the lignocellulolytic enzyme polypeptide comprises a xylanase.

41. The method of claim 37, wherein the lignocellulolytic enzyme polypeptide comprises Xyn Z.

42. The method of claim 39, wherein the amino acid sequence of the Xyn Z comprises the sequence of SEQ ID NO. 5.

43. The method of claim 1, wherein the step of tempering comprises externally applying an amount of at least one lignocellulolytic enzyme polypeptide.

44. The method of claim 43, wherein the step of tempering comprises externally applying an amount of at least two lignocellulolytic enzyme polypeptides, wherein the at least two lignocellulolytic enzyme polypeptides together have at least two different enzyme activities.

45. The method of claim 1, wherein the step of treatment further comprises adding a crude extract from a sample of plant biomass, wherein the sample is obtained from at least one transgenic plant, the genomic of which is augmented with:
a recombinant polynucleotide encoding at least one lignocellulolytic enzyme polypeptide operably linked to a promoter sequence, wherein the polynucleotide is optimized for expression in the plant,
and wherein the crude extract comprises at least one lignocellulolytic enzyme polypeptide encoded by the recombinant polynucelotide.

46. The method of claim 45, wherein the crude extract comprises at least one lignocellulolytic enzyme fused to an affinity tag.

47. The method of claim 46, where in the affinity tag comprises a tag selected from the group consisting of HAT (histidine affinity tag), FLAG, c-myc, hemagglutinin antigen, and His.

48. The method of claim 1, wherein the sample of plant biomass comprises insoluble fiber.

49. The method of claim 1 or 43, wherein the sample of plant biomass is obtained from the grain of the transgenic plant.
